(19) United States
(12) Patent Application Publication
(10) Pub. No.: US 2003/0104443 A1
(43) Pub. Date: Jun. 5, 2003

(54) AFAP sequences, polypeptides, antibodies and methods

(75) Inventor: Daniel Flynn, Morgantown, WV (US)

Correspondence Address: PALMER & DODGE, LLP, PAULA CAMPBELL EVANS, 111 HUNTINGTON AVENUE, BOSTON, MA, 02199, US

(73) Assignee: University of West Virginia

(21) Appl. No.: 10/246,354

(22) Filed: Sep. 18, 2002

Related U.S. Application Data
(60) Provisional application No. 60/323,866, filed on Sep. 21, 2001.

(51) Int. Cl. C12Q 1/68; G01N 33/574; C07H 21/04; C12P 21/02; C12N 5/06; C12N 9/20

(52) U.S. Cl. 435/6; 435/7.23; 435/69.1; 435/198; 435/320.1; 435/325; 530/388.26; 536/23.2

(57) ABSTRACT

The present invention comprises reagents and methods which target actin filaments or the cellular signals that control actin filament integrity. Specifically, the invention provides novel actin binding polypeptides (e.g., human AFAP polypeptides), antibodies which specifically recognize the same, nucleic acids encoding the same, and methods for using these polypeptides, antibodies, and nucleic acids. Such reagents are useful for the diagnosis, prognosis and treatment of cancer, and in the research and development of anti-cancer diagnostic and therapeutic agents. Such reagents are useful for treatment of conditions associated with aberrant angiogenesis, for example, obesity. In one aspect, model animals are provided comprising AFAP regulatory sequences operably linked to reporter molecules. These animals provide a system in which to study the affect of agents on neural development.

### PRIORITY

This application claims priority to 60/323,866, filed on Sep. 21, 2001.

### FIELD OF THE INVENTION

The invention relates to novel actin binding polypeptides (AFAP polypeptides), antibodies which specifically recognize the same, nucleic acids encoding the same, and uses for these polypeptides, antibodies, and nucleic acids.

### BACKGROUND OF THE INVENTION

The cSrc (“Src”) nonreceptor tyrosine kinase is normally repressed and inactive in cells; however, during the G2/M transition, or responsive to growth factor receptor stimulation, Src becomes activated, concomitant with a relaxation of actin filament structures. Src is activated in several human cancer cell lines (Bolen et al., 1987, Proc. Natl. Acad. Sci. USA 84: 2251-2255; Boschek et al., 1981, Cell 24: 175-184; Cartwright et al., 1990, Proc. Natl. Acad. Sci. USA 87: 558-562; Irby et al. 1999, Nat. Genet. 21: 187-190; Rosen et al., 1986, J Biol. Chem. 261: 13754-13759; Tarone et al., 1985, Exp. Cell. Res. 159: 141-157) and one of the hallmarks of transformation by activated forms of Src is the dissolution of stress filaments and a repositioning of actin into rosette-like structures (Reynolds et al., 1989, Mol. Cell. Biol. 9: 3951-3958; Felice et al., 1990, Eur. J Cell Biol. 52: 47-59). Antisense vectors that reduce Src expression in the HT29 human colon cancer cell lines will significantly reduce the transformed properties of these lines and drugs that block Src will impede progression through the G2/M transition. These data demonstrate a role for Src in modulating signals that affect cell growth and motility.

The cSrc proto-oncogene can be activated by dephosphorylation of Tyr⁵²⁷ by cellular phosphatases, or displacement of repressive, intramolecular interactions involving the SH2 and SH3 domains (Brown and Cooper, 1996, Biochim. Biophys. Acta, 1287:121-149). These activation events normally occur in response to cellular signals, e.g., growth factors interacting with their receptors (Brown and Cooper, 1996, supra). These pathways are thought to proceed through Src, with the subsequent phosphorylation of substrates and activation of downstream signaling members, including Ras (He et al., 2000), ppl25^{FAK} (Thomas et al., 1998, Exp. Cell Res., 159:141-157), Crk (Sabe et al., 1992, Mol. Cell Biol., 12: 4706-4713) and ppl30^{Cas} (Xing et al., 2000, Mol. Cell Biol., 20: 7363-7377).

Downstream signaling proteins can modulate the effects of activated Src. For example, Src can be activated by dephosphorylation of Tyr⁵²⁷ by cellular phosphatases, or displacement of repressive, intramolecular interactions involving the SH2 and SH3 domains (Brown and Cooper, 1996, Biochem. Biophys. Acta 1287: 121-149). These activation events usually occur in response to cellular signals, e.g., such as occurs when growth factors interact with their receptors (Brown and Cooper, supra). Activated Src regulates actin filament integrity via signal transduction pathways modulated by downstream effector proteins, including PKCα, PI 3-kinase, Ras (He et al., 2000, Cancer J. 6: 243-248), pp125^{FAK} (Thomas et al., 1998, J. Biol. Chem. 273: 577-583) Crk (Sabe et al., 1992, supra), Rho and pp130^{Cas} (Xing et al., 2000, supra). Activated forms of PKCα, PI 3-kinase, and Ras can initiate changes in actin filaments similar to the effects of Src^{527F}. In addition, activation of Src will direct a down-regulation of Rho activity. While dominant negative forms of PKCα, PI 3-kinase, and Ras, will block the effects of Src^{527F} upon actin filaments, dominant-positive forms of Rho will direct the formation of well-formed stress fibers and block the ability of Src^{527F} to alter actin filament integrity.

The actin filament associated protein AFAP-110 is a tyrosine phosphorylated substrate of Src and is an SH2/SH3 binding partner for Src^{527F} (Flynn et al., 1993, Mol. Cell. Biol. 13: 7982-7900). AFAP-110 is an adaptor protein that binds to actin filaments via a carboxy terminal, actin binding domain and colocalizes with stress filaments and the cortical actin matrix along the cell membrane (Quin et al., 1998, Oncogene, 16: 2185-2195; Quin et al., 2000, Exp. Cell. Res., 255:1-2-113). AFAP-110 also is capable of being an SH2/SH3 binding partner for cFyn and cLyn (Flynn et al., 1993, supra; Guappone and Flynn, 1997, Mol. Carinogen. 22: 110-119). In addition to SH2 and SH3 binding motifs, AFAP comprises two pleckstrin homology domains (PH1 and PH2), a carboxy terminal leucine zipper, which facilitates self association of AFAP-110 (Quin et al., 1998, supra) and an actin binding domain (Flynn et al., supra, Qian et al, 2000, supra). AFAP-110 also contains a target region for serine/threonine phosphorylation as well as other hypothetical protein-binding sites (Baisden et al., 2001a, Oncogene, 20:6435-6447). AFAP-110 is hyperphosphorylated on ser/thr residues as well as tyrosine residues in Src transformed cells and contains numerous consensus sequences for phosphorylation by PKC (Kanner et al., 1991, EMBO J., 10:1689-1698; Flynn et al., 1993, supra). AFAP-110 appears to function as an adapter molecule linking a variety of signaling proteins to the actin cytoskeleton.

The carboxy terminal leucine zipper motif appears to play a regulatory role for AFAP-110. In Src^{527F} transformed cells, AFAP-110 undergoes a conformational change that affects its ability to self-associate via the leucine zipper motif (Qian et al., 1998 supra). This conformational change was detected using two separate assays that demonstrate changes in self-association, affinity chromatography (affinity absorptions) and gel filtration analysis. Affinity absorption of AFAP-110 using the GST-cterm fusion proteins that encoded the leucine zipper motif, will bind cellular AFAP-110. Deletion of the leucine zipper motif from GST-cterm prevents affinity absorption. In Src^{527F} transformed cell, GST-cterm can no longer bind cellular AFAP-110, indicating that Src^{527F} directed cellular signals which affected a change in AFAP-110 conformation, precluding access to an intramolecular binding site for the leucine zipper motif. Gel filtration analysis confirmed that cellular AFAP-110 does self-associate, existing predominantly as a multimer (trimers, tetramers and possibly larger complexes), with a minor population predicted to be monomers. In Src^{527F}-transformed cells, AFAP-110 fractionated as a single population, predicted to represent dimers. Thus, Src 527F directed a cellular signal that alters AFAP-110 conformation. The cellular signal that affected this change in conformation of AFAP-110 appeared to occur independently of tyrosine phosphorylation. Co-expression of cSrc had no effect on the ability of GST-cterm to affinity absorb AFAP-110, even though cSrc was able to direct tyrosine phosphorylation of AFAP-110 (Qian et al., 1998 supra).

The functional significance of the leucine zipper motif was revealed by deletional mutagenesis and expression of AFAP-110^{Δlzip} and expression of AFAP-110^{Δlzip} in fibroblast cell lines. Here, AFAP-110^{Δlzip} induced a phenotype similar to Src^{527F}, resulting in a repositioning of actin filaments into rosette-like structures and the formation of motility structures (Qian et al., 1998 supra; Qian et al., 2000 supra). Subsequent data demonstrated that the mechanism by which AFAP-110^{Δlzip} was able to alter actin filament integrity was via its ability to activate cellular tyrosine kinases. However, engineering of a point mutation into AFAP-110^{Δlzip} that abrogated SH3 binding to Src (Pro⁷¹→Ala⁷¹; Guappone and Flynn, 1997 supra), prevented AFAP-110^{Δlzip/71A} from altering actin filament integrity. Previously, it had been shown that Src family kinases can be activated by engagement with SH3 binding partners. Activation is achieved by interfering with and relieving the repressive intramolecular interactions involving the SH3 domain of cSrc. The HIV Nef protein and Herpesvirus Tip protein have been shown to activate Src family members in an SH3-dependent fashion (Collette et al., 2000; Hartley et al., 1999; Moarefi et al., 1997). Both of these proteins transform cells, and the Tip protein is a requirement for the induction of tumors by the Herpesvirus (Duboise et al., 1998). The ability of AFAP-110^{Δlzip} to alter actin filament integrity was due to the subsequent activation of Rho, downstream of activated cSrc. Dominant positive RhoA^{V14} was able to impede the ability of AFAP-110^{Δlzip} from altering actin filament integrity, while being unable to effect induction of cSrc family activation or cellular tyrosine phosphorylation. These data indicated that AFAP-110^{Δlzip} was directing changes in actin filament integrity via SH3 mediated activation of cSrc with subsequent stimulation to the Rho family, which are well known effectors of actin filament integrity (Ridley 1992, Prog. Mol. Subcell. Biol., 22:1-22). Thus, it was hypothesized that AFAP-110 is a cSrc activating protein and the integrity of the leucine zipper motif was important for regulating this function. Changes in conformation could present AFAP-110 as an efficient SH3 binding partner for cSrc, enabling SH3-mediated activation of cSrc and subsequent changes in actin filament integrity.

An alternatively processed variant form of AFAP-110 was identified in brain, called AFAP-120, which contains an additional 86 amino acids of coding sequence placed near the carboxy terminus (Flynn et al., 1995, J. Biol. Chem., 270:3894-3899). The additional sequence, referred to as novel insert, neural specific (NINS), did not disrupt the reading frame of downstream carboxy terminal coding sequence common to both AFAP-110 and AFAP-120. The NINS was hypothesized to represent an additional protein binding module, based on its ability to affinity absorb a 67 kDa protein via an internal proline-rich region.

### SUMMARY OF THE INVENTION

The present invention comprises reagents and methods which target actin filaments or the cellular signals that control actin filament integrity. Specifically, the invention provides novel actin binding polypeptides (e.g., AFAP polypeptides), antibodies which specifically recognize the same, nucleic acids encoding the same, and methods for using these polypeptides, antibodies, and nucleic acids. Such reagents are useful for the diagnosis, prognosis and treatment of cancer, or obesity, and in the research and development of anti-cancer diagnostic and therapeutic agents and/or anti-obesity therapeutic agents. Such reagents are also useful for modulating angiogenesis. In one aspect, model animals are provided comprising AFAP regulatory sequences operably linked to reporter molecules. These animals provide a system in which to study the affect of agents on neural development.

In one aspect, the invention provides a nucleic acid sequence according to FIGS. 1A-C, FIG. 3C, GenBank Accession Nos. L20303 or L20302, or an RNA transcript corresponding to the nucleic acid sequence shown in FIGS. 1A-C, FIG. 3C, GenBank Accession Nos. L20303 or L20302. In another aspect a nucleic acid is provided which encodes an amino acid sequence according to FIG. 2, FIG. 3A, FIG. 3B, or a fragment thereof comprising at least one AFAP domain. The at least one AFAP domain preferably is selected from the group consisting of an AFAP-110 WW binding domain, an AFAP-110 SH2 binding domain; an AFAP-110 SH3 binding domain, an AFAP-110 PH1 and/or PH2 domain, an AFAP-110 leucine zipper domain, an AFAP-110 actin-binding domain, a serine/threonine kinase target domain, and a nuclear export domain. Preferably, at least one domain is from a human or mouse AFAP polypeptide In one aspect, the nucleic acid encodes an amino acid sequence according to FIG. 2 or FIGS. 3A and 3B comprising one or more insertions, deletions, and/or substitutions.

Complements of any of the foregoing nucleic acid sequences also are provided. In still another aspect, a sequence is provided which specifically hybridizes to any of the foregoing sequences under stringent conditions. The invention further provides nucleic acid sequences which are at least about 80%, 85%, 90% or 95% identical to the foregoing sequences and that have similar biological activity. In a preferred aspect, the nucleic acid sequences are greater than about 80% identical, and more preferably greater than about 87% identical, to a nucleic acid encoding a chicken AFAP-110 nucleic acid.

The invention also provides nucleic acid splice variants of a sequence according to FIGS. 1A-C or FIG. 3C, wherein the sequence comprises a sequence encoding a NINS amino acid sequence. In a preferred aspect, the NINS sequence is a human sequence. In one aspect, a nucleic acid molecule is provided which specifically hybridizes to an AFAP-120 transcript and not to an AFAP-110 transcript under stringent conditions.

The invention further provides any of the foregoing nucleic acids fused in-frame to a nucleic acid encoding a heterologous polypeptide, such as, for example, Green Fluorescent Protein (GFP).

A vector also is provided comprising any of the nucleic acids discussed above. Preferably, the vector is an expression vector.

The invention also provides a host cell comprising any of the nucleic acids described above, a tissue comprising the host cell, and a non-human animal comprising the host cell.

The invention further provides a method for producing a polypeptide comprising introducing the expression vector into a host cell and culturing the host cell under conditions which allow for the expression of the polypeptide. The invention also provides polypeptides produced by the method. In one aspect, the polypeptide is phosphorylated.

The invention further provides an antibody, including an antibody fragment or single chain antibody, which specifically recognizes the polypeptide as well as hybridoma cells producing the antibody. In a preferred aspect, the antibody recognizes a phosphorylated form of the polypeptide but does not recognize a non-phosphorylated form of the polypeptide. In a particularly preferred aspect, the antibody modulates one or more AFAP activities upon binding to an AFAP polypeptide.

In another aspect, the antibody specifically recognizes AFAP-120 and not AFAP-110. In still another aspect, the antibody recognizes an epitope within amino acids 1-55 of the human AFAP-110 polypeptide. In a further aspect, the antibody recognizes an epitope within an AFAP SH3 binding domain; a WW binding domain; an AFAP-110 SH2 binding domain; an AFAP-110 PH1 and/or PH2 domain; an AFAP-110 leucine zipper domain; an AFAP-110 actin-binding domain; an AFAP-110 serine/threonine kinase target domain; an AFAP-110 nuclear export domain; a modified form thereof; or a variant form thereof.

The invention also provides a method of screening for enhanced risk for a pathology associated with an aberrant AFAP signaling pathway in a test patient. The method comprises obtaining a biological sample from the patient, contacting the sample with a molecular probe reactive with an AFAP biomolecule, and detecting the reactivity of the molecular probe with the sample. The AFAP biomolecule can be a nucleic acid encoding an AFAP polypeptide or can be an AFAP polypeptide, modified form thereof, or variant form thereof. The molecular probe can be a nucleic acid or an antibody.

In one aspect, the molecular probe is used to quantitate the amount of an AFAP biomolecule. A decrease in the amount of an AFAP biomolecule in the sample from the test patient compared to the amount in a sample from a normal patient provides an indication of an increased risk for the presence of the pathology.

In another aspect, the molecular probe reacts with a modified form of the AFAP polypeptide but does not react with the unmodified form of the AFAP polypeptide and detection of the modified form indicates an increased risk for the presence of the pathology.

In still another aspect, the molecular probe is used to detect a mutant form of an AFAP gene, AFAP transcript, or AFAP polypeptide, and detection of the mutant form provides an indication of an increased risk for the pathology.

In a preferred aspect, the pathology is cancer, e.g., such as breast cancer, colon cancer, prostate cancer, lung cancer, a cancer involving neural cells, Ewing sarcoma and rhabdomyosarcoma.

In another aspect, the pathology is a neuropsychiatric disorder.

In a further aspect, the pathology is a disorder associated with the abnormal proliferation, differentiation, and/or death of hematopoietic cells or cells belonging to a hematopoietic cell lineage.

In a further aspect, the pathology is obesity.

The invention also provides a method for identifying an agent which is capable of binding to an AFAP polypeptide, modified form thereof, or variant thereof. The method comprises reacting an AFAP polypeptide, a fragment thereof comprising one or more AFAP domains, a modified form thereof, or a variant form thereof with at least one agent and detecting changes in one or more AFAP activities. In one aspect, the agent is an antibody or antigen-binding fragment thereof.

The invention further provides a method for identifying an agonist or antagonist of the interaction between an AFAP polypeptide, a fragment thereof comprising one or more AFAP domains, a modified form thereof, or variant thereof, and a substance which binds to the polypeptide, fragment, modified form thereof, or variant thereof. The method comprises providing a known concentration of the AFAP polypeptide, fragment thereof, modified form thereof, or variant thereof; providing a substance which is capable of specifically binding to the AFAP polypeptide, fragment thereof, modified form thereof, or variant thereof, under conditions which permit the formation of complexes between the substance and the AFAP polypeptide, fragment thereof, modified form thereof, or variant thereof, and assaying for one or more of: complexes, free substance, and non-complexed AFAP polypeptide, fragment thereof, modified form thereof, or variant thereof. Alternatively, or additionally, changes in one or more activities of an AFAP polypeptide, fragment thereof, modified form thereof, or variant form thereof can be monitored.

The substance can be a polypeptide or a second messenger molecule. In one aspect, the substance is Src, actin, a PKC polypeptide (e.g., such as PKCα, PKCβ, PKCγ, PKCλ), or Rho.

The method can be used to screen for an agonist or antagonist which constitutively activates Src. In a preferred aspect, an agonist or antagonist is identified which modulates the interactions of an AFAP polypeptide, fragment thereof, modified form thereof, or variant form thereof, with the SH3 domain of Src.

In another preferred aspect, the agonist or antagonist affects binding of an AFAP polypeptide comprising one or more PH domains, modified form thereof, or variant thereof, to a PKC polypeptide. Preferably, the PH domain is the PH1 domain.

The invention also provides a pharmaceutical composition comprising an agent, agonist or antagonist as described above. The pharmaceutical composition preferably is used to treat a patient having a pathology related to an aberrant AFAP signaling pathway, such as cancer or obesity, or a disorder resulting from increased or decreased angiogenesis.

The invention further provides transgenic animals comprising one or more AFAP regulatory sequences operatively linked to a reporter molecule. Preferably, the regulatory sequence (e.g., promoter, enhancer elements) is capable of driving the expression of the reporter molecule in neural cells in which AFAP polypeptides are normally expressed. In one aspect, the affect of an agent administered to the animal on the expression of the reporter molecule is monitored as a means of evaluating the effect of the agent on neuronal development.

### BRIEF DESCRIPTION OF THE DRAWINGS

The objects and features of the invention can be better understood with reference to the following detailed description and accompanying drawings.

FIGS. 1A-C show the coding sequence of human AFAP-110 from 5′ to 3′. Start and stop codons, respectively, are underlined. FIG. 1D shows the intron-exon structure of human AFAP-110.

FIG. 2 shows the amino acid sequence of human AFAP-110. The positions of intron-exon boundaries are indicated.

FIG. 3A shows the amino acid sequence of chicken AFAP 120 amino acids. FIG. 3B shows the amino acid sequence of the human NINS sequence in comparison with the chicken AFAP-120 amino acid sequence. FIG. 3C shows a nucleic acid sequence encoding chicken AFAP-120.

FIG. 4 demonstrates that expression of active PKC results in tyrosine hyperphosphorylation of Src.

FIG. 5 demonstrates that the activation of tyrosine hyperphosphorylation by PKC requires the integrity of both the SH3 binding motif and PH domain of AFAP-110.

FIG. 6 demonstrates that the activation of Src family kinases by PKC requires the integrity of both the SH3 binding motif and PH domain of AFAP-110.

FIG. 7 demonstrates that rAFAP-110 cooperatively binds to actin filaments.

FIG. 8 demonstrates that rAFAP-110 cross-links actin filaments through the carboxy terminal region.

FIG. 9 demonstrates that rAFAP-110 cross links actin filaments.

FIG. 10 demonstrates that AFAP-110 is a substrate of PKCα.

FIG. 11 demonstrates the AFAP-110 is a binding partner of PKCα.

FIG. 12 demonstrates that either PKC phosphorylation or leucine zipper deletion destabilizes AFAP-110 multimerization.

FIG. 13 demonstrates that either PKC phosphorylation or leucine zipper deletion increases AFAP-110's ability to cross-link actin filaments.

FIG. 14 demonstrates that AFAP-110 mediates the effects of PKC on actin filaments.

FIG. 15 demonstrates that AFAP-110^{Δlzip} expression results in the disruption of actin filaments and formation of rosettes.

FIG. 16 demonstrates that AFAP-110^{Δlzip} is hyperphosphorylated upon expression in Cos-1 cells.

FIG. 17 demonstrates that AFAP-110^{Δlzip} activates cellular tyrosine phosphorylation.

FIG. 18 demonstrates that AFAP-110^{Δlzip} activates Src family kinases.

FIG. 19 demonstrates that abrogation of SH3 binding inhibits the cytoskeletal rearrangement and activation of Src family kinases by AFAP-110^{Δlzip}.

FIG. 20 demonstrates that deletion of part of the amino-terminal PH domain of AFAP-110 inhibits the cytoskeletal rearrangement and activation of Src family kinases by AFAP-110^{Δlzip}

FIG. 21 demonstrates that RhoA^{V14} overcomes the ability of AFAP-110^{Δlzip} to alter actin filaments.

FIG. 22 demonstrates that AFAP-110 activates cellular tyrosine phosphorylation and Src family kinase activation in the presence of RhoA^{V14}.

FIG. 23 is a model of the pathway of activation of cellular kinases by AFAP-110 in response to the conformational change induced by cellular signals or deletion of the leucine zipper.

FIG. 24 demonstrates that AFAP-110 is found in both the Triton-soluble and insoluble fractions.

FIG. 25 demonstrates that AFAP-110 is expressed in breast myoepithelial cells.

FIG. 26 demonstrates immunohistochemical localization of AFAP in E-16 mouse embryo head.

FIG. 27 demonstrates high magnification analysis of AFAP immunolocalization in E-16 mouse embryo head.

FIG. 28 demonstrates immunohistochemical localization of AFAP in P-3 mouse brain.

FIG. 29 demonstrates AFAP localization in P-3 mouse pups.

FIG. 30 demonstrates immunohistochemical localization of AFAP in adult mouse brain.

FIG. 31 demonstrates that AFAP-120 is a splice variant of AFAP-110.

FIG. 32 demonstrates the analysis and characterization of an antibody specific for AFAP-120.

### DETAILED DESCRIPTION

Novel AFAP polypeptides, antibodies which specifically recognize the same, nucleic acids encoding the same, and methods for using these polypeptides, antibodies, and nucleic acids are provided. Such reagents are useful for the diagnosis, prognosis and treatment of pathologies involving disrupted AFAP signaling pathways. Model animals also are provided which comprise AFAP regulatory sequences operably linked to reporter molecules. Such animals provide systems in which to study the effects of agents on neuronal development.

Definitions

The following definitions are provided for specific terms which are used in the following written description and claims.

As used herein, an “AFAP domain” refers to any of: an AFAP-110 WW binding domain; an AFAP-110 SH2 binding domain (at amino acids corresponding to amino acids 93-98 or 451-458 of the human sequence); an AFAP-110 SH3 binding domain (at amino acids corresponding to amino acids 59-74 of the human sequence); an AFAP-110 PH1 domain (at amino acids corresponding to amino acids 157-248 of the human sequence), an AFAP-110 PH2 domain (at amino acids corresponding to amino acids 347-450 of the human sequence); an AFAP-110 leucine zipper (at amino acids corresponding to 563-598 of the human sequence); an AFAP-110 actin-binding domain (at amino acids corresponding to amino acids 600-620 of the human sequence), an AFAP-110 serine/threonine kinase target domain (at amino acids corresponding to amino acids 250-400 of the human sequence), and an AFAP-110 nuclear export domain (at amino acids corresponding to amino acids 4-25 of the human sequence).

As used herein, “amino acids corresponding to” amino acids of the human sequence, refers to amino acids sequences that share a high degree of conservation to the domains of the human sequence (at least about 60%) and share substantially the same biological activity (e.g., an actin binding domain of a non-human sequence should be able to bind actin).

As used herein, “sequence identity” can be determined by commercially available computer programs that can calculate percent (%) identity between two or more sequences using any suitable algorithm for determining identity. Preferably, percent identity is determined after maximally aligning sequences using methods and programs routine in the art.

As used herein, a “heterologous polypeptide” refers to an amino acid sequence not normally found adjacent to a native polypeptide sequence in a cell.

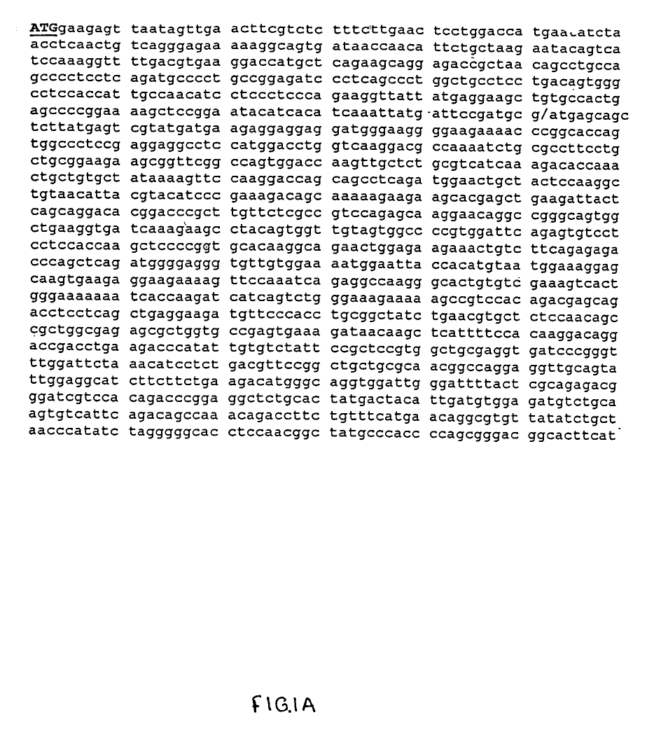

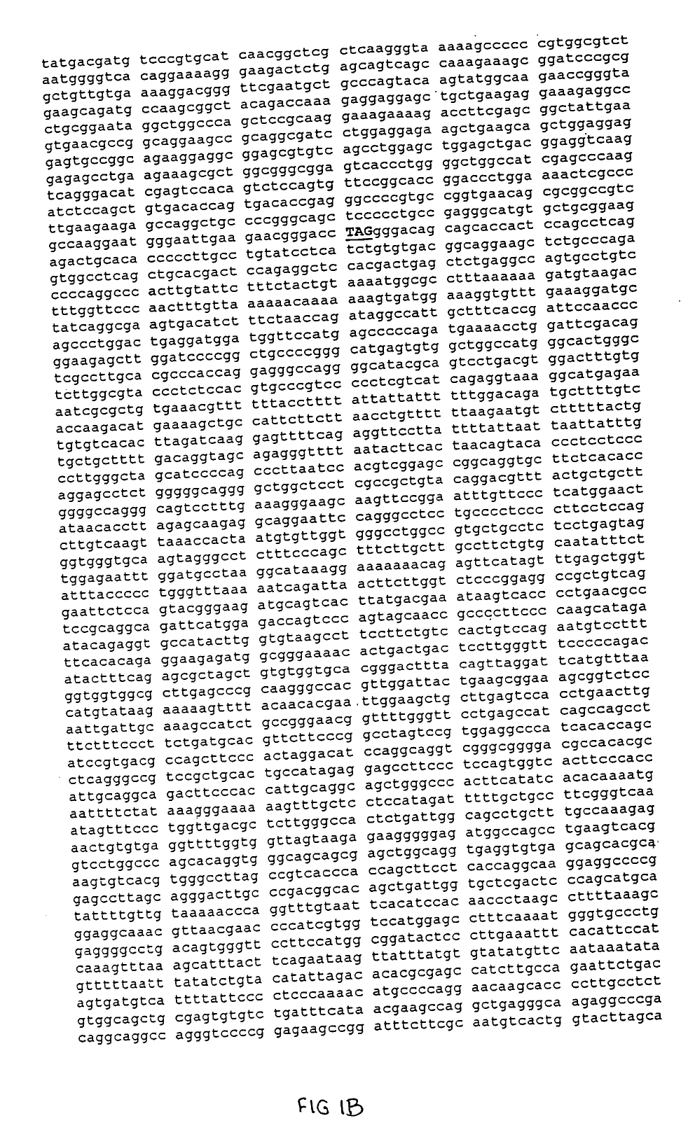

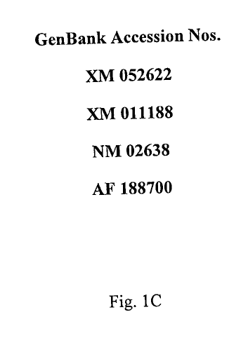

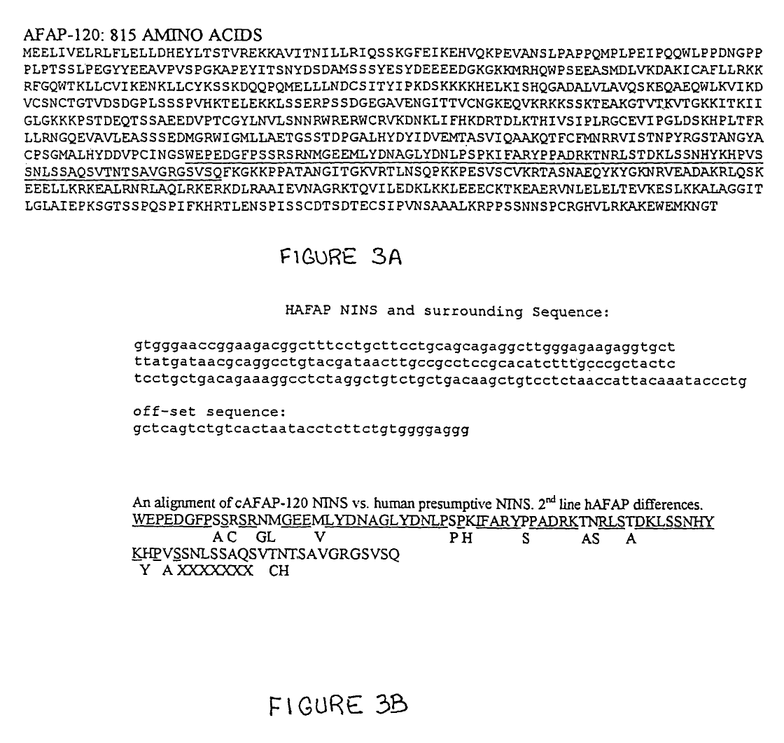

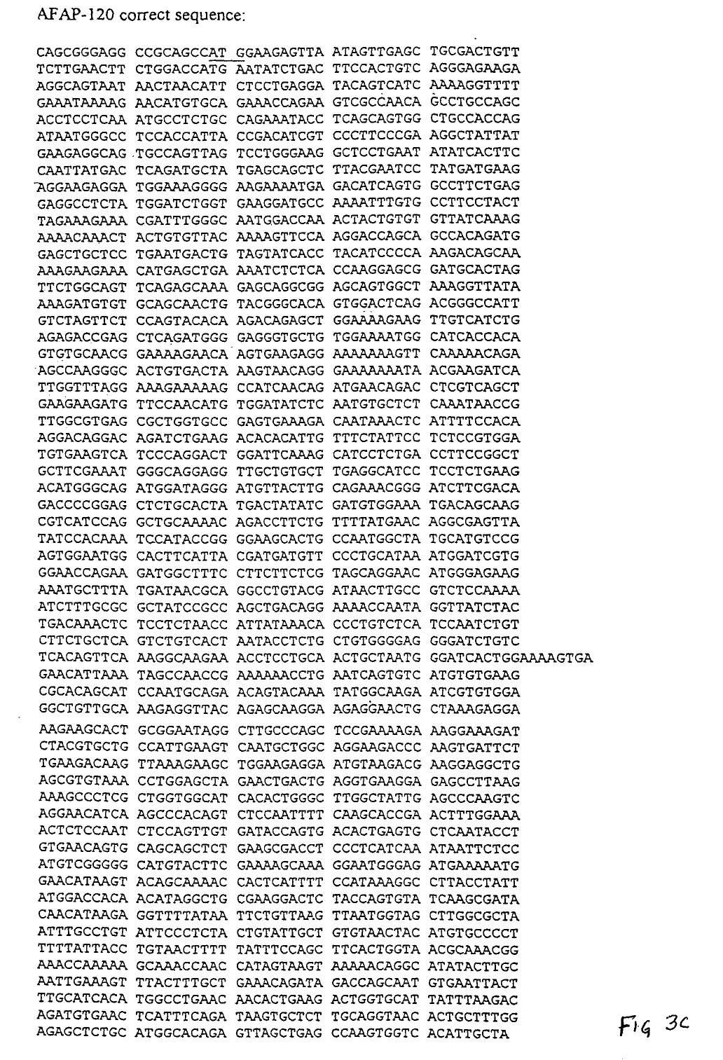

As used herein, a “variant” of an AFAP nucleic acid sequence comprises an AFAP nucleic acid sequence according to FIGS. 1A-C, FIG. 3C, or GenBank Accession Nos. L20303 or L20302, or a fragment thereof comprising one or more insertions, deletions, or substitutions. Preferably, the variant sequence specifically hybridizes to the nucleic acid sequence according to FIGS. 1A-C, FIG. 3C, or GenBank Accession Nos. L20303 or L20302, or is at least about 60% identical to an AFAP nucleic acid. In one aspect, a “variant” of an AFAP polypeptide sequence comprises greater than about 87% identity to the chicken AFAP polypeptide sequence disclosed in Flynn, et al., 1993, supra, as determined after maximally aligning sequences using methods routine in the art.

As used herein, a “modified form” of the AFAP polypeptide, fragment, or variant, refers to a post-translationally modified form of the polypeptide, fragment, or variant. Preferably, a “modified” AFAP polypeptide or variant comprises a phosphorylated AFAP polypeptide, fragment thereof, or variant thereof. A modification site in the polypeptide may be found in the native AFAP-110 or AFAP-120 polypeptide or can be generated after mutagenesis of the AFAP polypeptide, fragment, or variant thereof.

As used herein “isolated” refers to a nucleic acid substantially free of cellular material. An “isolated” nucleic acid is also substantially free of sequences which naturally flank the nucleic acid (i.e., sequences located at the 5′ and 3′ ends of the nucleic acid molecule) from which the nucleic acid is derived. The term “nucleic acid” is intended to include DNA and RNA and can be either double-stranded or single-stranded.

As used herein, “stringent conditions” refers to hybridization conditions and/or amplification conditions in which a probe or primer will specifically hybridize to a target nucleic acid while not binding substantially to non-target nucleic acids (i.e., less than about 5%, preferably, less than about 1%, of non-target nucleic acids). In one aspect, stringent hybridization conditions refers to 0.2×SSC or less at about 42° C. to 70° C., and preferably, at about 50° C. to 65° C. Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.).

As used herein, a “molecular probe” is any detectable molecule, or is a molecule which produces a detectable molecule upon reacting with a biological molecule (e.g., polypeptide or nucleic acid).

As used herein, “expression” refers to a level, form, or localization of product. For example, “expression of a protein” refers to one or more of the level, form (e.g., presence, absence or quantity of modifications, or cleavage or other processed products), or localization of the protein.

As used herein, “reporter molecule” refers to a nucleic acid or polypeptide product that is detectable when expressed by a cell comprising the molecule. Preferably, a reporter molecule allows a quantitative measurement of the expression of a nucleic acid sequence to which it is operably linked. Reporter polypeptides may be proteins capable of emitting light such as Green Fluorescent Protein (GFP) (Chalfie et al., 1994, Science 11: 263:802-805) or luciferase (Gould et al., 1988, Anal. Biochem. 15: 175: 5-13), or may be proteins which can catalyze a substrate (e.g., such as β-galactosidase). Reporter polypeptides also can be intracellular or cell surface proteins detectable by antibodies. Reporter molecules additionally, or alternatively, can be detected by virtue of a unique nucleic acid sequence not normally contained within the cell.

As used herein, “GFP” refers to a member of a family of naturally occurring fluorescent proteins, whose fluorescence is primarily in the green region of the spectrum. The term includes mutant forms of the protein with altered or enhanced spectral properties. Some of these mutant forms are described in Cormack, et al., 1996, Gene 173: 33-38 and Ormo, 1996, Science 273:1392-1395, the entireties of which are incorporated herein by reference. The term also includes polypeptide analogs, fragments or derivatives of GFP polypeptides which differ from naturally-occurring forms by the identity or location of one or more amino acid residues, (e.g., by deletion, substitution or insertion) and which share some or all of the properties of the naturally occurring forms so long as they generate detectable signals (e.g., fluorescence). Wild type GFP absorbs maximally at 395 nm and emits at 509 nm. High levels of GFP expression have been obtained in cells ranging from yeast to human cells. The term also includes Blue Fluorescent Protein (BFP), the coding sequence for which is described in Anderson, et al., 1996, Proc. Natl. Acad. Sci. USA 93:16, 8508-851 1, incorporated herein by reference.

As used herein, “regulatory element” refers to a genetic element which controls the expression of nucleic acid sequences. For example, a promoter is a regulatory element which directs the transcription of an mRNA. Other regulatory elements include enhancers and other transcription factor binding sites, splicing signals, polyadenylation signals, transcription termination signals, internal ribosome entry sites (IRES), etc.

As used herein, the term “operably linked” refers to functional linkage between a nucleic acid regulatory element (such as a promoter, and/or array of transcription factor binding sites) and a second nucleic acid sequence (such as a nucleic acid encoding a reporter polypeptide), where the regulatory element directs transcription of the nucleic acid corresponding to the second sequence.

As used herein, the term “selectable marker” refers to a gene which encodes an activity that confers on a cell comprising the gene the ability to grow in medium in which the cell would otherwise not survive. For example, the gene can encode an essential nutrient or be involved in the metabolism of an essential nutrient. Alternatively, a selectable marker may confer resistance to an antibiotic or drug or can convert a toxic product into a non-toxic product such that only cells comprising the gene are capable of surviving in the presence of the antibiotic, drug or toxic product.

As used herein, “transgene” means a nucleic acid sequence encoding an AFAP polypeptide or variant form thereof, which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can include one or more transcriptional regulatory sequences and other nucleic acid sequences such as introns, that may be necessary for optimal expression of a selected nucleic acid.

As defined herein, “a diagnostic trait” is an identifying characteristic, or set of characteristics, which in totality, are diagnostic. In one aspect, a trait is a marker for a particular cell type, such as a transformed, immortalized, pre-cancerous, or cancerous cell, or a state (e.g., a disease) and detection of the trait provides a reliable indicia that the sample comprises that cell type or state. Screening for an agent affecting a trait thus refers to identifying an agent which can cause a detectable change or response in that trait which is statistically significant within 95% confidence levels.

As used herein, the term “cancer” refers to a malignant disease caused or characterized by the proliferation of cells which have lost susceptibility to normal growth control. “Malignant disease” refers to a disease caused by cells that have gained the ability to invade either the cells of origin or to travel to sites removed from the cells of origin.

As used herein, a “cancer-specific marker” is a biomolecule which is expressed preferentially on cancer cells and is not expressed or is expressed to a small degree in non-cancer cells of an adult individual. As used herein, “a small degree” means that the difference in expression of the marker in cancer cells and non-cancer cells is large enough to be detected as a statistically significant difference when using routine statistical methods to within 95% confidence levels.

As used herein, “obesity” refers to the accumulation of excessive body fat. As used herein, an individual is “obese” if their weight is 20% or more over the midpoint of their weight range on a standard height-weight table. Obesity may be classified as mild (20-40% overweight), moderate (41-100% overweight) or severe (more than 100% overweight).

Data suggests a relationship between AFAP and both angiogenesis and obestity. Data presented herein demonstrate that AFAP relays signals from PKCα that stimulate Src signalling. Both PKCα and Src modulate angiogenesis (Spyridopoulos et al., 2002, Arterioscler Thromb Vasc Biol., 22:901-906; Teicher et al., 2001, In Vivo, 15:185-193; Yoshiji et al., 199, Cancer Res., 59:4413-4418; Lewis et al., 1996, J. Cell Biol., 134:1323-1332; Tsopanoglou et al., 1994, J. Vasc. Res., 31:195-204; Tsopanoglou et al., 1993, J. Vase. Res., 30:202-208). AFAP is expressed at high levels in endothelial cells in culture and in developing arteries and veins in chick and mouse. Since AFAP is also a binding partner for PKCβ1,as demonstrated herein, and a likely substrate, AFAP may relay signals from PKCβ1. A relationship between aberrant angiogenesis and the occurrence of obesity is suggested by the observations that fat pad cells require angiogenesis for nourishment and in mouse models bred for obesity, angiogenesis inhibitors reduce obesity (Rupnick et a., 2002, Proc., Natl. Acad. Sci. USA, 99:10730-10735).

As used herein, a “difference in expression” refers to an increase or decrease in expression. A difference may be an increase or a decrease in a quantitative measure (e.g., amount of a polypeptide or RNA encoding the polypeptide) or a change in a qualitative measure (e.g., a change in the localization of a polypeptide). Where a difference is observed in a quantitative measure, the difference according to the invention will be at least about 10% greater or less than the level in a normal standard sample. Where a difference is an increase, the increase may be as much as about 20%, 30%, 50%, 70%, 90%, 100% (2-fold) or more, up to and including about 5-fold, 10-fold, 20-fold, 50-fold or more. Where a difference is a decrease, the decrease may be as much as about 20%, 30%, 50%, 70%, 90%, 95%, 98%, 99% or even up to and including 100% (no specific polypeptide or RNA present). It should be noted that even qualitative differences may be represented in quantitative terms if desired. For example, a change in the intracellular localization of a polypeptide may be represented as a change in the percentage of cells showing the original localization.

As defined herein, the “efficacy of a drug” or the “efficacy of a therapeutic agent” is defined as the ability of the drug or therapeutic agent to restore the expression of a diagnostic trait to values not significantly different from normal (as determined by routine statistical methods, to within 95% confidence levels).

As defined herein “a sample” is a material suspected of comprising an analyte and includes a biological fluid, suspension, buffer, collection of cells, scraping, fragment or slice of tissue. A biological fluid includes blood, plasma, sputum, urine, cerebrospinal fluid, lavages, and leukapheresis samples.

As used herein, “pathway molecules” or “pathway biomolecules” are molecules involved in the same pathway and whose accumulation and/or activity and/or form (i.e., referred to collectively as the “expression” of a molecule) is dependent on other pathway molecules, or whose accumulation and/or activity and/or form affects the accumulation and/or activity or form of other pathway target molecules.

As used herein “a correlation” refers to a statistically significant relationship determined using routine statistical methods known in the art. For example, in one aspect, statistical significance is determined using a Student's unpaired t-test, considering differences as statistically significant at p<0.05.

As used herein a “diagnostic probe” is a probe whose binding to a tissue and/or cell sample provides an indication of the presence or absence of a particular trait. In one aspect, a probe is considered diagnostic if it binds to a diseased tissue and/or cell (“disease samples”) in at least about 80% of samples tested comprising diseased tissue/cells and binds to less than 10% of non-diseased tissue/cells in samples (“non-disease” samples). Preferably, the probe binds to at least about 90% or at least about 95% of disease samples and binds to less than about 5% or 1% of non-disease samples.

As used herein, the term “antibody” includes antibody fragments and single chain antibodies which specifically react with an AFAP polypeptide, fragment thereof, modified form thereof, or variant form thereof. Antibodies can be fragmented using conventional techniques and the fragments screened for utility as described above. For example, F(ab′)₂ fragments can be generated by treating antibody with pepsin. The resulting F(ab′)₂ fragment may be treated to reduce disulfide bridges to produce Fab′ fragments.

As used herein, a “hematopoietic stem cell” or “hematopoietic progenitor cell” or hematopoietic precursor cell” is one which is able to differentiate to form a more committed or mature blood cell type. For a review of hematopoiesis, see Dexter and Spooncer, 1987, Ann. Rev. Cell Biol. 3: 423-441.

As used herein, “lymphoid blood cell lineages” are those hematopoietic precursor cells which are able to differentiate to form lymphocytes (B-cells or T-cells).

As used herein, “erythroid blood cell lineages” are those hematopoietic precursor cells which are able to differentiate to form erythrocytes (red blood cells).

As used herein, “myeloid blood cell lineages”, encompasses all hematopoietic precursor cells, other than lymphoid and erythroid blood cell lineages as defined above.

AFAP Nucleic Acids

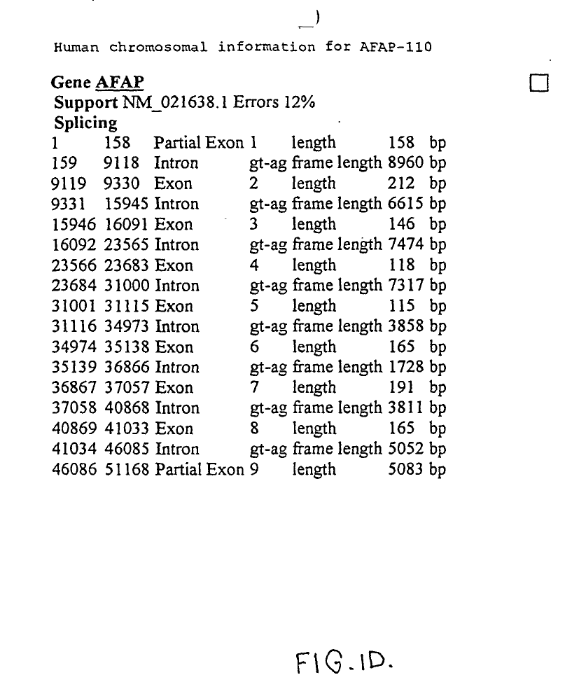

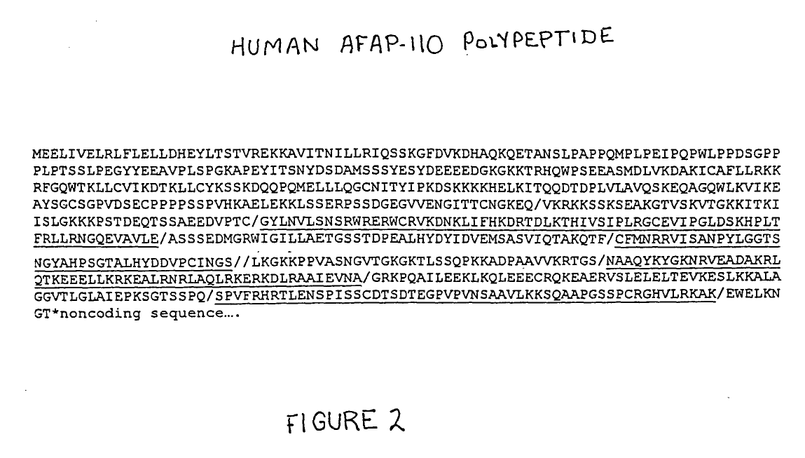

In one aspect, the invention provides nucleic acid molecules having substantial sequence identity or homology to the nucleic acid sequence as shown in FIGS. 1A-C or as shown in FIG. 3C or GenBank Accession Nos. L20303 or L20302, and nucleic acids encoding AFAP polypeptides having substantial identity or homology to the amino acid sequence shown in FIG. 2, FIG. 3A, or FIG. 3B. The invention also includes nucleic acid molecules encoding truncated AFAP polypeptides described further below, fusion polypeptides, and variant forms of such nucleic acids which arise by alternative splicing of an AFAP mRNA or which are generated by mutagenesis (arising through recombinant DNA techniques or which are naturally occurring allelic variants).

The method by which the nucleic acid is isolated is not limiting. For example, isolated nucleic acid molecules can be obtained from expressed sequence libraries (e.g., cDNA libraries) or genomic libraries using probes according to the invention, by amplification of cellular nucleic acids using primers according to the invention, by chemical synthesis, and by other methods routine in the art.

Modified nucleic acids also are encompassed within the scope of the invention, i.e., nucleic acids comprising one or more of modified bases, sugars, and internucleotide linkages which preferably have the substantially the same or enhanced stability and/or specificity for a target nucleic acid as the nucleic acids from which they are derived.

In one aspect, an isolated nucleic acid according to invention is RNA comprising the sequence as shown in FIGS. 1A-C, FIG. 3C, GenBank Accession Nos. L20303 or L20302, where the base T is substituted by a U. RNA can be isolated from total cellular mRNA by a variety of techniques, for example, by using the guanidinium-thiocyanate extraction procedure of Chirgwin et al., 1979, Biochemistry 18: 5294-5299. cDNA can then synthesized from the mRNA using reverse transcriptase (for example, Moloney MLV reverse transcriptase available from Gibco/BRL, Bethesda, Md.) and primers according to the invention.

In other aspects, an isolated nucleic acid molecule of the invention which is RNA can be obtained by cloning a cDNA encoding an AFAP polypeptide or fragment thereof into an appropriate vector which allows for transcription of the cDNA to produce an RNA molecule which encodes a protein which exhibits one or more AFAP activities (discussed further below). For example, a cDNA can be cloned downstream of a bacteriophage promoter, (e.g. a T7 promoter) in a vector, cDNA can be transcribed in vitro with a suitable polymerase (e.g., T7 polymerase), and the resultant RNA can be isolated by conventional techniques. In another aspect, the cDNA is a fragment of a full length cDNA which is of sufficient size to specifically hybridize to AFAP transcripts (preferably, human AFAP transcripts) under stringent conditions but which does not necessarily encode an AFAP polypeptide or fragment thereof.

In still other aspects, the invention contemplates providing nucleic acids encoding variants of AFAP polypeptides. Variants can include conservative changes in amino acid sequence or can include forms of AFAP polypeptides which lack or express one or more altered AFAP activities. These are discussed further below.

Preferably, a nucleic acid variant comprises at least about 60% nucleic acid sequence identity to AFAP nucleic acid sequences disclosed herein, while polypeptide variants comprise at least about 60% amino acid identity to AFAP amino acid sequences disclosed herein. Percent sequence identity or homology can be determined using methods routine in the art. For example, a sequence determined as having 60% sequence identity to an AFAP nucleic acid or polypeptide can be identified after maximally aligning the sequence to an AFAP nucleic acid or polypeptide sequences using any of a number of commercially available computer programs. A typical example of such a computer program is CLUSTAL. Other computer programs to determine identity and similarity between the two sequences include, but are not limited to, the GCG program package (Devereux et al., 1984, Nucleic Acids Research 12: 387), FASTA, and FASTP (Pearson, 1990, Methods Enzymol. 183: 63-98).

Preferably, a comparison method is selected which produces optimal alignments between a query sequence (e.g., a putative variant) and a target sequence (e.g., an AFAP sequence), taking into consideration possible insertions and deletions without penalizing unduly the overall identity score. This is achieved by inserting “gaps” in the sequence alignment to try to maximize local homology/identity. “Gap penalties” are assigned to each gap that occurs in the alignment so that for the same number of identical amino acids a sequence alignment with as few gaps as possible achieves a higher score than one with many gaps (i.e., reflecting higher relatedness between the two compared sequences). For example, when using the GCG Wisconsin Bestfit package the default gap penalty for amino acid sequences is −12 for a gap and −4 for each extension.

Calculation of maximum % identity generally requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12: 387). Examples of other software packages that can perform sequence comparisons include, but are not limited to, the BLAST package, FASTA and FASTP (Pearson, supra) and the GENEWORKS suite of comparison tools. Both BLAST and FASTA are available for offline and online searching.

BLAST (Basic Local Alignment Search Tool) is the heuristic search algorithm employed by the programs: blastp, blastn, blastx, tblastn, and tblastx; these programs ascribe significance to their findings using the statistical methods of Karlin and Altschul (Karlin and Altschul, 1990, Proc. NatL Acad. Sci. USA 87:2264-68; Karlin and Altschul, 1993, Proc. Natl. Acad. Sci. USA 90: 5873-7; see, http://www.ncbi.nih.gov/BLAST/blast_help.html) with a few enhancements. The BLAST programs are tailored for sequence similarity searching, for example to identify homologues to a query sequence. For a discussion of basic issues in similarity searching of sequence databases, see Altschul et al., 1994, Nature Genetics 6: 119-129.

A variant sequence with “substantial identity” to an AFAP sequence when assessed by BLAST equates to sequences which match with an EXPECT value of at least about 7, preferably at least about 9 and most preferably, 10 or more. The default threshold for EXPECT in BLAST searching is usually 10.

Probes and Primers

In one aspect, the invention provides one or more probes or primers which hybridize to nucleic acid molecules according to the invention and/or to one or more AFAP-encoding cellular transcripts or their complements and/or to genomic AFAP nucleic acids under stringent conditions.

As used herein, “stringent conditions” refers to hybridization conditions and/or amplification conditions in which a probe or primer will specifically hybridize to a target nucleic acid while not binding substantially to non-target nucleic acids (i.e., less than about 5%, preferably, less than about 1%, of non-target nucleic acids). In one aspect, stringent hybridization conditions refers to 0.2×SSC or less at, about 42° C. to 70° C., and preferably, at about 50° C. to 65° C. Appropriate stringency conditions which promote hybridization are known to those skilled in the art and are described, for example, Ausubel, 1989, in Current Protocols in Molecular Biology, John Wiley & Sons, N.Y. (eds), 6.3.1-6.3.6. Appropriate hybridization conditions under which primers specifically hybridize during amplification reactions (e.g., such as PCR and LCR) also are well known in the art.

In a preferred aspect, probes and primers according to the invention specifically hybridize to human chromosome 4q 16.1, the chromosomal site of the human gene encoding AFAP-110 and to isolated or partially isolated human genomic AFAP nucleic acids. Probes and primers according to the invention also are provided which specifically hybridize to AFAP transcripts (i.e., transcripts encoding AFAP-110 and/or AFAP-120 or other AFAP splice variants) or their complements. Preferably, the transcripts are human transcripts. In one aspect, a probe or primer comprising a sequence corresponding to a complement of the sequence encoding the amino acids shown in FIG. 3A or FIG. 3B corresponding to the neuron-specific NINS sequence of AFAP-120 or a fragment thereof of at least about 6, at least about 8, at least about 10, at least about 20, at least about 50, at least about 100, or at least about 200 nucleotides, such that the sequence or fragment thereof is capably of specifically identifying AFAP-120 transcripts and not AFAP-110 transcripts.

In one aspect, a nucleic acid molecule is provided which comprises greater than about 87% identity to a chicken AFAP-110 sequence.

In one aspect, probes and primers according to the invention are those which specifically hybridize to human AFAP nucleic acids and/or mouse nucleic acids but not to chicken AFAP nucleic acids.

A probe or primer may be labeled with a detectable substance and used to select from a mixture of nucleotide sequences, a nucleotide sequence coding for a protein which displays one or more of the properties of an AFAP polypeptide (e.g., actin binding and/or bundling, binding to Src, phosphorylation by a PKC polypeptide, and the like). In a preferred aspect, probe or primers are used to select a genomic AFAP nucleic acid sequence of about 6 nucleotides to about 500,000 nucleotides. In a preferred aspect, a an isolated genomic AFAP sequence is selected which specifically hybridizes to AFAP transcripts under stringent conditions.

The genomic sequence can include one or more regulatory sequences capable of driving the transcription of an AFAP mRNA molecule such as AFAP-110 and/or AFAP-120 in cells in which AFAP mRNA is normally expressed. However, in one aspect, genomic sequences comprising one or more AFAP regulatory sequences are provided which do not hybridize to AFAP transcripts. Such genomic sequences are preferably linked to a reporter gene, such as a gene encoding GFP.

Suitable labels include, but are not limited to, radioactive labels, antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, 1989, In Molecular Cloning: A Laboratory Manual, 2nd Edition, Cold Spring Harbor Laboratory Press, or can be immobilized on arrays as is known in the art. The support can be in the shape of: a tube, test plate, bead, disc, sphere, chip, wafer, capillary, and the like.

Nucleic Acids Encoding AFAP Polypeptides, Polypeptide Fragments and Variants

In one aspect, the invention provides nucleic acids encoding one or more domains of an AFAP polypeptide, preferably comprising at least one human AFAP polypeptide domain. For example, the invention provides a nucleic acid encoding truncated AFAP polypeptides (e.g., less than full-length polypeptides), such as a nucleic acid encoding one or more of a truncated polypeptide comprising an AFAP-110 WW binding domain; a truncated polypeptide comprising an AFAP-110 SH2 binding domain; a truncated polypeptide comprising an AFAP-110 SH3 binding domain; a truncated polypeptide comprising an AFAP-110 PH1 and/or PH2 domain; a truncated polypeptide comprising an AFAP-110 leucine zipper; a truncated polypeptide comprising an AFAP-110 actin-binding domain, a truncated polypeptide comprising an AFAP-110 serine/threonine kinase target region, and a truncated polypeptide comprising an AFAP-110 nuclear export domain. It is contemplated that domains within these truncated polypeptides can be separated by amino acids found in the naturally occurring AFAP polypeptide or can be separated by linker polypeptides fused in-frame to the domain sequences.

In other aspects, nucleic acids are provided which encode variant forms of AFAP polypeptides comprising conserved and/or non-conserved changes in AFAP amino acids. Such polypeptides are discussed further below. Nucleic acids encoding such polypeptides can be generated by site-directed mutagenesis using methods routine in the art or can arise naturally (e.g., as allelic variants) in populations.

Determination of whether a particular nucleic acid molecule encodes a protein having an AFAP activity or a desired altered activity (e.g., such as a nucleic acid encoding a truncated AFAP polypeptide or a variant AFAP polypeptide) can be accomplished by expressing an AFAP cDNA in an appropriate host cell by standard techniques, and testing the ability of the expressed protein to bind to perform the desired activity. In one aspect, an AFAP polypeptide or altered form thereof (i.e., truncated form or variant form) is at least partially purified from a host cell prior to testing its activit(ies). In another aspect, the activity of an AFAP polypeptide is determined by monitoring the phenotype of the cell (e.g., monitoring the actin bundling and/or the ability of the cell to acquire a cancer phenotype). A cDNA having a desired biological activity of an AFAP polypeptide can be sequenced by standard techniques, such as dideoxynucleotide chain termination or Maxam-Gilbert chemical sequencing, to determine the nucleic acid sequence and the predicted amino acid sequence of the encoded protein. Methods of testing for AFAP activities are described further below.

Nucleic Acids Encoding Fusion Polypeptides

The nucleic acid molecules of the invention may be inserted into an appropriate expression vector, i.e., a vector which contains the necessary elements for the transcription and translation of the inserted coding sequence. Accordingly, recombinant expression vectors adapted for transformation of a host cell may be constructed which comprise a nucleic acid molecule of the invention and one or more transcription and translation elements linked to the nucleic acid molecule. In one aspect, such vectors additionally comprise one or more selectable markers and/or an origin of replication enabling the vector to be replicated in at least one of the cell types into which it can be introduced.

Possible expression vectors include but are not limited to cosmids, plasmids, or modified viruses (e.g. replication defective retroviruses, adenoviruses and adeno-associated viruses), so long as the vector is compatible with the host cell used. In a particularly preferred aspect, an AFAP polypeptide is cloned into the pCMV-1 vector enabling its high expression in vertebrate cell lines such as chicken cell lines (e.g., such as the DF-1 cell line). In another aspect, the invention provides an avian specific retroviral vector (e.g., such as the RCAS vector) into which a nucleic acid encoding and AFAP polypeptide, fragment, or variant thereof has been cloned.

Suitable regulatory sequences may be derived from a variety of sources, including bacterial, fungal, viral, mammalian, or insect genes (For example, see the regulatory sequences described in Goeddel, 1990, Gene Expression Technology: Methods in Enzymology 185: Academic Press, San Diego, Calif.). Selection of appropriate regulatory sequences is dependent on the host cell chosen, and may be readily accomplished by one of ordinary skill in the art. The Regulatory sequences also can be supplied by probing vertebrate genomic libraries (preferably, human genomic libraries) using nucleic acid molecules according to the invention to identify native AFAP-110 regulatory sequences. Regulatory elements can be identified using standard techniques. The function of the elements can be confirmed by using these elements to express a reporter gene such as the lacZ gene or Green Fluorescent Protein (GFP) or other reporter gene which is operatively linked to one or more of the elements. These constructs may be introduced into cultured cells using conventional procedures or into non-human transgenic animal models. In addition to identifying regulatory elements in DNA, such constructs also be used to identify nuclear proteins interacting with the elements, using techniques known in the art.

Examples of selectable marker genes are genes encoding a protein such as G418 and hygromycin which confer resistance to certain drugs, .beta.-galactosidase, chloramphenicol acetyltransferase, firefly luciferase, or an immunoglobulin or portion thereof such as the Fc portion of an immunoglobulin preferably IgG. The selectable markers can be introduced on a separate vector from the nucleic acid of interest.

The recombinant expression vectors can also encode a fusion moiety which provides increased expression of the AFAP polypeptide; increased solubility of the AFAP polypeptide; and aid in the purification of AFAP polypeptide by acting as a ligand in affinity purification. For example, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMAL (New England Biolabs, Beverly, Mass.) and pRIT5 (Pharmacia, Piscataway, N.J.) which can be used to fuse glutathione S-transferase (GST), maltose E binding protein, or protein A, respectively, to the AFAP polypeptide.

Antisense Molecules

The invention further provides a recombinant expression vector comprising a DNA nucleic acid molecule of the invention cloned into an expression vector in an antisense orientation to provide for transcription of an RNA molecule which is antisense to the nucleotide sequence shown in FIGS. 1A-1C, FIG. 3C, nucleic acids identified as GenBank Accession Nos. L20303 or L20302, or a portion thereof sufficiently large to inhibit translation of an AFAP polypeptide. Regulatory sequences linked to the antisense nucleic acid can be chosen which direct the continuous expression of the antisense RNA molecule in a variety of cell types, for instance a viral promoter and/or enhancer, or regulatory sequences can be chosen which direct tissue or cell type specific expression of antisense RNA. Antisense nucleic acids can also be chemically synthesized and can be deoxynucleotides or modified forms thereof which are selected to have enhanced stability in vivo.

Techniques for generating antisense constructs are described in, for example, Stein et al., 1988, Cancer Research_{—}48: 2659-2668, 1988; Walder, 1988, Genes & Development 2: 502-504, Marcus-Sekura, 1988, Anal Biochemistry 172: 289-295; Zon, 1987, Journal of Protein Chemistry 6: 131-145; Zon, 1988, Pharmaceutical Research 5: 539-549; and Loose-Mitchell, 1988, TIPS 9: 45-47, the entireties of which are incorporated by reference. Antisense molecules according to the invention can comprise one or more modified nucleotides to enhance their stability (see, e.g., as described in Agrarwal et al., 1988, Proc. Natl. Acad. Sci. USA 85: 7079; Sarin et al., 1988, Proc. Natl. Acad. Sci. USA 85: 7448, for example, the entireties of which are incorporated herein by reference.

In a further aspect, the antisense molecule additionally comprises a catalytic activity and is able to cleave a target AFAP mRNA, thereby preventing the mRNA from being translated (see, e.g., Woolf, et al., 1992, Proc. Natl. Acad. Sci. USA 89: 7305-7309). Sequences with catalytic activity are described in, for example, Rossi et al., 1992, Aids Research and Human Retroviruses 8: 183, 1992; Hampel and Tritz, 1989, Biochemistry 28: 4929; and Hampel et al., 1990, Nucleic Acids Research 18: 299; Perrotta et al., 1992, Biochemistry 31: 16; Guerrier-Takadaet al., 1989, Cell 35: 849; Collins et al., 1990, Cell 61: 685-696; Collins and Olive, 1983, Biochemistry 32: 2795-2799; Scanlon et al., 1991, Proc. Natl. Acad. Sci. USA 88: 10591-5, 1991; Dropulic et al., 1992, J. Virol. 66: 1432-41, Weerasinghe, et al., 1991, J. Virol. 65: 5531-5534; Ojwang et al., 1992, Proc. Natl. Acad. Sci. USA 89: 10802-10806; Chen et al., 1992, Nucleic Acids Res. 20: 4581-1589; Sarver et al., 1992, Science 247: 1222-1225; the entireties of which are incorporated herein by reference.

Targeting Molecules

In one aspect, the nucleic acids molecules according to the invention are complexed to targeting molecules which bind to specific cells to deliver the nucleic acid molecules to such cells. The targeting molecule can be complexed indirectly to the nucleic acid molecules (e.g., by coupling the targeting molecule to a lipid particle or viral particle comprising the nucleic acid molecule) or can be coupled directly to the nucleic acid molecule (e.g., via complementary or “sticky-end” binding to a 5′ or 3′ terminal of the nucleic acid or via a chemical linkage) using methods routine in the art.

In one aspect, the targeting molecule is a ligand for a receptor or protein specifically or preferentially expressed on diseased cells. For example, the targeting molecule can specifically bind to a polypeptide expressed specifically or preferentially on abnormally proliferating cells, such as cancer cells or adipocytes of an abnormal size (for example adipocytes that are 5% or more larger in size than an average-sized adipocyte). In another aspect, the targeting molecule recognizes a cell type in which an AFAP polypeptide is normally expressed (e.g., such as neural cells, hematopoietic cells or brain cells) as a means of targeting nucleic acid molecules encoding functional AFAP polypeptides to cell(s) in need of such polypeptides. In a further aspect, the targeting molecule delivers AFAP antisense nucleic acids to cells which overexpress or ectopically express AFAP polypeptides as a means of reducing or eliminating the expression of these polypeptides.

AFAP Polypeptides

The recombinant expression vectors described above can be used to prepare transformed host cells expressing an AFAP polypeptide. In one aspect, such cells are used to produce a substantially purified AFAP polypeptide, fragment thereof, fusion protein thereof, or variant form thereof. For example, an expression vector as described above can be introduced into a host cell and host cells containing the expression vector can be cultured under conditions which allow for the expression of the polypeptide, fragment thereof, fusion thereof, or variant thereof. Preferably, the polypeptide, fragment thereof, fusion thereof, or variant thereof, is substantially isolated from other cellular biomolecules (e.g., by an affinity-based purification method as is known in the art). Preferably, the host cells containing the expression vector are selected and are substantially isolated from cells not containing the expression vector (e.g., by cloning).

An expression vector can be introduced into prokaryotic cells by electroporation or by transformation mediated by a chemical agent such as calcium-chloride or rubidium chloride. Nucleic acids can be introduced into mammalian cells by conventional techniques such as by calcium phosphate or calcium chloride co-precipitation, DEAE-dextran-mediated transfection, lipofectin, electroporation or microinjection. Suitable methods for transforming and transfecting host cells can be found in Sambrook et al., 1989, supra, for example.

The proteins of the invention may also be prepared by chemical synthesis using techniques well known in the art such as solid phase synthesis (Merrifield, 1964, J. Am. Chem. Assoc. 85: 2149-2154) or synthesis in homogenous solution (Houbenweyl, 1987, Methods of Organic Chemistry, ed. E. Wansch, Vol. 15 I and II, Thieme, Stuttgart).

The AFAP polypeptides of the invention may be conjugated with other molecules, such as non-AFAP polypeptides, to prepare fusion proteins. This may be accomplished, for example, by the synthesis of N-terminal or C-terminal fusion proteins. N-terminal or C-terminal fusion proteins can comprise a desired portion of an AFAP polypeptide (e.g., comprising one or more AFAP domains and/or encoding NINS sequences) fused to the sequence of a selected polypeptide with a desired biological function and/or which can be used to identify cells expressing recombinant AFAP sequences. Examples of selected polypeptides which may be used to prepare fusion proteins include immunoglobulins, glutathione-S-transferase (GST), hemagglutinin (HA), β-galactosidase, truncated myc, and Green Fluorescent Protein (GFP).

In one aspect, truncated AFAP polypeptides are provided. Preferably, such polypeptides comprise at least one human AFAP polypeptide domain, e.g., a truncated polypeptide comprising one or more of: an AFAP-110 SH3 binding motif, a WW binding domain, an AFAP-110 SH2 binding domain; an AFAP-110 PH1 and/or PH2 domain, an H-SH2 domain, an AFAP-110 leucine zipper, an AFAP-110 actin-binding domain, an AFAP-110 serine/threonine kinase target domain, and an AFAP-110 nuclear export domain.

The polypeptides of the invention also may also include variant AFAP polypeptides containing one or more amino acid substitutions, insertions, and/or deletions. Amino acid substitutions may be of a conserved or non-conserved nature. Conserved amino acid substitutions involve replacing one or more amino acids of the AFAP amino acid sequences shown in FIG. 2 or FIGS. 3A and 3B with amino acids of similar charge, size, and/or hydrophobicity characteristics. Preferably, such variants retain substantially all of the activity of native AFAP polypeptides. Non-conserved substitutions involve replacing one or more amino acids of the AFAP amino acid sequences with one or more amino acids which possess dissimilar charge, size, and/or hydrophobicity characteristics. In one aspect, amino acid insertions, substitutions and/or deletions may be introduced into one or more AFAP domains to produce variant polypeptides according to the invention which lack or show altered function of the one or more domains. In another aspect, an AFAP variant polypeptide is a polypeptide is constitutively activated. In a further aspect, an AFAP variant polypeptide is a polypeptide which is constitutively inactivated. Truncated AFAP variant polypeptides also can be provided.

In a preferred aspect, an AFAP variant polypeptide is provided which comprises one or more deletions, insertions, and/or substitutions in a AFAP PH domain.

In one aspect, an AFAP polypeptide is provided which comprises a deletion of the 2^{nd} to 4^{th} beta sheet of an amino terminal AFAP-110 PH domain (PH1). Such a variant AFAP polypeptide shows altered interactions with PKC.

In another aspect, an AFAP polypeptide is provided which comprises a deletion of the 5th to 7^{th} beta sheet of an amino terminal AFAP-110 PH1 domain. Such a variant AFAP polypeptide shows altered interactions with PKC.

In still another aspect, an AFAP polypeptide is provided comprising one or more basic residues and/or tryptophan in the carboxy-terminal PH domain substituted by one or more other amino acid and/or deleted for one or more of these amino acids. In a preferred aspect, the AFAP polypeptide substantially fails to bind phosphoinositide. In another preferred aspect, the AFAP variant polypeptide substantially fails to localize to the cell membrane.

In a further aspect, an AFAP variant polypeptide comprises a deletion of an AFAP leucine zipper domain. Alternatively, the AFAP polypeptide can comprise one or more deletions, insertions, and/or substitutions within this domain.

In a preferred aspect, an AFAP variant polypeptide is provided which comprises one or more deletions, insertions, and/or substitutions in an AFAP SH3 domain.

In another aspect, an AFAP variant polypeptide is provided which comprises one or more deletions, insertions, and/or substitutions in an AFAP SH2 domain (either or both of the AFAP SH2 domains).

In still another aspect, an AFAP variant polypeptide is provided which comprises one or more deletions, insertions, and/or substitutions in an AFAP WW domain.

In still another aspect, an AFAP variant polypeptide is provided which comprises one or more deletions, insertions, and/or substitutions in an AFAP actin-binding domain.

In a further aspect, an AFAP variant polypeptide is provided which comprises one or more deletions, insertions, and/or substitutions in an AFAP serine/threonine kinase target domain.

In still a further aspect, an AFAP variant polypeptide is provided which comprises one or more deletions, insertions, and/or substitutions in an AFAP nuclear export domain.

In a further aspect, a variant AFAP polypeptide is provided which comprises one or more insertions, deletions, and/or substitutions in the NINS amino acid sequence. The invention also provides an AFAP polypeptide comprising one or more NINS domains. In one aspect, the AFAP polypeptide is a truncated AFAP polypeptide.

The invention also contemplates chimeric forms of variant AFAP polypeptides (e.g., comprising variations in a plurality of AFAP domains). AFAP domains derived from different species also are provided. For example, in one aspect, AFAP polypeptides are provided which comprise two or more of: a human AFAP domain, a mouse AFAP domain and a chicken AFAP domain.

Phosphorylated or activated AFAP polypeptides of the invention also are provided. For example, phosphorylation may be induced by infecting bacteria harboring a plasmid containing a nucleotide sequence of the invention or fragment thereof, with a bacteriophage encoding the cytoplasmic domain of a suitable kinase (e.g., a Src family kinase), preferably, fused to a reporter polypeptide. Bacteria containing the plasmid and bacteriophage as a lysogen are isolated. Following induction of the lysogen, the expressed peptide becomes phosphorylated by the kinase.

In a particularly preferred embodiment, a dual expression vector is provided which expresses both an AFAP polypeptide and an AFAP binding protein (e.g., such as Src). The expression vector is introduced into a cell line lacking both AFAP and Src, such as a mammalian cell line (e.g., COS-1 AFAP-, Src-cell line) or avian cell line (e.g., such as a chicken AFAP-, Src-cell line) and the phosphorylation of AFAP by Src is monitored (see, e.g., as described in Guappone et al., 1996, Methods in Cell Science 18: 55-65, the entirety of which is incorporated by reference herein.

AFAP polypeptides, fragments thereof, modified forms thereof, and variants thereof, can be stably associated with a solid support. Suitable supports include, but are not limited to, agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, silicon, glass, etc. The support can be in the shape of, a tube, test plate, bead, disc, sphere, chip, wafer, capillary, etc. Polypeptides, fragments thereof, modified forms thereof, and variants thereof, stably associated with solid supports can be prepared by reacting these molecules with a suitable support using known chemical methods (e.g., covalent or ionic bonding) using for reactive molecules on the support or by physical methods (such as adsorption).

One or more AFAP activities can be monitored to evaluate the function of the AFAP polypeptide fragments and/or variants generated. In one aspect, an AFAP activity is the ability to form a complex with activated forms of Src, such as vSrc or Src^{527F} and/or other Src family kinases. Such an activity can be measured by co-immunoprecipitating polypeptide with anti-phosphotyrosine antibodies by Western blot analysis as described in Reynolds et al., 1989, Mol. Cell. Biol. 9: 3951-3958, for example. Activation of Src also can be measured either directly (e.g., by detecting or quantitating phosphorylated forms of Src, such as by using the phospho-Y416 antibody) or by measuring activation of downstream Src pathway proteins such as Rho GTPase (see, e.g., Fincham et al., 1999, J Cell. Sci., 112: 947-956).

In one aspect, AFAP activity measured is phosphorylation of an AFAP polypeptide. In one aspect, phosphorylation at one or more amino acids corresponding to amino acids 200-400, preferably amino acids 250-350, in the human AFAP-110 is determined. In a preferred aspect, phosphorylation at an Akt phosphorylation site is determined. Preferably, this site is with the AFAP-110 serine/threonine kinase target domain. Still more preferably, phosphorlyation at Threonine 337 is monitored. AFAP phosphorylation by Src and/or by a PKC polypeptide and/or by Ser/Thr kinases also can be determined using assays known in the art.

The ability of the AFAP polypeptide, fragment, modified form, or variant, to co-assemble with stress filaments and the cell membrane also can be measured using an assay such as described in Kanner et al., EMBO J. 10: 1689-1698, for example. In another aspect, transformation of cells comprising AFAP polypeptide, fragment, modified form, or variant can be monitored (see, e.g., Felice et al., 1990, supra). Actin binding ability and/or actin bundling ability also can be detected (e.g., using low speed co-sedimentation assays since at 20,000 g, actin filaments fail to pellet). In still a further aspect, cell motility is measured.

In another aspect, the ability of the AFAP polypeptide, fragment, modified form, or variant, to multimerize with other AFAP polypeptides (using the assay described in Qian et al., 1998, Oncogene 16: 2185-2195, for example, or a gel filtration assay) and/or the inability to multimerize in the presence of activated Src is determined. In a further aspect, the ability to bind to phospholipids (e.g., via one or more PH domains) is monitored (see, e.g., assays described in Gray et al., 1999, for example) or to bind to a PH domain binding protein (e.g., PKC isoforms, WD40-repeat containing proteins such as RACK I and/or Ggb, phospholipid second messengers). In still a further assay, the ability of the AFAP polypeptide, fragment, modified form, or variant, to localize to the cell membrane in the presence or absence of phosphoinositide is monitored. The ability of an AFAP polypeptide, fragment, modified form, or variant to bind to one or more of Lyn, Fyn, cYes also can be detected.

It should be obvious to those of skill in the art that as new activities of AFAP are appreciated, these may be monitored as a means to evaluate the function of the polypeptides described herein.

Antibodies Which Recognize AFAP Polypeptides

The invention further contemplates antibodies having specificity against an epitope of an AFAP polypeptide, fragments thereof, modified forms thereof, and/or variant forms thereof. Antibodies may be labeled with a detectable marker and used to detect AFAP polypeptides in tissues and cells and/or can be used to modulate the phenotype of these tissues/cells.

AFAP polypeptides, fragments thereof, modified forms thereof, and variants thereof, can be used to prepare antibodies specific for these biomolecules. Antibodies can be prepared which bind a distinct epitope in an unconserved region of the human AFAP polypeptide enabling such antibodies to specifically recognize human AFAP polypeptides and not to recognize other forms (e.g., chicken AFAP); although antibodies which bind to conserved regions also are contemplated. Antibodies can be raised against fusion proteins created by expressing fusion proteins in host cells as described above or can be raised against chemically synthesized peptides or polypeptides which additionally can be conjugated to other molecules, e.g., such as haptens.

Polyclonal antisera or monoclonal antibodies can be made using methods known in the art. A mammal such as a mouse, hamster, or rabbit, can be immunized with an immunogenic form of an AFAP polypeptide, fragment, modified form thereof, or variant form thereof. Techniques for conferring immunogenicity on such molecules include conjugation to carriers or other techniques well known in the art. For example, the immunogenic molecule can be administered in the presence of adjuvant. Immunization can be monitored by detection of antibody titers in plasma or serum. Standard immunoassay procedures can be used with the immunogen as antigen to assess the levels and the specificity of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art (see, e.g., Kohler and Milstein, 1975, Nature 256: 495-497; Kozbor et al., 1983, Immunol. Today 4: 72, Cole et al., 1985, In Monoclonal Antibodies in Cancer Therapy, Allen R. Bliss, Inc., pages 77-96). Additionally, techniques described for the production of single-chain antibodies (U.S. Pat. No. 4,946,778) can be adapted to produce antibodies according to the invention.

Antibody fragments which contain specifically bind to AFAP polypeptides, fragments thereof, modified forms thereof, and variants thereof, also may be generated by known techniques. For example, such fragments include, but are not limited to, F(ab′)₂ fragments which can be produced by pepsin digestion of the antibody molecule and the Fab fragments which can be generated by reducing the disulfide bridges of the F(ab′)₂ fragments. VH regions and FV regions can be expressed in bacteria using phage expression libraries (e.g., Ward et al., 1989, Nature 341: 544-546; Huse et al., 1989, Science 246: 1275-1281; McCafferty et al., 1990, Nature 348: 552-554).

Chimeric antibodies, i.e., antibody molecules that combine a non-human animal variable region and a human constant region also are within the scope of the invention. Chimeric antibody molecules include, for example, the antigen binding domain from an antibody of a mouse, rat, or other species, with human constant regions. Standard methods may be used to make chimeric antibodies containing the immunoglobulin variable region which recognizes the gene product of AFAP antigens of the invention (see, e.g., Morrison et al., 1985, Proc. Natl. Acad. Sci. USA 81: 6851; Takeda et al., 1985, Nature 314: 452; U.S. Pat. No. 4,816,567; U.S. Pat. No. 4,816,397). Chimeric antibodies are preferred where being used therapeutically to treat a condition associated with physiological responses to an aberrant AFAP signaling pathway.

Monoclonal or chimeric antibodies can be humanized further by producing human constant region chimeras, in which parts of the variable regions, particularly the conserved framework regions of the antigen-binding domain, are of human origin and only the hypervariable regions are of non-human origin. Such immunoglobulin molecules may be made by techniques known in the art, (e.g., Teng et al., 1983, Proc. Natl. Acad. Sci. USA 80: 7308-7312; Kozbor et al., 1983, Immunology Today 4: 7279; Olsson et al., 1982, Meth. Enzymol. 92: 3-16; WO 92/06193; EP 0239400).

In a particularly preferred aspect, an antibody is provided which recognizes a modified and/or variant form of an AFAP polypeptide but which does not recognize a non-modified and/or non-variant form of the AFAP polypeptide. For example, peptides comprising the variant region of a variant polypeptide can be used as antigens to screen for antibodies specific for these variants. Similarly modified peptides or proteins can be used as immunogens to select antibodies which bind only to the modified form of the protein and not to the unmodified form. Methods of making variant-specific antibodies and modification-specific antibodies are known in the art and described in U.S. Pat. No. 6,054,273; U.S. Pat. No. 6,054,273; U.S. Pat. No. 6,037,135; U.S. Pat. No. 6,022,683; U.S. Pat. No. 5,702,890; U.S. Pat. No. 5,702,890, and in Sutton et al., J. Immunogenet 14(1): 43-57 (1987), for example, the entireties of which are incorporated by reference herein.

In one aspect, an antibody is provided which specifically recognizes AFAP-120 and not AFAP-110. Preferably, the antibody recognizes an epitope within the NINS amino acid sequence of the AFAP-120 polypeptide shown in FIG. 3A or 3B.

In another aspect, an antibody directed against amino acids 1-55 of the human AFAP-110 sequence is provided.

In further aspects, antibodies which specifically recognize a human AFAP SH3 binding motif; a WW binding domain; an AFAP-110 SH2 binding domain; an AFAP-110 PH1 and/or PH2 domain, an H-SH2 domain, an AFAP-110 leucine zipper, an AFAP-110 actin-binding domain, a serine/threonine kinase target domain, and an AFAP-110 nuclear export domain are provided or variant or modified forms of these domains.

In one aspect, labeled antibodies or antigen-binding portions thereof are provided. Antibodies can be labeled with a fluorescent compound such as fluorescein, amino coumarin acetic acid, tetramethylrhodamine isothiocyanate (TRITC), Texas Red, Cy3.0 and Cy5.0. GFP is also useful for fluorescent labeling, and can be used to label non-antibody protein probes of AFAP (e.g., Src) as well as antibodies or antigen-binding portions thereof by expression as fusion proteins. GFP-encoding vectors designed for the creation of fusion proteins are commercially available. Other labels include, but are not limited to, alkaline phosphatase, beta-galactosidase, or acetylcholinesterase; luminescent materials such as luminol; radioactive materials, electron dense substances, such as ferritin or colloidal gold, and other molecules such as biotin.

Additional Antibodies Useful According to the Invention

Additional antibodies useful according to the invention include but are not limited to the following. PKC isoform antibodies (PKC sampler kit) and phosphotyrosine antibody are available from Transduction Laboratories. PKCζ antibody is available from Calbiochem. The polyclonal pan-PKC antibody is available from Calbiochem (Ab-1). AFAP-110 antibodies 4C3 and F1 were generated and characterized as previously described (Kanner et al., 1989; Kanner et al., 1990; Flynn et al. 1993; Qian et al., 1999). Phosphatidylserine, rhodamine-phalloidin, diolein and anti-Flag antibody are available from Sigma. Anti-GFP is available from Zymed. Anti-phospho-Src (Y⁴¹⁶) is available from Upstate Biotechnology or Cell Signaling (MA). Antiphosphotyrosine antibody is available from BD Transduction Labs. Anti-rabbit Alexa 633 and anti-mouse Alexa 488 are available from Molecular Probes. Anti-HA Tag antibody is available from Santa Cruz Biotechnology.

Host Cells

The invention further provides host cells containing any of the nucleic acids described above. Suitable host cells include prokaryotic and eukaryotic host cells. For example, suitable host cells include, but are not limited to: bacterial cells such as E. coli, insect cells (e.g., using baculovirus), yeast cells or mammalian cells.

Hybridoma cells can be screened immunochemically for production of antibodies specifically reactive with the immunogens described above. Therefore, the invention also contemplates hybridoma cells secreting monoclonal antibodies with specificity for AFAP polypeptides, fragments thereof, modified forms thereof, and variants thereof described above.

In a currently preferred embodiment an avian cell line is provided which expresses a recombinant AFAP polypeptide, fragment thereof, modified form thereof, or variant form thereof. Preferably, the avian cell line is an immortalized cell line such as the chicken DF-1 cell line.

Alternatively, the proteins of the invention also be expressed in non-human transgenic animals such as, rats, rabbits, sheep, pigs, and non-human primates (see, e.g., Hammer et al., 1995, Nature 315: 680-683; Palmiter et al., Science 222: 809-814, 1983; Brinster et al., 1985; Proc. Natl. Acad. Sci. USA 82: 4438-4442; Palmiter and Brinster, 1985, Cell 41: 343-345; U.S. Pat. No. 4,736,866).

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Where in vitro fertilization is used, the fertilized embryo may be implanted into a surrogate host or incubated in vitro, or both. Progeny may be evaluated for the presence of the transgene using methods routine in the art. Both heterozygous and homozygous animals are encompassed within the scope of the invention as are chimeric animals.

The invention contemplates providing cells, tissues, and non-human animals comprising disrupted or variant AFAP polypeptides. Null alleles may be generated in cells, such as embryonic stem (ES) cells by gene disruption. An AFAP gene also may be engineered to contain an insertion mutation which inactivates AFAP. Such a “knockout construct” may then be introduced into a cell, such as an ES cell, by a technique such as transfection, electroporation, injection, etc. Cells lacking an intact AFAP gene may then be identified, for example by Southern blotting, Northern Blotting or by assaying for expression of AFAP using the methods described herein. Such cells may then be fused to embryonic cells to generate transgenic non-human animals deficient in AFAP (e.g., “AFAP knockouts”). Germline transmission of the mutation may be achieved, for example, by aggregating the embryonic stem cells with early stage embryos, such as 8 cell embryos, in vitro; transferring the resulting blastocysts into recipient females and; generating germline transmission of the resulting aggregation chimeras. Such a mutant animal may be used to define specific cell populations, developmental patterns and in vivo processes, normally dependent on AFAP expression.

Generally, the embryonic stem cells (ES cells) used to produce such knockout animals will be of the same species as the knockout animal to be generated. For example, mouse embryonic stem cells usually will be used for generation of knockout mice. Embryonic stem cells can be generated and maintained using methods well known to the skilled artisan such as those described by Doetschman et al., 1985, J. Embryol. Exp. Morphol. 87: 27-45.

Any line of ES cells can be used; however, the line chosen is typically selected for the ability of the cells to integrate into and become part of the germ line of a developing embryo so as to create germ line transmission of the knockout construct. One mouse strain that is typically used for production of ES cells is the 129J strain. Other ES cell lines include, but are not limited to the murine cell line D3 (American Type Culture Collection, catalog no. CKL 1934) and the WW6 cell line (loffe et al., 1995, Proc. Natl. Acad. Sci. 92: 7357-7361). The cells are cultured and prepared for insertion of a knockout construct (e.g., a construct comprising AFAP nucleic acids) using methods routine in the art (see, e.g., Robertson, 1987, In Teratocarcinomas and Embryonic Stem Cells: A Practical Approach, E. J. Robertson, ed., IRL Press, Washington, D.C.; Bradley et al., 1986, Current Topics in Devel. Biol. 20: 357-371; Hogan et al., 1986, In Manipulating the Mouse Embryo: A Laboratory Manual, Cold Spring Harbor Laboratory Press, Cold Spring Harbor, N.Y.). Non-mouse embryonic stem cells are known and also are encompassed within the scope of the invention (see, e.g., Talbot et al., 2001, Anat. Rec. 264(1): 101-13 (pig); Chen et al., 1999, Theriogenology 52(2): 195-212 (pig); Iwazaki et al., 2000, Biol. Reprod. 62(2): 470-5 (cow); Schoonjans et al., 1996, Mol Reprod Dev. 45(4): 439-43 (rabbit); Li et al., 2001, Blood 98(2): 335-42 (monkey); Marshall et al., 2001, Methods Mol. Biol. 158: 11-8 (monkey)).

Insertion of a knockout construct into ES cells can be accomplished using a variety of methods well known in the art including, for example, electroporation, microinjection, and calcium phosphate treatment. A preferred method of insertion is electroporation. More than one type of construct can be introduced into an ES cell, simultaneously or sequentially.

Screening for ES cells comprising an AFAP knockout construct can be accomplished using a variety of methods. In one aspect, integration of a disrupted AFAP nucleic acid at a homologous site in the genome (e.g., the site of the AFAP gene) results in the integration of a marker gene which is part of the knockout construct (e.g., the marker is inserted into an AFAP coding region). The marker gene may encode a product enabling survival in a selection medium, such that any surviving ES cells are those which have integrated the knockout construct. The marker gene also can be a gene that encodes an enzyme whose activity can be detected (e.g., β-galactosidase) and the enzymatic activity of the cells can be analyzed. Where the marker gene does not confer a detectable phenotype, southern blots of ES cell genomic DNA can be probed with a sequence of DNA designed to hybridize only to the marker sequence. Alternatively, PCR can be used.

The integration of a marker gene also can be used to distinguish between ES cells in which the AFAP nucleic acid portion of the knockout construct has integrated by homologous recombination and those in which the AFAP nucleic acid has integrated by non-homologous recombination, such that loss of the marker can be used to identify ES cells in which desired homologous recombination events have occurred (i.e., replacing the endogenous AFAP gene with the disrupted AFAP nucleic acid). Other methods for determining the site of integration of the AFAP nucleic acid also can be used. For example, ES cell total DNA can be extracted from and the DNA can be probed on a Southern blot with a probe or probes designed to hybridize in a specific pattern to genomic DNA digested with particular restriction enzyme(s). Alternatively, or additionally, the genomic DNA can be amplified by PCR with probes specifically designed to amplify DNA fragments of a particular size and sequence (i.e., such that only those cells containing the knockout construct in the proper position will generate DNA fragments of the proper size).

After suitable ES cells containing the knockout construct in the proper location have been identified, the cells can be inserted into an embryo, preferably by microinjection. For microinjection, about 10-30 cells are collected into a micropipet and injected into embryos that are at the proper stage of development (e.g., such as a blastocyst) to permit integration of the foreign ES cell containing the knockout construct into the developing embryo. The suitable stage of development for the embryo used for insertion of ES cells is very species dependent, however for mice it is about 3.5 days. The embryos are obtained by perfusing the uterus of pregnant females (see, e.g., as described in Bradley et al., supra).

While any embryo of the right stage of development is suitable for use, preferred embryos are male. In mice, the preferred embryos also have genes coding for a coat color that is different from the coat color encoded by the ES cell genome. In this way, the offspring can be screened easily for the presence of the knockout construct by looking for mosaic coat color (indicating that the ES cell was incorporated into the developing embryo). Thus, for example, if the ES cell line carries the genes for white fur, the embryo selected will carry genes for black or brown fur.

After the ES cell has been introduced into the embryo, the embryo may be implanted into the uterus of a pseudopregnant foster mother for gestation. While any foster mother may be used, the foster mother is typically selected for her ability to breed and reproduce well, and for her ability to care for the young. Such foster mothers are typically prepared by mating with vasectomized males of the same species. The stage of the pseudopregnant foster mother is important for successful implantation, and it is species dependent. For mice, this stage is about 2-3 days pseudopregnant.

Offspring that are born to the foster mother may be screened initially for mosaic coat color where the coat color selection strategy has been employed. DNA from tissue of the offspring (e.g., tail or ear) may be screened for the presence of the knockout construct using Southern blots and/or PCR as described above. Offspring that appear to be mosaics may then be crossed to each other if they are believed to carry the knockout construct in their germ line in order to generate homozygous knockout animals. Homozygotes may be identified by Southern blotting of equivalent amounts of genomic DNA from mice that are the product of this cross, using DNA from known heterozygotes and wild type mice as controls.

Northern blots also can be used to probe mRNA of candidate knockout animals for the presence or absence of transcripts encoding either the gene knocked out, the marker gene, or both. In addition, Western blots can be used to assess the level of expression of AFAP mRNA and/or polypeptides in tissues of the animal by probing Western blots or tissue microarrays comprising samples from such animals with an antibody which recognizes AFAP polypeptides or an antibody against a marker gene product, where this gene is expressed. Fluorescence activated cell sorting or FACs analysis of various cells from the offspring also can be conducted using suitable antibodies to look for the presence or absence of AFAP polypeptides.

Animals containing more than one knockout construct and/or more than one transgene construct can be prepared in any of several ways. In one aspect, a series of animals, each containing one of the desired transgenic/knockout phenotypes, is provided. Such animals are bred together through a series of crosses, backcrosses and selections, to ultimately generate a single animal containing all desired transgene/knockout constructs, where the animal is otherwise congenic (genetically identical) to the wild type except for the presence of the transgene(s) and/or knockout constructs. For example, animals can be generated which comprise both a disrupted AFAP gene and a disruption in one or more genes belonging to an AFAP signaling pathway. In one aspect, animals are generated which comprise a disrupted AFAP gene and which overexpress a gene belonging to an AFAP signaling pathway (e.g., such as an AFAP binding protein and/or AFAP phosphorylating protein). In another aspect, an animal is provided which overexpresses AFAP or which ectopically expresses AFAP and which over- or under-expresses a gene belonging to an AFAP signaling pathway.

In a further aspect, a tissue-specific conditional knockout of AFAP is provided. To provide tissue-specific control of AFAP expression, an AFAP construct is generated which comprises AFAP nucleic acids flanked by recombinase recognition sequences. Recombinase recognition sequences are recognized by recombinases which will excise or invert sequences between them. Placement of such sequences can be used to control the activation or repression of AFAP expression in a tissue-specific manner. For example, recombination can be used to separate AFAP coding sequences from positive regulatory sequences and thus tissue-specific recombinase activity can be used to shut off or decrease AFAP gene expression. Recombination also can be used to bring AFAP coding sequences in proximity to AFAP positive regulatory sequences such that the tissue-specific action of the recombinase will turn on or enhance AFAP gene expression.

Recombinase recognition sequences also can be engineered to flank sequences which interfere with the expression of AFAP. For example, recombinase recognition sequences can be used to flank nucleic acids expressing antisense AFAP molecules or antagonists of AFAP function, such that excision of these sequences results in AFAP expression. By placing such molecules in proximity to positive or negative regulatory sequences, induction of these antisense molecules or antagonists can be controlled further.

In a preferred aspect, the recombinase/recombinase recognition sequence system used is the cre/loxP recombinase system of bacteriophage P1 (Lakso et al., 1992, Proc. Natl. Acad. Sci. USA 89: 6232-6236; Orban et al., 1992, Proc. Natl. Acad. Sci. USA 89: 6861-6865). Cre recombinase catalyzes the site-specific recombination of an intervening target sequence located between loxP sequences. LoxP sequences are 34 base pair nucleotide repeat sequences to which the Cre recombinase binds and are required for Cre recombinase-mediated genetic recombination. The orientation of loxP sequences determines whether the intervening target sequence is excised or inverted when Cre recombinase is present (Abremski et al., 1984, J Biol. Chem. 259: 1509-1514). When loxP sequences are oriented as direct repeats intervening sequences are excised upon binding of Cre. When loxP sequences are oriented as inverted repeats, intervening sequences are inverted. Expression of the recombinase can be regulated by promoter elements which are subject to regulatory control, e.g., tissue-specific promoters as are known in the art. This regulated control will result in genetic recombination of the target sequence only in cells where Cre recombinase expression is mediated by the promoter element. Thus, expression of AFAP polypeptides can be regulated in a tissue-specific manner via control of Cre recombinase expression. The FLP recombinase system of Saccharomyces cerevisiae (O'Gorman et al., 1991, Science 251: 1351-1355; WO 92/15694) can be used in a similar manner to mediate tissue-specific regulation of AFAP polypeptides, fragments, and variants.

Animals conditionally expressing AFAP sequences also can be provided by operably linking prokaryotic promoter sequences to AFAP coding regions. Such promoter sequences require prokaryotic transcriptional activators to be simultaneously expressed in order to express AFAP polypeptides and therefore, AFAP expression can be regulated by operably linking the transcriptional activators to a tissue-specific regulatory element (e.g., a promoter). Exemplary prokaryotic promoters and corresponding transactivating prokaryotic proteins are described in U.S. Pat. No. 4,833,080, the entirety of which is incorporated herein by reference.

In one aspect, the recombinase promoter or the prokaryotic transcriptional activator promoter is one which is active in a cell in which AFAP polypeptides are normally expressed and where AFAP malfunction is associated with disease, e.g., the promoter is active in one or more of neural tissue, lung tissue, breast tissue, colon tissue, prostate tissue and hematopoietic cells tissue.

In addition to tissue-specific regulation of AFAP expression, the invention also contemplates providing developmental stage-specific control of AFAP expression. For example, promoters which are active at particular stages of development can be linked to the recombinases and prokaryotic transactivators described above to selectively turn AFAP expression on or off at particular developmental stages, such as during the development of the CNS or during the maturation of hematopoietic progenitor cells.

In still further aspects, the recombinases and prokaryotic transactivators can be coupled to promoters which are inducible or repressible upon exposure to an agent, such that administering the agent to an animal can be used to drive expression of AFAP polypeptides in one or more tissue types/developmental stages of the animal.

Applications

Diagnostic Assays

AFAP-110 polypeptides are expressed in many cells lines such as fibroblast, epithelial, endothelial, and hematopoietic cell lines (see, Flynn et al., 1995, supra). Expression patterns in cells are limited mainly to stress filaments and the cell membrane, although some peri-nuclear localization has been observed (Flynn et al., 1993, supra). In tissues, AFAP-110 is expressed in brain and muscle tissue (e.g., heart, skeletal muscle, intestinal smooth muscle), as well as in kidney, liver, lung, and breast myoepithelial cells. In brain, AFAP-110 is expressed at high levels in the Purkinje cell layer of the cerebellum and olfactory bulb. AFAP-110 also appears to be limited to primary order neurons that traverse from the nose to the glomeruli where these neurons terminate with second order neural cells. Olfactory neurons of this type are unique in that they must regenerate after 40 days (see, Murray and Calof, 1999, Semin. Cell Dev. Biol. 10: 421-431). The new olfactory neurons mature from stem cells and the nerve termini must migrate and synapse with second order neurons in specific glomeruli.

It is a discovery of the present invention that AFAP-110 is not expressed in a number of cancer cell lines including the breast cancer cell line, MCF-7, the colon cancer cell line, HT29, and the prostate cancer cell line, DU-145. It is also a discovery of the instant invention that certain forms of AFAP-110 can activate Src. For example, deletion mutants of the leucine zipper domain can activate Src and are hyperphosphorylated resulting in the appearance of Src in actin-rich lamellipodia. A point mutation of the SH3 domain in such deletion mutants can prevent Src activation. A deletion in the PH domain also can revert this effect. Because dominant positive RhoA mutations will block the formation of actin filament rosettes (e.g., a cancerous phenotype), transformation of cells by activated Src appears to require a Rho-dependent interaction with an AFAP polypeptide. cSrc is activated in a number of human cancers including breast cancer, colon cancer, prostate cancer, lung cancer (e.g., small lung cell carcinoma), neuroblastoma, Ewing sarcoma and rhabdomyosarcoma (Cartwright et al., 1990, supra; Rosen et al., 1986, supra).

Accordingly, the invention provides a method of screening for enhanced risk of cancer (compared to normal patient populations) or the presence of cancer. In one aspect, the cancer is selected from the group consisting of breast cancer, colon cancer, prostate cancer, lung cancer (e.g., small lung cell carcinoma), a cancer involving neural cells (e.g., such as neuroblastoma), Ewing sarcoma and rhabdomyosarcoma.

In one aspect, the method comprises obtaining a biological sample from a patient (e.g., a tissue, cell(s), bodily fluid), contacting the sample with a molecular probe reactive with an AFAP polypeptide, modified form thereof or variant thereof, and detecting the reactivity of the molecular probe. The molecular probe can be a nucleic acid, antibody, AFAP binding partner, aptamer, and the like. In one aspect, the molecular probe is used to detect a decrease in the expression of an AFAP nucleic acid and/or polypeptide wherein a decrease observed provides a determination of an enhanced risk of cancer or the presence of cancer. Preferably, the decrease is determined with reference to a control biological sample from a normal patient.

In another aspect, the molecular probe is used to detect a decrease in the expression of an AFAP nucleic acid and/or polypeptide wherein an increase observed provides a determination of an enhanced risk of obesity. Preferably, the increase is determined with reference to a control biological sample from a normal patient.

In another aspect, the molecular probe is used to detect a mutant form of an AFAP gene, transcript, and/or polypeptide. In one aspect, a mutation in the leucine zipper domain of an AFAP polypeptide is detected. The mutation can be a deletion, insertion or substitution of one or more amino acids in an AFAP polypeptide.

In another aspect, the probe is specific for a particular modified form of AFAP. In one aspect, the probe specifically recognizes PKC-mediated modifications of an AFAP polypeptide. In another aspect, the probe specifically recognize Src-mediated modifications of an AFAP polypeptide. In still another aspect, hyperphosphorylation of an AFAP polypeptide is detected (e.g., by phosphoamino acid analysis as described in Boyle et al., 1991, Methods Enzymol. 201: 110-49).

In a further aspect, the probe is used to detect an activated form of a polypeptide which down-regulates the expression of AFAP polypeptides. In one aspect, the polypeptide is a constitutively activated or otherwise mutated form of Akt.

AFAP nucleic acids described above can be used in these assays in hybridization-based screening assays such as Southerns (e.g., to detect deleted or other mutated AFAP genes associated with cancer), Northerns, RT-PCR, array-based assays and the like (e.g., to detect decreased expression of AFAP transcripts or expression of aberrant AFAP transcripts). AFAP polypeptides and/or modified forms thereof can be detected using standard immunoassays using the antibodies described above. Immunoassays include, but are not limited to, radioimmunoassays, enzyme immunoassays (e.g. ELISA), immunofluorescence (such as immunohistochemical analyses), immunoprecipitation, latex agglutination, hemagglutination, and histochemical tests. Such assays are routine in the art.

It should be obvious to those of skill in the art that such assays can be used in conjunction with other diagnostic assays and in conjunction with the monitoring of other physiological responses of a patient (e.g., presence of abnormal areas of cell growth and the like).

The invention also contemplates detecting abnormal expression of AFAP polypeptides or modified or variant forms thereof in any pathology associated with an aberrant AFAP signaling pathway. For example, molecular probes according to the invention (e.g., nucleic acids or antibodies) can be used to screen for neurological diseases or respiratory diseases to establish their diagnostic potential to detect such diseases.

In a particularly preferred embodiment, molecular probes according to the invention are used to screen individuals having a neuropsychiatric disorder to identify those individuals with abnormal expression of AFAP. Preferably, such individuals comprise a variant sequence at chromosome 4p16.1 (e.g., a deletion, insertion, and/or substitution of one or more nucleotides). Preferably, the variant sequence is within the AFAP gene. Such probes can be used to identify AFAP nucleic acids and/or polypeptides which are diagnostic of the neuropsychiatric disorder. Individuals having neuropsychiatric disorders initially can be identified according to DSM-IV criteria or can be so identified after detection of a variant AFAP sequence.

In another embodiment, molecular probes are used to screen individuals having a disorder relating to the abnormal proliferation, differentiation, and/or death of hematopoietic cells for variant AFAP sequences, the presence of which can be correlated to increased risk of the disorder. Disorders of hematopoietic cells include, but are not limited to, adult T-cell leukemia; chronic myelocytic leukemia; acute promyelocytic leukemia; thrombocytopenia; hypoplasia; disseminated intravascular coagulation (DIC); myelodysplasia; immune (autoimmune) thrombocytopenic purpura (ITP); and HIV induced ITP.

In general, the invention contemplates methods of screening for disorders associated with variant sequences at chromosome 4p16.1. Such sequences can include variant AFAP sequences or can include sequences which are closely linked to AFAP sequences, such that detection of a particular AFAP allele will be diagnostic for the presence of the variant sequence.

Methods of Using Transgenic Animals and Knockout Animals

The invention also provides methods for studying physiological responses to abnormal AFAP mediated signaling events. As discussed above, cells, tissues, and non-human animals lacking or partially lacking AFAP expression may be developed using recombinant expression vectors of the invention having specific deletion or insertion mutations in the AFAP-110 gene. For example, in one aspect, one or more AFAP domains is deleted or mutated. A recombinant expression vector may be used to inactivate or alter an endogenous gene by homologous recombination to create an AFAP-deficient cell, tissue or animal. In one aspect, the cell, tissue, or animal is deficient for AFAP-110. In another aspect, the cell, tissue, or animal is deficient for AFAP-120. Such animals can be used to monitor the enhanced development of cancer in such animals thereby providing model systems in which to test anti-cancer agents (e.g., drugs, therapeutic antibodies and the like). Such animals can also be used to monitor the development of obesity in such animals thereby providing model systems in which to test anti-obesity agents.

In a particularly preferred aspect, a transgenic animal is provided comprising AFAP regulatory sequence(s) operably linked to a reporter molecule such that expression of the reporter molecule can be used to identify cells in which AFAP signaling pathways are operating. Preferably, such regulatory sequences are able to drive AFAP expression in neuronal cells in which AFAP polypeptides are normally expressed. AFAP regulatory sequences can be identified by selecting AFAP genomic sequences as described above using probes according to the invention or by screening an electronic sequence database of genomic sequences for sequences upstream of AFAP coding sequences to identify a candidate regulatory sequence region. Once a candidate sequence region is identified, regulatory elements within the candidate regulatory sequence region can be determined by routine methods such as deletion analysis, mutagenesis, footprinting, gel shifts and transfection analyses (see, e.g., Sambrook et al., supra).

In a particularly preferred aspect, an AFAP regulatory sequence is operably linked to a GFP molecule such that GFP is expressed in cells which normally express AFAP polypeptides. Because AFAP is expressed in developing neurons, GFP expression may be used to identify regenerating neurons in the CNS. Thus, in one aspect, transgenic animals expressing such reporter constructs can be used to evaluate agents for their effect on neuronal cell regeneration. For example, such animals can be used to determine the efficacy of drugs which might be used to treat neurodegenerative diseases, such as Alzheimer's disease, to identify lead drugs for further testing which maximize neuronal cell regeneration and minimize neuronal cell death.

Additional animal models useful according to the invention include an animal model for obesity. The genetically obese mouse represents an animal model for obesity (Murphy et al., 1997, Proc. Natl. Acad. Sci USA, 94: 13921-13926). The ob/ob mouse is genetically deficient in leptin and exhibits a phenotype that includes obesity and non-insulin-dependent diabetes mellitus. This phenotype closely resembles the morbid obesity seen in humans. In ob/ob mice, a mutation in the ob gene leads to a marked increase in food consumption that results in an increase in adipose tissue mass and a syndrome that resembles morbid obesity in humans. Abnormalities include hypothermia, lethargy, hyperglycemia, glucose intolerance, and hyperinsulinemia resembling non-insulin-dependent diabetes melitus in humans (Murphy et al., supra).

Screening Assays for Therapeutic Agents

The invention still further provides a method for identifying an agent which is capable of binding to an AFAP polypeptide, fragment thereof, modified form thereof, or variant thereof, comprising reacting a AFAP polypeptide, a fragment thereof comprising one or more AFAP domains, a modified form thereof, or variant form thereof, with at least one agent and detecting changes in one or more AFAP activities as described above when compared to the activity of the AFAP polypeptide, fragment thereof comprising one or more AFAP domains, modified form thereof, or variant thereof, which has not been contacted with the agent. In one aspect, the agent is an antibody or antigen-binding fragment thereof.

In another aspect, the assay is performed under conditions which permit the formation of complexes between the agent and the AFAP polypeptide, fragment thereof comprising one or more AFAP domains, or modified form thereof, and agents are selected which modulate the formation of such complexes, by measuring the presence, and preferably, the amount of one or more of: unbound agent, complexes, and unbound AFAP polypeptide, fragment thereof comprising one or more AFAP domains, or modified form thereof. Such molecules can be isolated by conventional isolation techniques, for example, salting out, chromatography, electrophoresis, gel filtration, fractionation, absorption, polyacrylamide gel electrophoresis, agglutination, or combinations of these techniques. Antibodies described above can be used to detect these molecules. One or more of the molecules may be labeled with a detectable label as described above.

Agents which affect an AFAP polypeptide, fragments, modified forms thereof, or variant forms thereof, also may be identified by comparing the pattern and level of expression of the AFAP polypeptide in tissues and cells, in the presence and in the absence of the agent. For example, the effects of the agent on the host cells or transgenic animals of the invention can be monitored. In one aspect, the effect of the agent on one or more AFAP activities in the host cells or cell(s) of the transgenic animals is evaluated. In a preferred aspect, the activity monitored is the ability of the cell(s) to produce a cancer phenotype.

In a further aspect, the activity monitored is increased or decreased angiogenesis. In a further aspect, the activity monitored is an increase in the mass of an adipocyte.

The invention also provides a method for the presence of an agonist or antagonist of the interaction of an AFAP polypeptide, fragment thereof comprising one or more AFAP domains, modified form thereof, or variant thereof, and a substance which binds to any of the foregoing. In one aspect, the method comprises providing a known concentration of an AFAP polypeptide, fragment thereof comprising one or more AFAP domains, modified form thereof, or variant thereof, providing a substance which is capable of specifically binding to the AFAP polypeptide, fragment thereof comprising one or more AFAP domains, modified form thereof, or variant thereof, under conditions which permit the formation of complexes between the substance, providing an agent, and assaying for one or more of complexes, free substance, and non-complexed AFAP polypeptide, fragment thereof comprising one or more AFAP domains, modified form thereof, or variant thereof. Additionally, or alternatively, one or more AFAP activities can be monitored as described above. Preferably, measuring of binding and/or of AFAP activit(ies) is done with reference to a control in the absence of the agent and/or the substance.

The invention also makes it possible to screen for antagonists that inhibit the effects of an agonist of the interaction of AFAP polypeptide with a substance which is capable of binding to an AFAP polypeptide or modified form thereof or variant thereof Thus, the invention also may be used to assay for a substance that competes for the same binding site of an AFAP polypeptide, fragment thereof, or modified form thereof, or variant thereof.

In one aspect, the substance is a polypeptide (e.g., Src, actin, PKCα, or other AFAP binding polypeptide). In another aspect, the agonist or antagonist is synthetic (e.g., a drug). Agonists and antagonists can be identified from purified or partially purified cellular biomolecules, from synthetic compounds, from combinatorial libraries, from phage display libraries and the like. Combinatorial libraries and phage display libraries are commercially available. In one aspect, agents are synthesized based on in silico modeling of the effects of such agents on AFAP interactions with AFAP binding proteins (e.g., such as Src, a PKC polypeptide, and Rho).

It should be obvious to those of ordinary skill in the art that as new interactions are identified between AFAP polypeptides and cellular biomolecules, agonists and antagonists of these interactions can be identified using the methods described above.

As discussed above, AFAP-110 has the ability to activate Src family kinases (in general, polypeptides comprising an SH3 domain) and certain modified forms of AFAP (e.g., comprising mutations in the leucine zipper domain) can constitutively activate Src. Because activation of Src can result in a cancer phenotype, agents which modulate the interactions of AFAP polypeptides and the SH3 domain of Src can be identified as leads to test in the treatment of cancer. Accordingly, in one aspect, an AFAP SH3 binding domain containing polypeptide is provided along with a Src SH3 domain containing polypeptide (either simultaneously or sequentially) and the phosphorylation of the Src polypeptide is monitored (e.g., using an antibody which specifically recognizes the modified form of Src) as a means of monitoring Src activation. In another aspect, activation of Src is monitored by observing one or more downstream molecules in a Src pathway.

As discussed above, AFAP-110 is a substrate for a PKC polypeptide and when activated by phosphorylation of a PH domain, AFAP-110 crosslinks actin filaments, becoming an actin filament bundling protein. Because AFAP-110 is activated by PKCα in motility structures such as filopodia or lamellopodia such as occurs in cancerous cells, in a preferred aspect of the invention, agents are screened for which affect binding of a polypeptide comprising one or more PH domains to PKCα. Preferably, binding of a PKC polypeptide to the PH1 domain (e.g., amino acids corresponding to amino acids 150-250 of the human AFAP polypeptide) of an AFAP polypeptide is monitored. Binding can be assayed for directly (e.g., measuring complexes formed) or can be assayed for indirectly (e.g., by measuring PKCα-mediated phosphorylation of AFAP or activation of Src and/or by measuring bundling of actin filaments and/or the appearance of cell motility structures). As above, agents can be identified from purified or partially purified cellular biomolecules, from synthetic compounds, from combinatorial libraries, from phage display libraries and the like, and can be identified initially by in silico modeling.

The agents, agonists, and antagonists identified using the methods of the invention may be used in the treatment of conditions involving the perturbation of signaling pathways involving AFAP polypeptides and modified forms thereof. In one aspect, the condition is cancer (e.g., such as breast cancer, colon cancer, prostate cancer, lung cancer, neuroblastoma, Ewing sarcoma and rhabdomyosarcoma). In another aspect, the condition is a neurological disease (which can include a cancer involving neural cells, such as neuroblastoma or a neurodegenerative or neuropsychiatric disease). In still a further aspect, the condition is a respiratory disease or other condition which can be affected by aberrant cell motility. In still a further aspect, the condition is obesity. In still a further aspect, the condition is any condition that is affected by angiogenesis or aberrant angiogenesis (for example, increased or decreased angiogenesis).

The agents, agonists, and antagonists may be formulated into compositions for administration to individuals suffering from any of these conditions. Therefore, the present invention also relates to a composition comprising the agent, agonist, or antagonist and a pharmaceutically acceptable carrier. Suitable pharmaceutically acceptable carriers are described, in Remington 's Pharmaceutical Sciences, Mack Publishing Company, Easton, Pa., USA 1985, for example, the entirety of which is incorporated by reference herein. The activity of compositions of the invention may be confirmed in suitable animal experimental model systems as are known in the art or which are generated as described above.

A method for treating or preventing a condition involving an AFAP regulatory system also is provided comprising administering to a patient in need thereof, a composition of the invention. The compositions according to the invention can be administered in a therapeutically effective amount to a patient such as a human being having a pathology associated with an aberrant AFAP signaling pathway. In one aspect, a therapeutically effective amount is an amount effective, at dosages and for periods of time necessary to achieve regression of a cancer phenotype as measured by any of: decrease in tumor size, reduction in numbers of abnormally proliferating cells in a biological sample (such as a biopsy sample, blood sample, serum sample, lymph sample, urine sample, and the like), reduction in the amount of a cancer-specific marker in an biological sample (e.g., PSA, HER2/neu, etc.), and the like. In one aspect, a therapeutically effective amount is an amount effective to reduce a cancer phenotype by at least about 10%, about 20%, about 30%, about 40%, about 50%, about 70%, about 80%, about 90%, and preferably at least about 100% (e.g., at least about 2-fold, at least about 4-fold, at least about 10-fold, at least about 20-fold, and preferably, at least about 50-fold.

In another aspect, a therapeutically effective amount is an amount effective, at doasges and for periods of time necessary to achieve reversal of an obese phenotype as measured by a decrease in body mass.

In another aspect, a therapeutically effective amount is an amount effective at dosages and for periods of time necessary to achieve either activation or repression of angiogenesis.

A therapeutically active amount of a composition may vary according to factors such as the disease state, age, sex, and weight of the patient, and dosage may be adjusted using methods routine in the art to provide the optimum therapeutic response. For example, several divided doses may be administered daily or the dose may be proportionally reduced or increased as indicated by monitoring one or more therapeutic endpoints.

The composition may be administered in a convenient manner such as by injection (subcutaneous, intravenous, etc.), oral administration, by inhalation, transdermal application, by vaginal or rectal administration. Depending on the route of administration, the active substance may be coated in a material to protect the compound from the action of enzymes, acids and other natural conditions which may inactivate the compound.

Variations, modification, and other implementations of what is described herein will occur to those of skill in the art without departing from the spirit and scope of the invention and the following claims. References, patents and patent publications cited herein are herein incorporated by reference in their entirety. The invention is further described in following Examples.

### EXAMPLES

### Example 1

### Expression of Active PKC Results in the Activation of cSrc and Cellular Tyrosine Phosphorylation, in vivo

AFAP-110 has an intrinsic capability to alter actin filament integrity that can be regulated by its leucine zipper motif. Deletion of the leucine zipper motif, AFAP-110^{Δlzip}, will affect changes in the actin-based cytoskeleton, which is typified by the deposition of actin into rosette-like structures and the induction of motility structures (Qian et al., 1998, supra, Qian et al., 2000, supra) and concomitant with an ability to activate cSrc in an SH3-dependent fashion, discussed herein below.

Immunofluorescence

For cells transfected with Flag-tagged PKCα, rhodamine phalloidin (1 :1000) and anti-Flag ab (1:1000) were used. Anti-mouse Alexa 488 (1:200) was used to visualize the anti-Flag ab. Anti-phosphotyrosine (1:100) and antiSrc(pY416) were used in separate experiments to label tyrosine phosphorylated proteins and active Src family kinases, respectively. These polyclonal antibodies were visualized with anti-rabbit Alexa 633. For cells co-transfected with Flag-tagged PKCα and GFP-tagged forms of AFAP-110, anti-Flag, anti-ptyr and antiSrc(pY416) were used as above. In this instance, anti-mouse TRITC was used to visualize anti-Flag antibody, while phospho-specific antibodies were labeled as above. Cells were washed and mounted on slides with Fluoromount (Fisher). A Zeiss LSM 510 microscope was used to gather images. Scale bars were generated and inserted by LSM 510 software. For all figures, representative cells are shown (>100 cells examined per image shown).

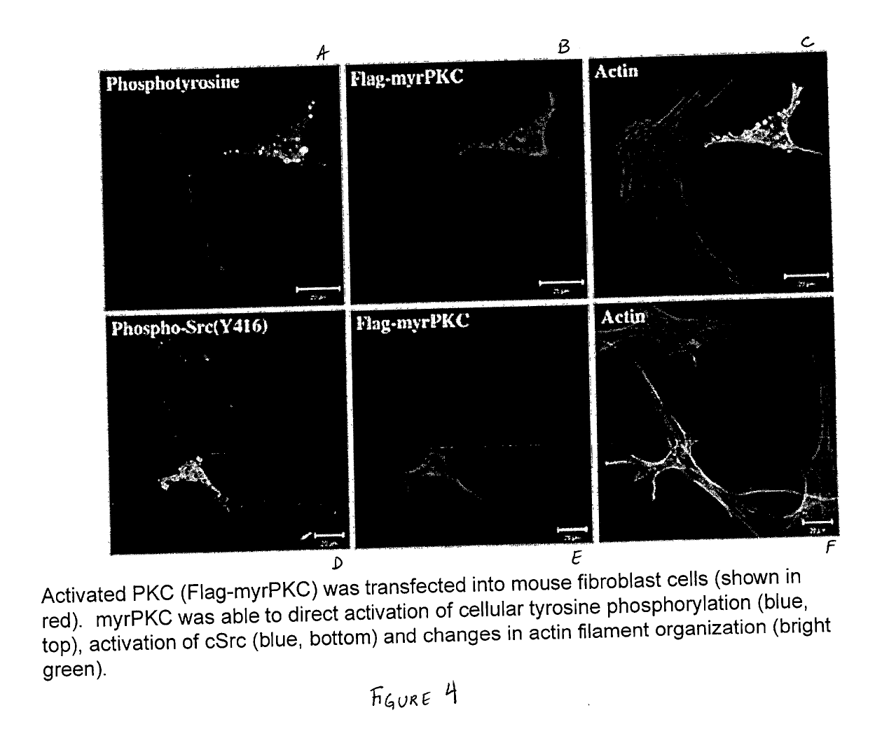

C3H10T1/2 cells were transfected with Flag-tagged myristoylated PKCα (FIG. 4, panels B and E), a constitutively active form of the enzyme. This form of PKC has been shown to maintain constitutive kinase activity by virtue of its membrane association.

Expression of active PKC results in tyrosine hyperphosphorylation and Src family kinases. C3H10T1/2 cells expressing Flag-myrPKCα were fixed and labeled as described to visualize Flag-myrPKCα, shown here as red (FIG. 4, Panels B and E), actin, shown as green (FIG. 4, Panels C and F). These cells were also labeled with either anti-phosphotyrosine (FIG. 4, Panel A) or anti-SrcP(Y416) (FIG. 4, Panel D), both shown as blue.

Labeling these cells with anti-phosphotyrosine antibodies (Transduction Laboratories) demonstrated increased levels of phosphotyrosine containing proteins (FIG. 4, panel A). Thus, cellular proteins are hyperphosphorylated on tyrosine residues in response to PKC activation. This hyperphosphorylation was seen only in cells expressing Flag-myrPKC, which also displayed a reorganization of the actin cytoskeleton, compared to non-transfected cells (FIG. 4, panel C). FIG. 4, panel D shows the results seen upon labeling similar cells with anti-phospho-Src (Y⁴¹⁶), which indicated an increase in Src family kinase activity in PKCα expression cells, compared to non-transfected surrounding cells. Again, these cells displayed a reorganization of the cytoskeleton (FIG. 4, panel F). Expression of wild type or kinase-dead PKC had no effect on actin filament integrity or cellular phosphotyrosine levels (data not shown). These results demonstrate that cellular tyrosine kinases, such as cSrc, can be activated in response to PKCα activation.

### Example 2

### PKC Activation of cSrc is Regulated by AFAP-110

GFP-tagged forms of AFAP-110 were employed to examine the potential requirement for AFAP-110 in mediating PKCα-directed cytoskeletal rearrangements. AFAP-110 mutants deficient for interactions with the Src SH3 domain (AFAP-110^{71A}) or that contained deletions in the PH domain that are predicted to abrogate binding to PKCα (AFAP-110^{Δ180-226}) were used to determine if PKCα could activate cSrc in an AFAP-110 dependent fashion. The AFAP-110^{71A} mutant has been shown to be unable to bind to the Src SH3 domain or form a stable complex with Src^{527F} (Guappone and Flynn, 1997, supra) and within the context of AFAP-110^{71A/Δlzip}, prevents activation of cSrc, cellular tyrosine phosphorylation or actin filament changes (discussed herein below). The AFAP-110^{Δ180-226} mutant is predicted to be unable to forge a PH domain mediated interaction with PKCα, based on the results discussed herein.

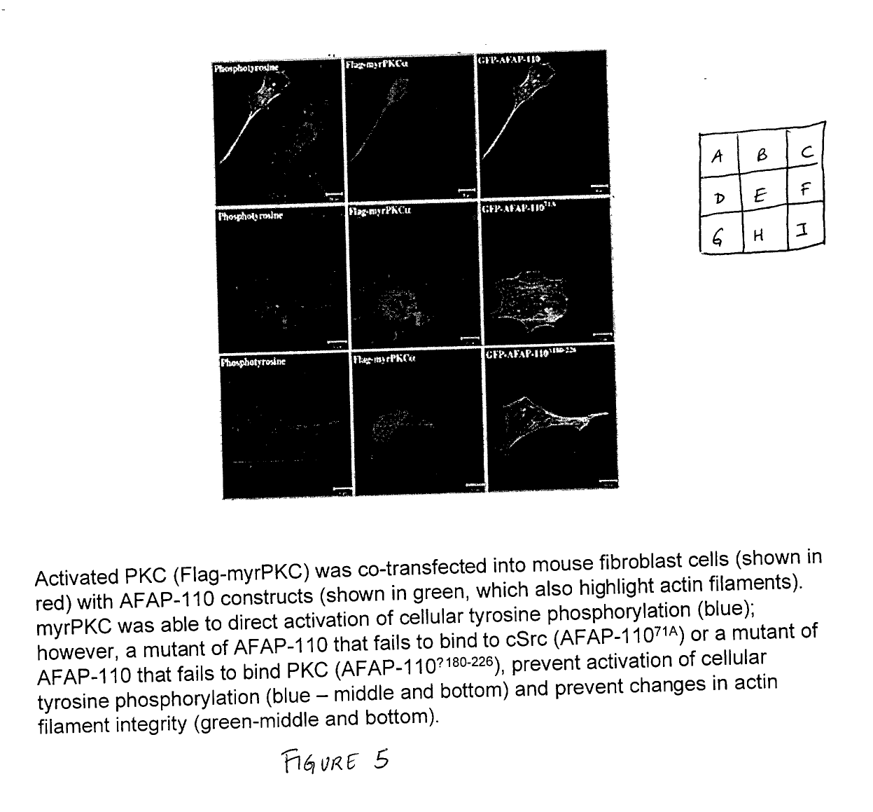

FIG. 5 demonstrates that the activation of tyrosine hyperphosphorylation by PKC requires the integrity of both the SH3 binding motif and PH domain of AFAP-110. C3H10T1/2 cells co-expressing GFP-AFAP-110 (Panel C) and myristoylated, Flag-tagged PKC were labeled with anti-Flag (Panel B) and anti-phosphotyrosine (Panel A). These cells display increased immunoreactivity with the phospho-specific antibody compared to non-transfected surrounding cells. Cells co-expressing GFP-AFAP-110^{71A} (Panel F) and myristoylated, Flag-tagged PKC (Panel E) were similarly labeled to visualize anti-phosphotyrosine (Panel D). These cells display immunoreactivity with the phospho-specific antibodies equivalent to non-transfected cells as well as numerous actin filaments and levels of lamellipodia equivalent to non-transfected cells. Identical results were seen upon co-expression of GFP-AFAP-110^{Δ180-226} (Panel I) with Flag-myrPKCa (Panel H). These cells were also labeled with anti-phosphotyrosine (Panel G). Co-expression of GFP-AFAP-110 and Flag-myrPKCα resulted in the hyperphosphorylation of cellular proteins on tyrosine, as seen in FIG. 5, panel A. Cytoskeletal rearrangements were noted upon examination of GFP-AFAP-110 (Panels C) in these cells, which consistently co-localizes with actin structures (Qian et al., 2000, supra). GFP-AFAP-110 itself was unable to induce changes in actin filament integrity (Qian et al., 2000, supra).

GFP-AFAP-110^{71A} (panel F) co-expressed with Flag-myrPKCα (panel E) blocked the increased tyrosine phosphorylation of cellular proteins (panel D). This mutant also inhibited the reorganization of the actin cytoskeleton in these cells, as shown in panel F. GFP-AFAP-110^{Δ180-226} (panel I) co-expressed with flag-myrPKCα (panel H) also prevented increased tyrosine phosphorylation of cellular proteins (panel G) as well as cytoskeletal rearrangements. Phospho-specific Src antibodies were used to label cells as above to determine the role of AFAP-110 in the PKC-mediated activation of Src.

FIG. 6 demonstrates that the activation of Src family kinases by PKC requires the integrity of both the SH3 binding motif and PH domain of AFAP-110. C3H10T1/2 cells co-expressing GFP-AFAP-110 (Panel C) and myristoylated, Flag-tagged PKC were labeled with anti-Flag (Panel B) and anti-SrcP(Y416) (Panel A). These cells displayed increased immunoreactivity with the phospho-specific antibody compared to non-transfected surrounding cells. Cells co-expressing GFP-AFAP-110^{71A} (Panel F) and myristoylated, Flag-tagged PKC (Panel E) were similarly labeled to visualize anti-SrcP(Y416) (Panel D). These cells displayed immunoreactivity with the phospho-specific antibodies equivalent to non-transfected cells as well as numerous actin filaments and levels of lamellipodia equivalent to non-transfected cells. Identical results were seen upon co-expression of GFP-AFAP-110^{Δ180-226} (Panel I) with Flag-myrPKCa (Panel H). These cells were also labeled with anti-SrcP(Y416) (Panel G).

FIG. 6 indicates that the co-expression of GFP-AFAP-110 (panel A) with Flag-myrPKCα (panel B) results in both the increased labeling of cells with phospho-Src(Y416) (panel A) and the reorganization of the actin cytoskeleton, as shown with GFP-AFAP-110. Expression of either AFAP-110^{71A} (panel F) or AFAP-110^{Δ180-226} (panel I) with Flag-myrPKCα (panels E and H) blocked the increased labeling of transfected cells with phospho-Src(Y416) (panels D and G). These mutants also inhibit the reorganization of the actin cytoskeleton. Therefore, the integrity of both the SH3 motif and amino-terminal PH domain of AFAP-110 are required for the activation of Src by PKC.

### Example 3

### rAFAP-110 Cooperatively Binds to Actin Filaments Through a Lateral Association

Previous data demonstrated that a polypeptide encoding the carboxy terminal actin binding domain in AFAP-110 was sufficient to direct binding to actin filaments, in vivo and in vitro (Qian et al., 2000, supra).

Plasmid Constructs

AFAP-110, AFAP-110^{Δcterm}, and AFAP-110^{Δlzip} DNA fragments were cloned into pGEX-6p-1 vector to construct pGEX-6p-1-AFAP-110, pGEX6p-1-AFAP-110^{Δcterm}, and pGEX-6p-1-AFAP-110^{Δlzip} vectors, respectively, from pGEX-2T-AFAP-110 series cutting with MluI and BamHI restriction enzymes. The pEGFP-c3 Expression system from Amersham Pharmacia (Piscataway, N.J.) was used to express GFP-tagged forms of AFAP-110. AFAP-110 was cloned into this vector as previously described (Qian et al., 2000, supra). CMV-AFAP-110^{Δ180-226} was previously described (Baisden et al., 2001, supra). Fragments from CMV-AFAP-110 and CMV-AFAP-110^{Δ180-226} were subcloned into pEGFP-c3-AFAP-110 to create full-length, GFP-tagged forms of these mutants. Flag-myr-PKC was a kind gift from Alex Toker.

Protein Purification

Recombinant AFAP-110, recombinant AFAP-110^{Δlzip}, and recombinant AFAP-110^{Δcterm} were purified after production as a GST bacterial fusion protein, using the PreScission Protease system (Amersham Pharmacia) as previously described (Qian et al., 2000, supra).

Actin Binding Assay

G-actin was polymerized to F-actin at 5 mM Tris-HCl, pH 8.0, 0.2 mM CaCl₂, 0.5 mM DTT, 5 mM MgCl₂, and 2 mM ATP at room temperature (RT) for 1 h. Different concentrations of rAFAP-110 were incubated with 2 μM F-actin in 5 mM MgCl₂, 1 mM EGTA, 2 mM ATP, 50 mM KCl at RT for 30 min. The reactions were centrifuged at 150,000×g for 1 h at 4° C., and then both pellets and supernatants were collected and analyzed by SDS-PAGE gel.

Analysis of Actin Binding

Binding constants were derived from the sedimentation of rAFAP-110 by actin. Accordingly, rAFAP-110 in the pellet and supernatant correspond to the bound and free rAFAP-110, respectively. The sum of densities of rAFAP-110 bands in the supernatant and pellet corresponds to the total mass of rAFAP-110 in the sample. The data representing the bound rAFAP-110 as a function of free rAFAP-110 were fit by nonlinear least squares to both the Langmuir (B=B_{MAX}/(1+K_{d}/F)) and Hill assays (B=B_{MAX}/(1+K_{d}/F)ⁿ) were performed with 2 μM F-actin filaments and different concentrations of rAFAP-110 in 5 mM MgCl₂, 1 mM EGTA, 2 mM ATP, and 50 mM KCl at 20,800×g for 30 min at 4° C. After the centrifugation, both supernatant and pellets were analyzed by SDS-PAGE gel. Confocal microscopy assays were performed as described (Ishikawa et al., 1998, J. Biol. Chem., 273:26991-26997). Briefly, 2 μM F-actin containing 10% rhodamine-phalloidin-labeled F-actin were mixed with different concentrations of rAFAP-110 or other purified recombinant proteins in 5 mM MgCl₂, 1 mM EGTA, 2 mM ATP, and 50 mM KCl at RT for 30 min. After the incubation, the reactions were applied between glass slides and coverslips and observed under confocal microscopy (Zeiss, Oberkochen, Germany). Samples for negative staining were adsorbed to grids coated with nitrocellulose and stabilized with carbon (Ernest F. Fullam, Latham, N.Y.). Unbound protein was removed by successive washes with buffer and water before staining with 1% uranylacetate (Cooper and Pollard, 1982, Methods Enzymol., 85(PtB), 182-210; Pollard and Cooper, 1982, Methods Enzymol., 85(PtB), 211-233).

FIG. 7A demonstrates that both rAFAP-110 and rAFAP-110^{Δlzip} bind to actin filaments directly. Either rAFAP-110 or rAFAP-110^{Δlzip} were incubated with actin filaments at 30° C. for 30 min and were centrifuged at 150,000×g for 1 h. Both supernatant (S) and pellet (P) were applied to 10% SDS-PAGE gel, followed by Coomassie blue stain.

Purified rAFAP-110 was shown to copellet with F-actin by high-speed copelleting assays, demonstrating a direct association, and deletion of the leucine zipper motif (AFAP-110^{Δlzip}) did not abolish this direction association (150,000×g; FIG. 7A). FIG. 7B demonstrates EM negative staining. The purified rAFAP-110 was incubated with actin filaments. The negative staining image was taken at 16,000 magnification. Black arrows indicate rAFAP-110 aggregates; and white arrow indicates actin filaments. An examination of negative stained actin filaments preincubated with rAFAP-110 revealed that F-actin was decorated with ellipsoid structures having a long axis of 15-30 nm (FIG. 7B). These large structures likely represent multimers of rAFAP-110, because F-actin alone revealed no such structures, and this system included only purified rAFAP-110 and purified G-actin that had been polymerized to F-actin. The electron micrographs indicate that rAFAP-110 binds to the sides of actin filaments, which was supported by the fact that one mole of actin in filaments bound up to 1.7 mol of rAFAP-110 (FIG. 7C). FIG. 7C is a graph of bound rAFAP-110 versus free rAFAP-110. rAFAP-110 at 0.18,0.29,0.70, 1.34,2.63,3.73,4.67, 5.11,5.34,5.48, and 5.66 μM was incubated with 2 μM concentration of G-actin polymerized into actin filaments and were then centrifuged at 150,000×g. Both supernatants (S) and pellets (P) were applied to SDS-PAGE gel, followed by Coomassie staining. Density of AFAP-110 determined by scanning densitometry was fit as described.

Interestingly, some actin filaments in the field with rAFAP-110 were occasionally naked and had no rAFAP-110 aggregates decorating them (white arrows, FIG. 7B). These results suggested that the binding of rAFAP-110 to actin filaments could be cooperative. Evidence for cooperativity in binding was explored using high-speed copelleting data. The distinctive S-shaped appearance of the binding data when plotted on a linear scale indicated positive cooperativity (FIG. 7C), and the data did not fit (least-squares fit) a noncooperative binding isotherm. However, the data were well described with a least-squares fit to the Hill equation, a cooperative model of binding. The best fit parameters K_{d}, B_{MAX}, and Hill coefficient, n, were 0.29 μM, 4.2 μM, and 3.2, respectively (FIG. 7C). Results of a second experiment were 0.24 μM, 1.5 μM, and 2.6 for K_{d}, B_{MAX}, and n, respectively. A Hill coefficient greater than one confirms cooperative binding. The B_{MAX} was about twofold greater than the 2 μM actin monomer concentration in the assay. The K_{d} is in the range of concentration of AFAP-110 in the cell. These data indicate that AFAP-110 can bind actin filaments directly and that binding may serve to recruit additional AFAP-110 molecules to bind actin filaments.

### Example 4

### AFAP-110 Requires its Carboxy Terminus to Cross-Link Actin Filaments

Actin filaments are unable to efficiently pellet at 20,800×g unless cross-linked to form heavier particles (either isotropic networked or bundled actin filaments; Cooper and Pollard, 1982, supra; Pollard and Cooper, 1982, supra; Meyer et al., 1990, J. Cell. Biol. 110:2013-2024; Wachsstock et al., 1993, Biophys. J., 65: 205-214; Rybakova et al., 1996, J. Cell. Biol., 135:661-672). FIG. 8A demonstrates the results of a low-speed cosedimentation assay. The purified recombinant proteins were incubated with actin filaments and then were centrifuged at 20,800×g. Both supernatants (S) and pellets (P) were applied to SDS-PAGE gel, followed by Coomassie staining. 1, rAFAP-110 with actin filaments; 2, rAFAP-110^{Δcterm} with actin filaments; 3, rAFAP-110 only; 4, rAFAP-110^{Δcterm} only; 5, actin filaments only. FIG. 8B is a graph of cross-linked actin filaments versus free rAFAP-110. rAFAP-110 at 0.05,0.11,0.21,0.43, 0.86, and 2.14 μM was incubated with the 2 μM concentration of G-actin polymerized into actin filaments. After the incubation, the reactions were centrifuged at 20,800×g. Both supernatants and pellets were applied to SDS-PAGE gel, followed by the Western blot analysis to detect rAFAP-110 and Coomassie blue staining to detect actin, respectively. The data of cross-linked actin filaments and free rAFA-110 were gathered by scanning densitometry of SDS-PAGE gel analysis. The least-squares fit of the data to the Hill equation gave 0.26 μM for K_{d}, 1.63 μM for B_{MAX}, and 1.7 for n.

Actin filaments efficiently sedimented when preincubated with rAFAP-110, whereas constructs of rAFAP-110 lacking the actin binding domain, e.g., rAFAP-110^{Δcterm} or with no addition of rAFAP-110 proteins, were unable to efficiently pellet F-actin (FIG. 8A), confirming that rAFAP-110 can cross-link actin filaments. Actin filament cross-linking, as determined by the amount of actin that sedimented at low speed, was dependent on the free concentration of rAFAP-110. At saturation, 81.5% of the actin (1.63 μM/2.0 μM total) was cross-linked and sedimented. Half maximal cross-linking occurred with 0.26 μM rAFAP-110, which is close to the predicted K_{d} for the association of rAFAP-110 with actin filaments, 0.29 μM AFAP-110 (FIG. 8B).

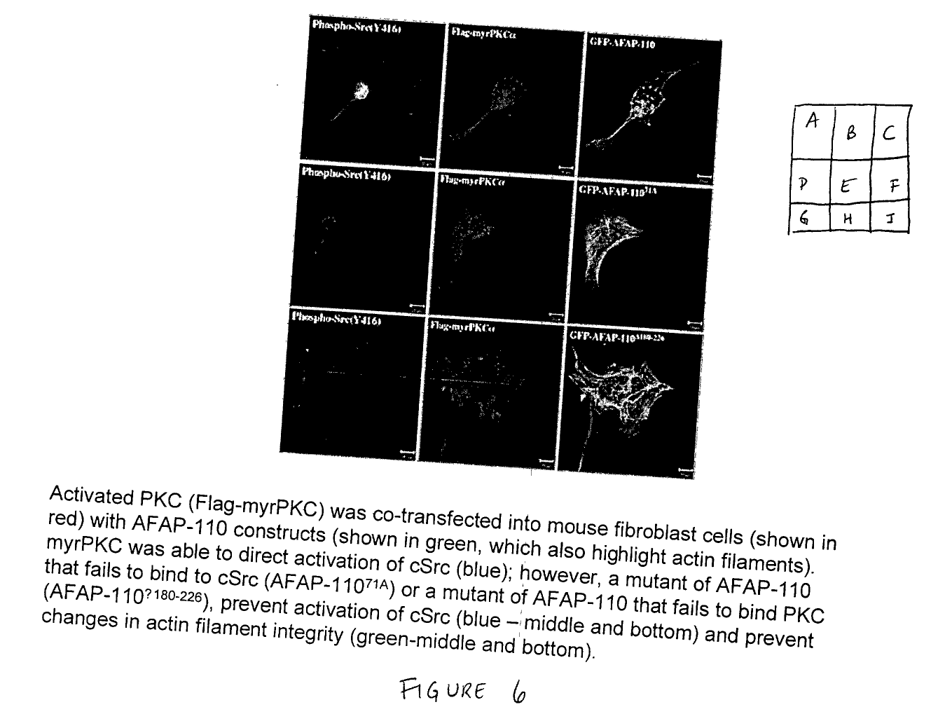

To further test AFAP-110's capability to cross-link actin filaments, we analyzed rhodamine-phalloidin-labeled actin filaments for cross-linking by confocal microscopy. In FIG. 9A, purified rAFAP-110 or rAFAP-110^{Δcterm} were incubated with rhodamine-phalloidin-labeled actin filaments. After the incubation, the reactions were observed with a Zeiss confocal microscope. 1, actin filaments only; 2, rAFAP-110 with actin filaments; 3, rAFAP-110^{Δcterm} with actin filaments. FIG. 9B presents confocal microscopy images. Different concentrations of α-actinin (top two panels, low=0.0625 μM and high=1.25 μM) and rAFAP-110 (bottom two panels, low=0.106 μM and high=2.1 μM) were incubated with rhodamine-phalloidin-labeled actin filaments. After the incubation, the reactions were observed with a Zeiss confocal microscope. Actin filaments labeled with rhodamine-phalloidin appeared uniformly fluorescent in the absence of an actin binding protein (FIG. 9A1). In the presence of 1.3 μM rAFAP-110, actin filaments were organized into a lacy pattern of swollen and interconnected fluorescent tubes (FIG. 9A2). Because the varicose pattern was absent when the actin binding-deficient construct rAFAP-110^{Δcterm} were added (FIG. 9A3), the confocal fluorescence pattern likely results from actin filament cross-linking. Electron microscopy was used to confirm rAFAP-110's ability to cross-link actin filaments.

### Example 5

### Comparison of Cross-Linking by α-Actinin and rAFAP-110

There are two major types of cross-linked actin filament structures: networks and bundles. The networked actin filaments would be predicted to be analogous to a meshwork gel without changing the isotropic nature of actin filaments, while bundled actin filaments are predicted to pack in an anisotropic manner (Wachsstock et al., 1993, Biophys. J., 65:205-214). These data demonstrate how different concentrations of rAFAP-110 affects its ability to cross-link actin filaments. Two different concentrations of α-actinin were used as controls, because α-actinin has the ability to cross-link actin filaments in a dose-dependent manner, networking actin filaments at low concentrations and bundling actin filaments at high concentrations (Meyer and Aebi, 1990, J. Cell Biol., 110:2013-2024; Wachsstock et al., 1993, supra). FIG. 9B demonstrates that low concentrations of α-actinin (0.0625 μM) did not change the organization of actin filaments significantly, whereas high concentration of α-actinin (1.25 μM) caused actin filaments to aggregate (FIG. 9, B1 and B2). These data indicate that immunofluorescence confocal microscopy is a reliable technique to analyze changes in actin filament cross-linking and organization. To analyze the effects of AFAP-110 on actin filaments in vitro, analogous concentrations of rAFAP-110 were used and the effects examined by confocal microscope immunofluorescence. The data demonstrate that low concentrations of rAFAP-110 (0.105 μM) did not change the organization of actin filaments, whereas the high concentrations of rAFAP-110 (2.1 μM) caused some aggregation of actin filaments into large branched structures (FIGS. 9B3 and B4). At low concentration, rAFAP-110 may cross-link actin filaments into isotropic network structures, whereas at high concentrations, rAFAP-110 may cross-link actin filaments into anti-isotropic bundle structures. EM results confirm that rAFAP-110 cross-links actin filaments in a dose-dependent manner. The morphology of actin filaments were unchanged when they were incubated with a low concentration of rAFAP-110, whereas high concentration of rAFAP-110 changed the morphology of actin filaments, as evidenced by an increase in the number of cross-linked actin filaments into fiber structures when examined by electron microscopy.

### Example 6

### AFAP-110 is Both a Binding Partner and Substrate of PKCα

Cellular signals directed by Src^{527F} alter the conformation of AFAP-110 and are hypothesized to reduce its capacity to multimerize (Qian et al., 1998, supra), which could affect the ability of AFAP-110 to cross-link actin filaments. Previous data predicted that tyrosine phosphorylation may not be responsible for the change in AFAP-110 conformation in response to Src^{527F} (Qian et al., 1998, supra). AFAP-110 is a predicted substrate for PKC phosphorylation (Flynn et al., 1993, supra; Baisden et al., 2001 a, supra), PKC is activated in response to Src^{527F} (Spangler et al., 1989, Proc. Natl. Acad. Sci. USA, 86:7017-7021) and PKC activation directs changes in actin filament integrity (Kiley et al., 1992, supra). Purified recombinant AFAP-110 was incubated with recombinant PKCα at a molar ratio of 20:1 substrate to enzyme in the presence of radiolabeled ATP.

In vitro Kinase Assay

PKC kinase assays were carried out according to Current Protocols in Molecular Biology (Carter, 1997). Briefly, 10 μg rAFAP-110 purified as mentioned above was incubated at 30° C. for 30 min with 0.5 μg recombinant PKCα in reaction buffer (20 mM Tris, pH 7.5, 5 mM MgCl₂, 0.2 mM CaCl₂, 20 μg/ml phosphatidylserine, and 2 μg/ml diolein) to which 1 μCi [γ-³²P]ATP was added. Reactions were analyzed by SDS-PAGE.

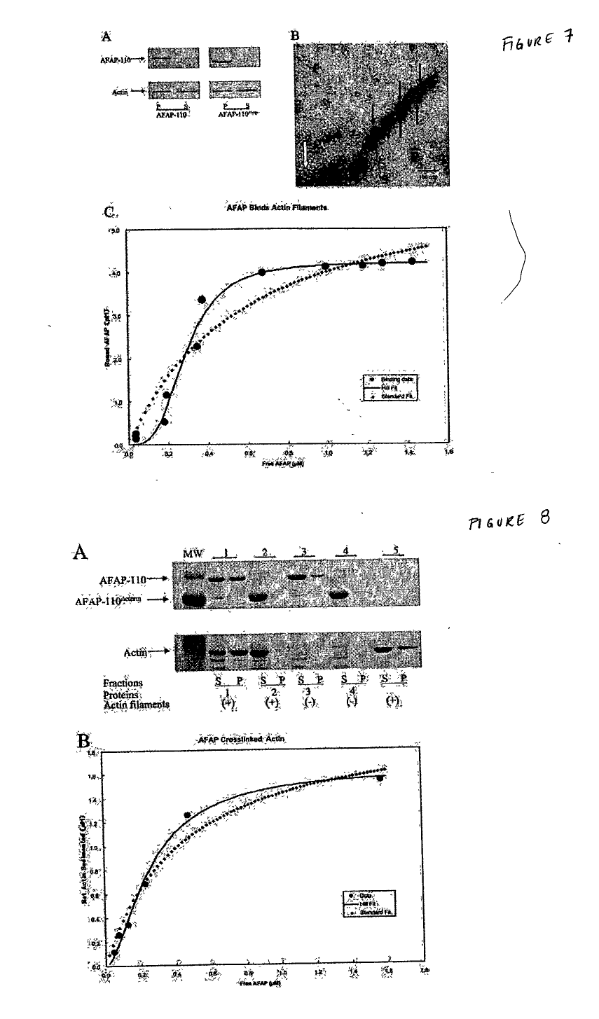

FIG. 10A demonstrates that PKCα phosphorylates rAFAP-110 in vitro. rAFAP-110 was incubated with or without recombinant PKCα in the presence of radiolabeled ATP. (1, PKCα (0.5 μg); 2, PKCα (0.5 μg)+rAFAP-110 (10 μg); 3, rAFAP-110 (10 μg)). Data are representative of three experiments. FIG. 10B demonstrates that AFAP-110 is phosphorylated in vivo in response to PMA treatment. C3H10T1/2 cells were serum-starved in phosphate-free media supplemented with radiolabeled ³²P-orthophosphate overnight and treated with 100 nM PMA, 100 nM 4α-PDD, or DMSO, the vehicle used. Cells were lysed after 15 min and AFAP-110 was immunoprecipitated using the antibody F1. Radiolabeled AFAP-110 was isolated by SDS-PAGE and subjected to phosphoamino acid analysis. As demonstrated in FIG. 10C, phosphotryptic analysis reveals common radioactive spots from AFAP-110 phosphorylated by PKC in vitro or phosphorylated in vivo in response to PMA ((C1) A representative tryptic map of rAFAP-110 phosphorylated in vitro by PKCα; (C2) a tryptic map of radiolabeled AFAP-110 purified from Cos-1 cells that were treated with PMA (100 nM, 1 h.). Black arrowheads, radioactive spots common between maps 1 and 2).

To test whether AFAP-110 could be phosphorylated in cells in response to PKC activation in vivo, serum-starved C3H10T1/2 fibroblasts labeled with ³²P-orthophosphate were stimulated with 100 nM PMA for 15 min to activate PKC. Cos-1 cells were maintained and transfected as previously described (Guappone and Flynn, 1997, supra). C3H10T1/2 cells were cultured as previously described (Qian et al., 2000, supra). Transient transfections of C3H10T1/2 cells were carried out using SuperFect (QIAGEN, Santa Clarita, Calif.) as previously described (Qian et al., 2000, supra).

Phosphoamino acid analysis of AFAP-110 immunoprecipitated from these cells indicates that AFAP-110 was hyperphosphorylated on serine and threonine residues in response to this treatment (FIG. 10B).

Phosphoamino Acid Analysis C3H10T1/2 cells were grown to 60% confluence in 100-mm culture dishes, serum starved overnight, and then stimulated with 100 nM PMA or 100 nM 4α-PDD for 15 min. Cells were washed twice with PBS and lysed with 1 ml RIPA buffer. mAb 4C3, 1.5 μl, was used to immunoprecipitate AFAP-110 from the lysate, which was isolated via SDS-PAGE. The radioactive band was excised from the gel and subjected to partial acid hydrolysis and phosphoamino acid analysis (Boyle et al., 1991, Methods Enzymol., 201:110-149). After running the isolated amino acids on 2D TLC, the plates were imaged using a Phosphorimager (Molecular Dynamics, Sunnyvale, Calif.). Spots were identified by running labeled phosphoserine, phosphothreonine, and phosphotyrosine markers. Relative intensity compared with background of radiation from spots was quantitated with ImageQuant software (Molecular Dynamics, Sunnyvale, Calif.).

Densitometry of the radioactive spots showed a 5.7-fold increase in phosphothreonine and a 3.9-fold increase in phosphoserine, relative to 4α-PDD treatment. Similar results were also obtained using Cos-1 cells transiently expressing AFAP-110. These data indicate that PMA treatment can induce ser/thr phosphorylation of AFAP-110 in vivo. Additionally, phosphotryptic mapping indicated the presence of similar radioactive spots upon analysis of AFAP-110 phosphorylated in vitro or AFAP-110 purified from PMA-treated Cos-1-expressing cells (FIG. 10C). These data do not discriminate between PKC or other activated ser/thr kinases as the enzyme responsible for phosphorylating AFAP-110. However, in agreement with previous predictions (Flynn et al., 1993, supra), these data indicate it is possible that AFAP-110 could represent a potential PKC substrate, in vivo.

Sequence analysis of both pleckstrin homology (PH) domains of AFAP-110 (Baisden et al., 2001 a, supra) revealed highest homology between the amino-terminal PH domain (PH1) and PH domains from β-spectrin and dynamin, which have been shown to forge interactions with PKC (Yao et al., 1997, supra; Rodriguez et al., 1999, Biochemistry, 38:13787-13794). The carboxy-terminal PH domain (PH2) was found to share highest homology with the PH domain from Btk, which also directs interactions with PKC (Yao et al., 1997, supra). The amino- and carboxy-terminal PH domains of AFAP-110 were expressed as GST-encoded fusion proteins (GST-PH1 and GST-PH2) and used to affinity-absorb cellular proteins from cell lysates as discussed herein.

Affinity-Absorption Assays

Both PH domains of AFAP-110 were amplified by PCR and subcloned from CMV-AFAP-110 into GEX-2T to create the GST-PH1 and GST-PH2 fusion proteins. Site-directed mutagenesis allowed for the in-frame deletion of residues 180-226, resulting in the generation of GST-PH1^{Δ180-226}. For phosphorylation assays, the immobilized fusion proteins were either phosphorylated with recombinant PKCα (CalBiochem) or left unphosphorylated. The rPKCα was washed away from the pellet, and then the rAFAP-110 was released using PreScission protease, according to manufacturer's instructions. The fusion proteins were dialyzed into kinase buffer for kinase assays. For Western blot analysis, the absorbates were analyzed by SDS-PAGE. For experiments involving serine/threonine kinase assays, the absorbates were washed five times with MTPBS (4.38 g NaCl, 1.14 g Na₂HPO₄, 0.24 NaH₂PO₄ in 500 ml H2O, pH 7.3)+1% Triton X-100 and then four times with TBS. The absorbate/bead slurry was subjected to a colorimetric PKA assay (Pierce, Rockford, Ill.) as per protocol.

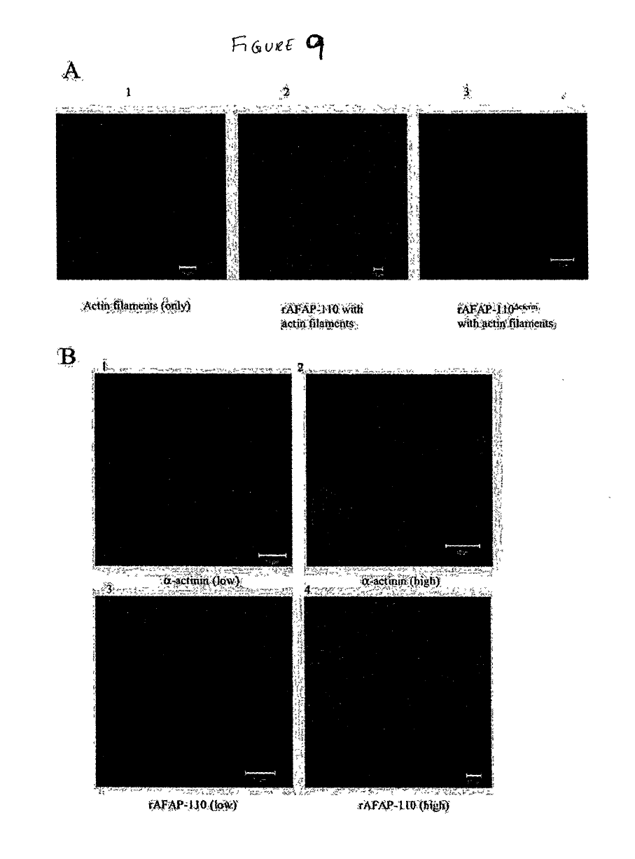

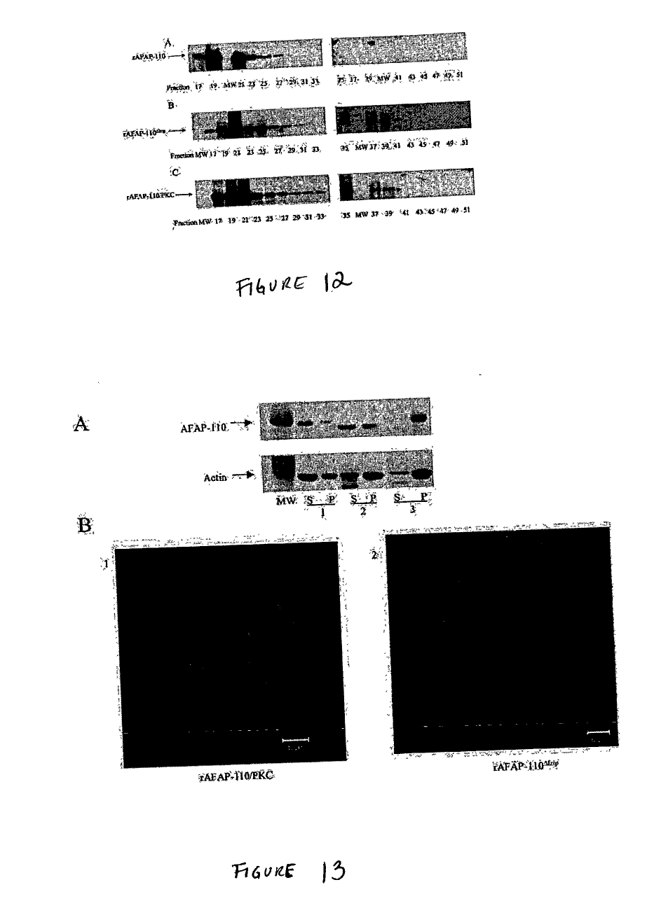

FIG. 11A demonstrates that GST-PH1 affinity absorbs α pan-PKC-immunoreactive proteins. Absorptions using fusion proteins as listed were analyzed by SDS-PAGE and Western blotted with a pan-PKC antibody. This blot is representative of three experiments. FIG. 14B demonstrates that GST-PH1 affinity-absorbs the classical PKC isoforms as well as PKCλ. Absorbates from affinity-absorptions using fusion proteins as listed were analyzed by SDS-PAGE and Western blotted with isoform specific PKC antibodies, as listed. Each blot is representative of two experiments. FIG. 1C present the results of coimmunoprecipitation of AFAP-110 and PKCα. Immunoprecipitation of GFP-AFAP-110 with mAb 4C3 results in the presence of an anti-Flag immunoreactive protein from cells expressing GFP-AFAP-110 and Flag-PKCα. Likewise, an anti-GFP immunoreactive protein is seen in anti-Flag immunoprecipitation from the same lysate, shown in C2.

It was demonstrated that absorbates from both PH domains could affinity-absorb ser/thr kinase activity, based on a colorimetric ser/thr kinase assay designed to detect activated PKC or PKA (Pierce). GST-PH1 appeared to absorb ser/thr kinase activity much more efficiently than GST-PH2. An additional fusion protein used in this study consisted of a deletion mutant of the amino-terminal PH domain (GST-PH₁^{Δ180-226}), which lacks consensus sequences associated with binding PKC (Yao et al., 1997, supra), and this fusion protein failed to absorb cellular ser/thr kinase activity. These GST-PH fusion proteins were also used to affinity-absorb from chick brain lysate in order to detect bound PKC. Using an antipan PKC antibody, which is immunoreactive with an epitope common to all PKC family members (Calbiochem), FIG. 11A demonstrates that four distinct proteins were affinity-absorbed by GST-PH1 but not by GST-PH2. Additional affinity-absorptions and Western blots were performed using antibodies specific for individual PKC isoforms. Antibodies specific for PKCα, β, δ, and λ/i isoforms were immunoreactive with proteins affinity-absorbed by GST-PH1, each of which had a Mᵣ equivalent to the known size of these PKC isoforms (FIG. 11B). The absorbates were devoid of protein bands immunoreactive with antibodies against PKCδ, ε, and ζ. Thus, the PH1 domain of AFAP-110 exhibits an ability to interact with at least four PKC isoforms. To determine if this interaction could be the result of direct binding, purified rPKCα was used in affinity-absorptions with GST-PH1 domain fusion proteins. It was found that GST-PH1 was able to absorb rPKCα with much higher efficiency than GST-PH2, the GST-PH1^{Δ180-226} mutant, or the GST protein alone. To determine whether AFAP-110 and PKC could be detected in complex with each other, coimmunoprecipitation experiments were used. FIG. 11C demonstrated that Flag-tagged activated PKCα coexpressed with GFP-tagged AFAP-110 will coimmunoprecipitate when using mAb 4C3, which is specific to the avian AFAP-110 construct (Qian et al., 1999, supra). Conversely, GFP-tagged AFAP-110 will coimmunoprecipitate with anti-Flag antibodies that bind to Flag-PKCα. Thus, AFAP-110 has the potential to form a direct interaction with the PKCα isoform.

### Example 7

### Either the Deletion of the Leucine Zipper Motif or PKC Phosphotylation Upregulates AFAP-110's Ability to Cross-Link Actin Filaments

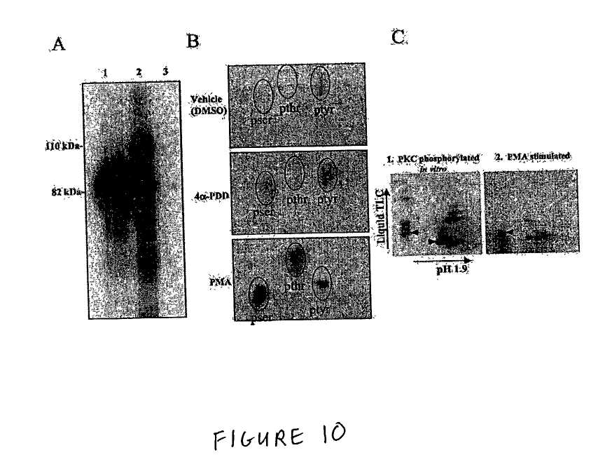

AFAP-110 may cross-link actin filaments through self-association. Therefore, changes in self-association may change AFAP-110's ability to cross-link actin filaments. The deletion of the leucine zipper motif reduces AFAP-110 multimers to dimers in 1% NP-40 buffer (Baisden et al., 2001 a, supra), similar to the effects on AFAP-110 in Src^{527F}-transformed cells (Qian et al., 1998, supra). To determine whether interactions with PKC could affect the ability of rAFAP-110 to cross-link actin filaments, gel filtration analysis was applied to determine how deletion of the leucine zipper motif or PKC phosphorylation of rAFAP-110 could affect rAFAP-110's ability to self-associate in vitro, within the context of F-actin binding buffer (FIG. 12).

FPLC Assays

Protein samples were fractionated on Superdex 200 (bed volume, 24 ml) at a flow rate of 0.3 ml/min. Ninety-five fractions were collected containing 250 μl each. The molecular weight markers were run as the controls. Fractions were collected and analyzed by Western blot analysis, as previously described (Qian et al., 1998, supra).

FIG. 12 demonstrates that either PKC phosphorylation or leucine zipper deletion destabilizes AFAP-110 multmerization. rAFAP-110 (A), rAFAP-110^{Δlzip} (B), and rAFAP-110/PKC (C) were separated by gel filtration. Fractions 17-51 were resolved by 8% SDS-PAGE gel. Western blot assays were applied using anti-AFAP-110 F1 antibody. The molecular weight markers were separated by gel filtration using FPLC and eluted as follows: thyglobulin (669 kDa) in fraction 25, ferritin (440 kDa) in fraction 30, catalase (232 kDa) in fraction 37, aldolase (158 kDa) in fraction 39, and albume (67 kDa) in fraction 44.

The results of FPLC separation by gel filtration show rAFAP-110 has one peak of elution at fraction 21 that corresponds to the molecular weight of 750 kDa (FIG. 12A), indicating rAFAP-110 forms large multimeric nonamers in vitro in actin buffer. rAFAP-110^{Δlzip} has two peaks of elution (FIG. 12B), one at fraction 21 and the other at fraction 37 (˜250 kDa), indicating that deletion of the leucine zipper motif may destabilize the multimeric AFAP-110^{Δlzip} complex. PKCα phosphorylation of rAFAP-110 also has two peaks of elution (FIG. 12C), one at fraction 21 and the other at fraction 37 (˜250 kDa), indicating a change similar to that of AFAP-110^{Δlzip}. The results demonstrate that deletion of the leucine zipper motif or PKCα phosphorylation may destabilize rAFAP-110 multimers, within the context of F-actin binding buffer.

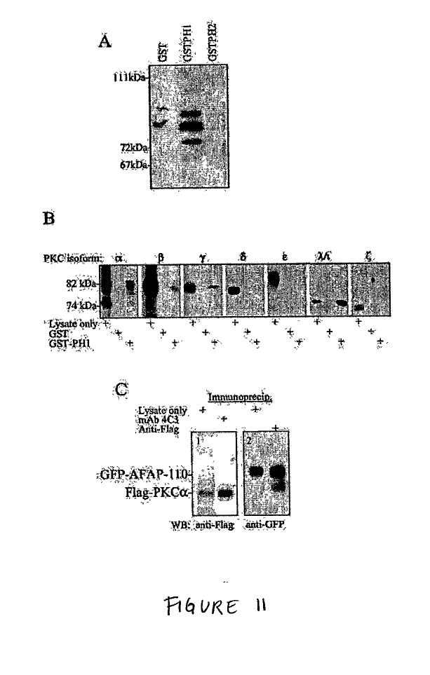

FIG. 13 demonstrates that either PKC phosphorylation or leucine zipper deletion increases AFAP-110's ability to cross-link actin filaments. In FIG. 13A. 0.5 μM purified rAFAP-110^{Δlzip} or 0.5 μM PKC phosphorylated rAFAP-110 was incubated with 2 μM actin filaments. After the incubation, the reactions were centrifuged at 20,800×g. Both supernatants (S) and pellets (P) were applied to SDS-PAGE gel, followed by Coomassie stain. The data are representative of two different experiments. (A1) rAFAP-110; (A2) rAFAP-110^{Δlzip}; (A3) PKC phosphorylated rAFAP-110). FIG. 13B presents confocal microscopy images. 0.5 μM purified rAFAP-110^{Δlzip} or 0.5 μ/M PKC phosphorylated rAFAP-110 was incubated with 10% rhodamine-phalloidin-labeled actin filaments (2 μM). After the incubation, the reactions were observed with a Zeiss confocal microscope.

Immunofluorescence

For PKC activation with PMA, C3H10T1/2 cells were transfected with plasmids encoding AFAP-110. Twenty-four hours after transfections, the cells were serum starved overnight. PMA at 100 nM was used to activate PKC for 60 min. Cells were fixed and permeablized as previously described (Qian et al., 1998, supra). After washing, cells were labeled with BODIPY 650/665 phalloidin (Molecular Probes, Eugene, Oreg.) and 4C3 antibody for 20 min. Cells were washed again and then labeled with ALEXA 488 (Molecular Probes) for 25 min. Cells were washed and mounted on slides with Fluoromount (Fisher, Pittsburgh, Pa.). For serum induction experiments, C3H10T1/2 cells were serum starved overnight, and then serum-complete media was added. Cells on coverslips were washed and fixed at 15-min time points >2 h. Polyclonal antibody F1 and TRITC-labeled anti-rabbit secondary antibody were used to visualize endogenous AFAP-110, with washings as above. BODIPY 650/665 phalloidin was used to visualize actin. A Zeiss LSM 510 microscope (Thornwood, N.Y.) was used to gather images, which were recolored from grayscale. Scale bars were generated and inserted by LSM 510 software (Carl Zeiss, Thornwood, N.Y.).

The results of low-speed centrifugation demonstrated that either deletion of the leucine zipper motif or PKCα phosphorylation increased the ability of rAFAP-110 to coprecipitate actin filaments (FIG. 13A), indicating that both deletion of the leucine zipper motif or phosphorylation by PKCα induced more extensive F-actin cross-linking capability by AFAP-110. Confocal microscopy of rhodamine-phalloidin-labeled actin filaments in the presence of PKCα phosphorylated rAFAP-110 showed large aggregates of fluorescence (FIG. 13B1), similar to the pattern induced by rAFAP-110^{Δlzip} (FIG. 13B2) and distinct from the varicose pattern produced by native rAFAP-110 (see FIG. 9A). Electron microscopy revealed that both PKCα phosphorylated rAFAP-110, and the leucine zipper deletion mutant induced extensive aggregation of actin filaments.

### Example 8

### AFAP-110 Mediates the Effects of PKC on the Structural Changes of the Cells

AFAP-110 has been previously demonstrated to exist in dynamic actin structures in response to Src activation (Qian et al., 1998, supra). FIG. 14 demonstrates that AFAP-110 mediates the effects of PKC on actin filaments. FIG. 14A demonstrates that AFAP-110 localizes to dynamic actin structures seen upon PMA stimulation. Immunofluorescence labeling and imaging was carried out as described. C3H10T1/2 cells expressing AFAP-110 were treated with either 100 nM PMA (A1-A3) or 100 nM 4α-PDD (A4-A6) for 15 min after overnight serum starvation. AFAP-110 is represented in red (A1 and A4), whereas actin is represented in green (A2 and A5). (A3 and A6) Overlap images of AFAP-110 and actin. Arrows indicate colocalization of AFAP-110 with dynamic actin structures: white arrows, filopodia; gray arrows, lamellipodia; black arrows, actin filaments in the top panel and disrupted actin filaments/rosettes in the bottom panel. FIG. 14B demonstrates that AFAP-110 localizes to lamellipodia/filopodia seen upon expression of active PKC. Immunofluorescence labeling and imaging were carried out as described in Experimental Procedures. C3H10T1/2 cells coexpressing GFP-AFAP-110 (B1) and myristoylated, Flag-tagged PKC were labeled to show Flag-tagged PKC (B2) and actin (B3). Arrows indicate colocalization of AFAP-110 with dynamic actin structures: white arrows label filopodia and black arrows label lamellipodia. FIG. 14C demonstrates that AFAP-110 mediates the effects of PKC on the changes of cell structures. Immunofluorescence labeling and imaging were carried out as described. C3H10T1/2 cells were transfected with pCMV-GFP-AFAP-110 (C1-C6) and pCMV-GFP-AFAP^{Δ180-226} (C7-C9). PMA at 100 nM was used to stimulate the cells for 1 h. C1, C4, and C7; green GFP fusion protein images; C2, C5, and C8; X actin images; C3, C6, and C9, overlap images of GFP fusion protein and actin images.

FIG. 14 confirms that AFAP-110 similarly exists in these structures detected in serum-starved fibroblasts upon treatment with PMA, a PKC activator. Lamellipodia, filopodia, and rosettes appear in these cells upon PMA treatment, whereas stress filaments become less apparent. These changes occur within 15 min and largely revert by 2 h after treatment with 100 nM PMA. By 6 h after treatment, the cells have completely reverted to a quiescent phenotype and AFAP-110 retains its localization with actin filaments over this time course. The cells shown represent transient transfections of C3H10T1/2 cells, and similar results are seen with endogenous AFAP-110 in these cells. Active PKCα was overexpressed in cells. FIG. 14B shows that AFAP-110 similarly exists in these structures seen in fibroblasts upon coexpression of active PKCα. GFP-tagged AFAP-110 and Flag-tagged myristoylated PKCα were coexpressed in C3H10T1/2 fibroblasts. Anti-Flag antibodies were used to label Flag-Myr-PKCα (FIG. 14B2), and FITC-phalloidin was used to label actin filaments (FIG. 14B3). Lamellipodia and filopodia appear in these cells, whereas stress filaments become less apparent. GFP-AFAP-110 is found in motility structures, as designated with white arrows (filopodia) and black arrows (lamellipodia). These results indicate AFAP-110 is properly positioned to play a role in the formation of these structures in cells in response to PKC activation.

PKCα has the capability to phosphorylate AFAP-110 and the potential to bind AFAP-10 via its amino terminal PH domain (PH1; see FIG. 11). A deletion mutant of the PH1 domain of AFAP-110, AFAP-110^{Δ180-226}, was subcloned into the pGFP vector. Both pGFP-AFAP-110 and pGFP-AFAP-110^{Δ180 -226} were transiently transfected into C3H10T1/2 cells. GFP-AFAP-110 colocalized with actin filaments and the cell membrane (FIG. 14, C1-C3). PMA, a PKC activator, was applied to stimulate these cells. It was found that PMA stimulation of GFP-AFAP-110 cells disrupted the integrity of actin filaments, induced the formation of cell motility structures, and positioned GFP-AFAP-110 into these structures (FIG. 14, C4-C6). However, GFP-AFAP^{Δ180-226} appeared to interfere with the effects of PMA on actin filaments, whereby GFP-AFAP^{Δ180-226} was evenly localized upon well-formed actin filaments and the cell membrane (FIG. 14, C7-C9). There results indicate that AFAP-110 can play a role in mediating the effects of PMA on actin filaments.

### Example 9

### Deletion of the Leucine Zipper Motif Enables AFAP110 to Alter Actin Filament Integrity

The expression of AFAP-110^{Δlzip} results in a cell phenotype which resembles Src-transformed cells, while overexpression of wild-type AFAP-110 has no effect on actin structures (Qian et al., 1998, supra; Qian et al., 2000, supra).

Cell Culture

Cos-1 cells were maintained and transfected as previously described (Guappone and Flynn, 1997, supra). C3H10T1/2 cells were cultured as previously described (Qian et al., 2000, supra). Transient transfections of C3H10T1/2 cells were carried out using Effectene (Qiagen) as per manufacturers instructions. Transient transfections of Cos-1 cells employed the CalPhos transfection kit (Clontech).

Immunofluorescence

C3H10T1/2 cells were transiently transfected, as above. 48 hours after transfections, the cells were serum-starved overnight. Cells were fixed and permeabilized as previously described (Qian et al., 1998). After washing, cells were labeled for 30 minutes. For actin labeling, a 1:1000 dilution of rhodamine-phalloidin was used. Antibody concentrations used were as follows: 4C3, 0.6 μg/ml in 5%BSA; Anti-HA, 4 μg/ml in 5%BSA; anti-phosphotyrosine, 2.5 μg/ml in 5% BSA; anti-phospho-Src (Y⁴¹⁶) labeling, 5 μl/ml in 5%BSA. Both polyclonal phospho-specific antibodies were visualized by incubating the cells for 30 minutes with anti-rabbit-Alexa 633 (5 μl/ml in 5%BSA) after washing off primary antibody. Monoclonal antibody 4C3 was visualized using Alexa 488 anti-mouse secondary antibody (5 μl/ml in 5%BSA). Cells were washed and mounted on slides with Fluoromount (Fisher). A Zeiss LSM 510 microscope was used to gather grayscale images, which represent confocal slices of about 2 μM in thickness. Scale bars were generated and inserted by LSM 510 software.

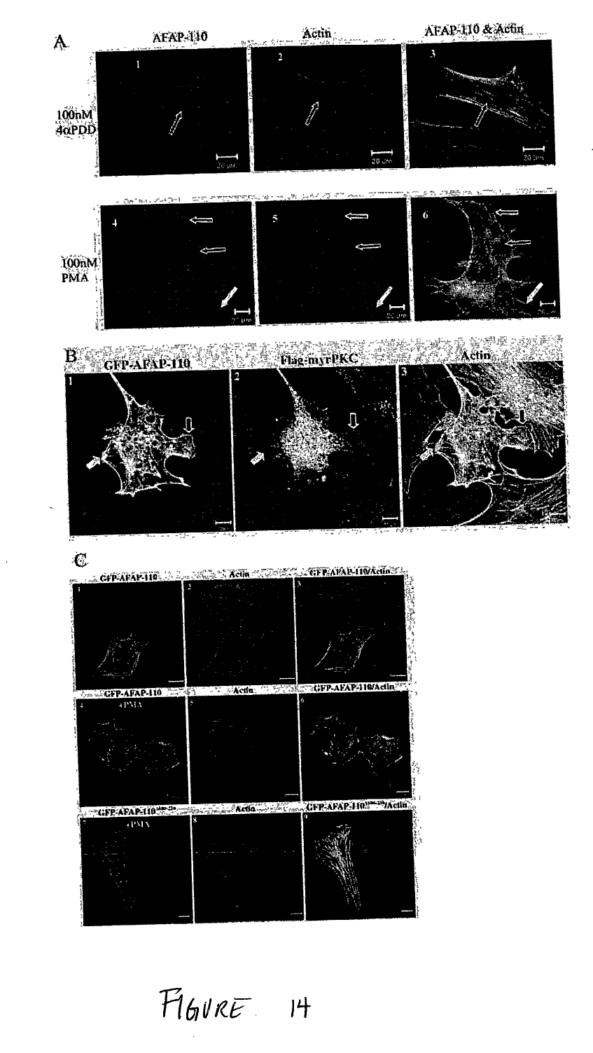

Cells expressing AFAP-110^{Δlzip} consistently displayed actin-rich rosettes in place of actin filaments, as well as actin-rich lamellipodia. Filopodia are also detected in some of these cells. GFP-tagged AFAP-110 expressed in C3H10T1/2 cells co-localizes with actin filaments and the cell membrane (FIG. 15, panels A and B), while GFP-AFAP-110^{Δlzip} co-localizes with and induces the formation of actin filament rich-rosettes (white arrowheads, panels C and D) and lamellipodia (gray arrowheads, panels C and D). FIG. 15 demonstrates that AFAP-110^{Δlzip} expression results in the disruption of actin filaments and formation of rosettes. GFP-AFAP-110 (panel A) colocalizes with actin filaments (panel B) in C3H10T1/2 cells. GFP-AFAP-110^{Δlzip} (panel C) localizes to actin rosettes (white arrowheads) seen in these cells, which are not present in surrounding cells (panel D). These transfected cells also exhibit increased levels of lamellipodia-like structures (gray arrowheads), compared to surrounding, non-transfected cells. Representative images are shown (>100 cells examined).

Lamellipodia were noted as actin-rich structures at the cell membrane of variable size. GFP expressed alone in these cells will not co-localize with actin filaments or affect cytoskeletal structures (Qian et al., 2000, supra). Similar results were also detected in Cos-1 fibroblasts (data not shown). These data indicate that the leucine zipper plays a regulatory role for AFAP-110 and indicate that AFAP-110 also has an intrinsic ability to reposition actin filaments into rosettes and generate lamellipodia formation when the leucine zipper is no longer present.

### Example 10

### Expression of AFAP-110^{Δlzip} Directs an Increase in Cellular Tyrosine Phosphorylation

Phosphoamino acid analysis was performed on AFAP-110 and AFAP-110^{Δlzip} from transfected Cos-1 cells.

Phosphoamino Acid Analysis

Cos-1 cells were grown to 60% confluence in 100 mm culture dishes, and transiently transfected. After 48 hours, the cells were serum starved overnight in phosphate free media supplemented with 2.5 mCi ³²P-orthophosphate (Amersham). Cells were washed twice with PBS and lysed with 1 ml RIPA buffer, as previously described (Qian et al., 1998, supra). One and one-half μl monoclonal antibody 4C3 ascites was used to immunoprecipitate AFAP-110 from the lysate, which was isolated via SDS-PAGE. The radioactive band was excised from the gel and subjected to partial acid hydrolysis and phosphoamino acid analysis (Boyle et al., 1991, Methods Enzymol, 201:110-149). During this analysis, samples were monitored with a scintillation counter during each step to check for loss, which was similar for both AFAP-110 and AFAP-110^{Δlzip}. After running the isolated amino acids on 2D TLC, the plates were imaged using a Phosphorimager (Molecular Dynamics). Spots were identified by running labeled phosphoserine, phosphothreonine and phosphotyrosine markers. Relative intensity compared to background of radiation from spots was quantitated with ImageQuant software.

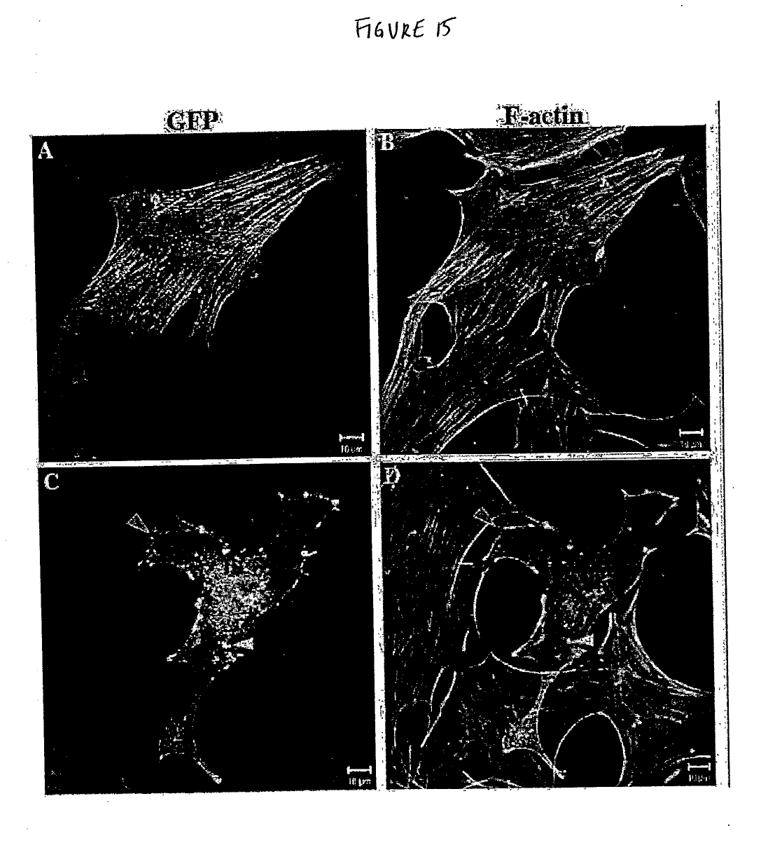

FIG. 16 demonstrates that FAP-110^{Δlzip} is hyperphosphorylated upon expression in Cos-1 cells. In FIG. 16A phosphoamino acid analysis (Boyle et al., 1991, supra) of radio-labeled AFAP-110 and AFAP-110^{Δlzip} immunoprecipitated from transfected Cos-1 cells (as in FIG. 15) demonstrates that AFAP-110^{Δlzip} (right panel) is hyperphosphorylated compared to AFAP-110 (left panel). ImageQuant analysis of spot intensity revealed 1.6 fold, 2.6 fold and 2.4 fold increases in serine, threonine, and tyrosine phosphorylation, respectively. FIG. 15B demonstrates the results of Western blot analysis of mAb 4C3 immunoprecipitations followed by rabbit anti-phosphotyrosine immunoblotting of AFAP-110 (lane 1), AFAP-110 co-expressed with Src^{527F} (lane 2) and AFAP-110^{Δlzip} (lane 3) from Cos-1 cell lysate. (C) The same western blot from B was stripped and reprobed with mAb 4C3 to demonstrate protein loading levels (Lane: 1—AFAP-110, 2—AFAP-110/Src^{527F}, 3—AFAP-110^{Δlzip}). The western blot shown is representative of three independent experiments.

Under equal loading conditions, over-expressed AFAP-110^{Δlzip} contains elevated levels of serine, threonine, and tyrosine phosphorylation, compared to over-expressed wild-type AFAP-110 (FIG. 16A). Phosphorylation was increased by 1.6 fold, 2.6 fold and 2.4 fold, respectively, as determined by scanning densitometry. An additional experiment showed similar results, with incorporation of phosphate into this mutant increased over wild-type AFAP-110 by 2.6, 3.6, and 3.5 fold for serine, threonine, and tyrosine, respectively. Anti-phosphotyrosine antibodies were used for western blot analysis of immunoprecipitated proteins to confirm the increase in tyrosine phosphorylation levels of AFAP-110^{Δlzip} relative to AFAP-110 (FIG. 16B). Also, GFP-tagged forms of AFAP-110 demonstrated levels of phosphotyrosine similar to their untagged counterparts (data not shown). These data indicate that AFAP-110^{Δlzip} becomes hyperphosphorylated on serine, threonine, and tyrosine residues, relative to wild-type AFAP-110.

### Example 11

### AFAP-100^{Δlzip} Expression Activates Cellular Tyrosine Phosphorylation

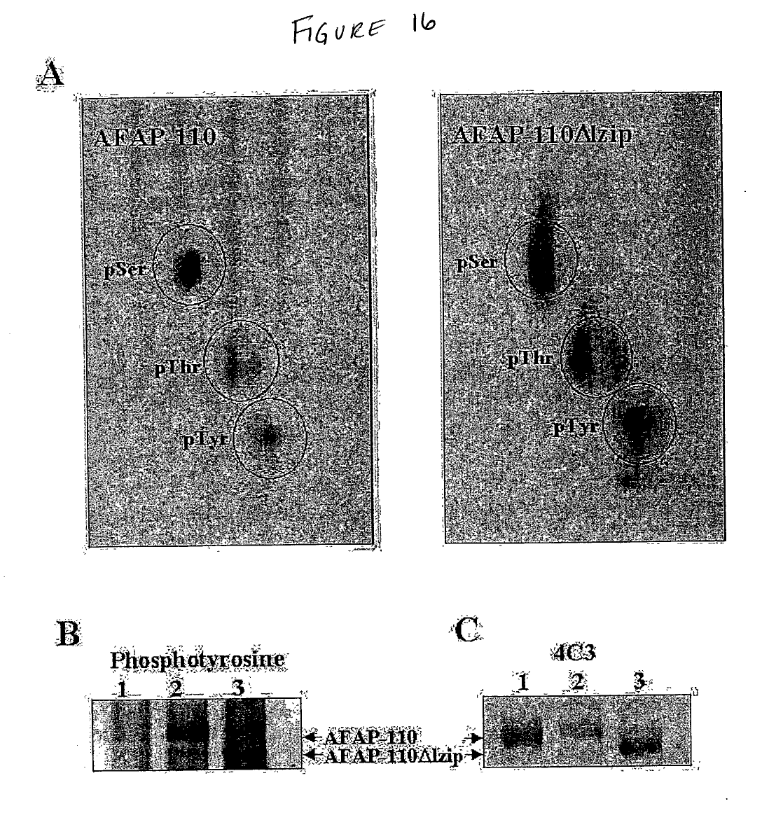

The increases in tyrosine phosphorylation of AFAP-110^{Δlzip} indicated that it may induce the activation of cellular kinases. Anti-phosphotyrosine antibodies were used to determine if total cellular tyrosine phosphorylation was increased and to which subcellular sites tyrosine substrates were localized in these cells. FIG. 17 demonstrates that AFAP-110^{Δlzip} activates cellular tyrosine phosphorylation. FIG. 17A presents immunofluorescence images of transfected C3H10T1/2 cells expressing GFP-AFAP-110 (panel A) and GFP-AFAP-100^{Δlzip} (panel D) co-labeled with rhodamine-phalloidin (panels C and F) and rabbit-anti-phosphotyrosine antibodies (panels B and E). White arrowheads in panels D-F indicate the co-localization of increased phosphotyrosine labeling with GFP-AFAP-110^{Δlzip} and F-actin at the cell membrane. Representative images are shown (>100 cells examined).

FIG. 17 demonstrates the results obtained using immunofluorescence of transiently transfected C3H10T1/2 cells. Anti-phosphotyrosine immunolabeling revealed no significant change in phosphotyrosine levels in GFP-AFAP-110 transfected cells; however, an overall increase in tyrosine phosphorylation throughout the cell, as well as a co-localization of phosphotyrosine containing proteins with GFP-AFAP-110^{Δlzip} and actin-rich structures at the cell membrane, was evident in GFP-AFAP-110^{Δlzip} transfected cells, relative to untransfected cells (white arrowheads, panels D-F). Similar results were seen in Cos-1 cells (data not shown). It is noteworthy that some labeling of tyrosine phosphorylated proteins with the anti-phosphotyrosine antibody was apparent along the cell membrane and in actin-rich structures in non-transfected cells; however, the intensity of immunolabeling was significantly lower than AFAP-₁₁₀^{Δlzip} expressing cells. These structures appeared to represent lamellipodia, which are known to contain tyrosine phosphorylated proteins and whose formation and extension involve Src activation (Boschek et al., 1981, Cell, 24:175-184; Schwartzberg et al., 1997, Genes Dev., 11:2835-2844). The levels of immunostaining for these tyrosine phosphorylated proteins was also noticeably lower in cells expressing wild-type AFAP-110, which was equivalent to untransfected cells (FIGS. 17A-C). These results demonstrate an increase in cellular tyrosine phosphorylated substrates along the cell membrane and within actin-rich structures in response to the expression of GFP-AFAP-110^{Δlzip}.

### Example 12

### AFAP-110^{Δlzip} Expression Results in the Specific Activation of Src Family Kinases

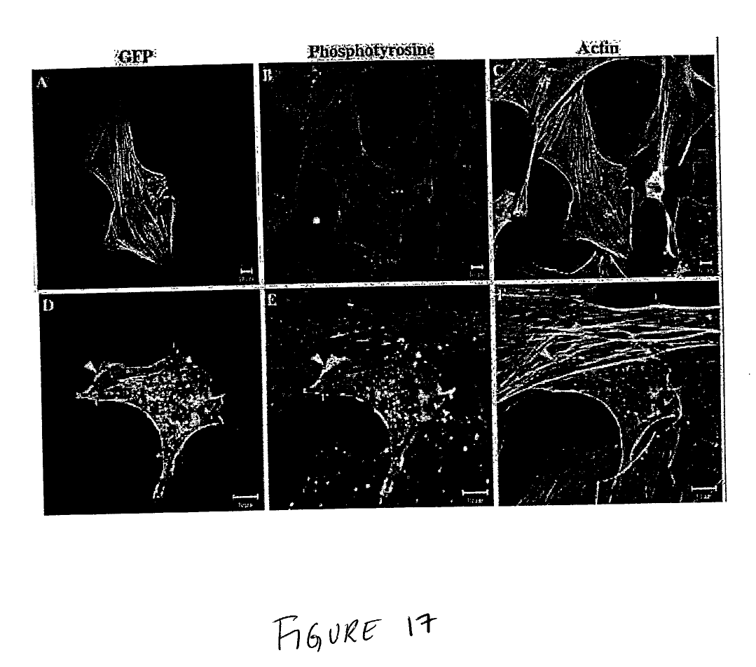

Specific Src family activation-state antibodies were used to determine if AFAP-110^{Δlzip} affected autophosphorylation of Src family kinases, an indicator for activation of these tyrosine kinases. FIG. 18 demonstrates that AFAP-110^{Δlzip} activates Src family kinases. Anti-phospho-Src (Y⁴¹⁶) labeling (panels B and E) of Cos-1 cells expressing GFP-AFAP-110 (panel A) and GFP-AFAP-110^{Δlzip} (panel D) co-labeled with rhodamine-phalloidin (panels C and F). White arrowheads in panels D-F indicate the co-localization of increased phospho-Src (Y⁴¹⁶) labeling with GFP-AFAP-110^{Δlzip} and F-actin at a structure resembling a lamellipodium. Representative images are shown (>100 cells examined).

Immunofluorescence analysis of C3H10T1/2 cells expressing GFP-AFAP-110 revealed no significant increase in immunoreactivity compared to untransfected cells, as discussed herein. However, co-localization of these activated Src family members with GFP-AFAP-110^{Δlzip} and actin-rich structures at the cell membrane was evident (white arrowheads, panels D-F). These cells also displayed an increase in fluorescence intensity throughout the cell for the anti-Src activation state antibody, compared to surrounding, non-transfected cells. This result correlated well with GFP-AFAP-110^{Δlzip} expression levels. Similar results were seen in Cos-1 cells, in which the colocalization of GFP-AFAP-110^{Δlzip} with actin and activated Src occurred at membrane ruffles and lamellipodia-like structures (data not shown). This Src family activation state antibody showed some labeling of protein in non-transfected cells, mostly in what appears to be perinuclear structures. These results indicate that expression of GFP-AFAP-110^{Δlzip} can direct autophosphorylation of one or more Src family kinases throughout the cell and specifically in actin-rich structures that co-localize with GFP-AFAP-110^{Δlzip} along the cell membrane.

### Example 13

### A Mutation Which Alters the SH3-Binding Motif in AFAP-110 Prevents AFAP-110^{Δlzip} From Activating Tyrosine Kinases or Altering Actin Filament Integrity

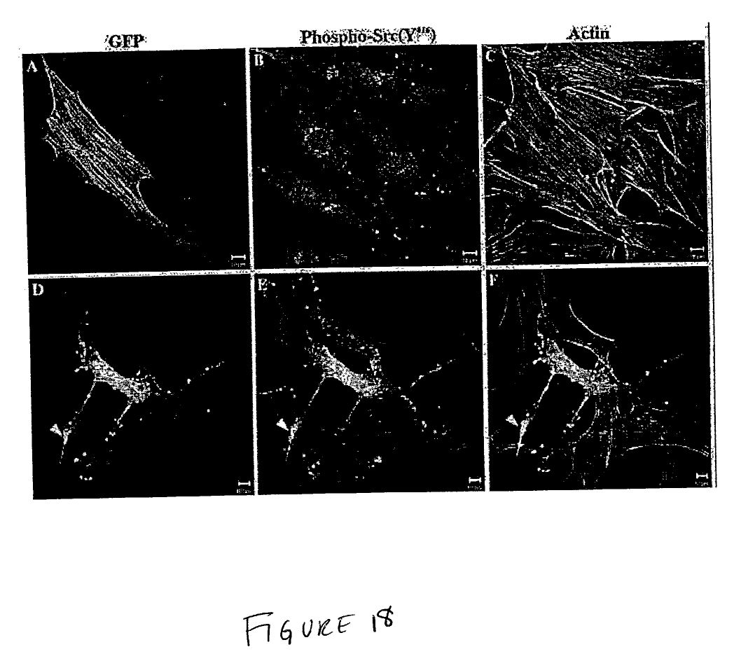

Some SH3-binding partners can activate Src-family kinases via SH3 binding (Moarefi et al., 1997, Nature, 385:650-653). AFAP-110 is also an SH3-binding partner for cSrc, cFyn or cLyn (Flynn et al., 1993, supra; Guappone and Flynn, 1997, supra). AFAP-110^{P71A/Δlzip} was created, which contains an additional point mutation in the SH3-binding motif that has been shown to abrogate SH3 interactions with Src, and also prevents Src/AFAP-110 stable complex formation (Guappone and Flynn, 1997, supra). AFAP-110^{P71A/Δlzip} was expressed in C3H10T1/2 cells to examine the ability of this mutant to activate Src family kinases. FIG. 19 demonstrates that abrogation of SH3 binding inhibits the cytoskeletal rearrangement and activation of Src family kinases by AFAP-110^{Δlzip}. C3H10T1/2 cells expressing AFAP-110^{71A/Δlzip} (panels A and D) were labeled as above with anti-phosphotyrosine (panel B) and anti-phospho-Src (Y⁴¹⁶) (panel E) antibodies. Panels C and F show normal actin filaments, labeled with rhodamine-phalloidin. (>100 cells examined).

This mutant failed to induce an increase in cellular tyrosine phosphorylation (FIG. 19, panels A-C). Additionally, AFAP-110^{P71A/Δlzip} failed to increase cellular immunoreactivity with Src family kinase activation state antibodies (panels D-F). These cells displayed no increase in labeling by these phosphorylation state antibodies, compared to non-transfected surrounding cells, unlike those expressing GFP-AFAP-110^{Δlzip} (FIG. 17). AFAP-110^{P71A/Δlzip} also failed to alter actin filaments or induce lamellipodia formation, while maintaining co-localization with actin filaments. Thus, SH3 domain binding by AFAP-110^{Δlzip} appears to be necessary for its ability to activate cellular tyrosine phosphorylation, Src family kinases and cytoskeletal rearrangements.

### Example 14

### The Integrity of the PH Domain of AFAP-110 is Also Required for AFAP-110^{Δlzip}-Directed Increases in Cellular Tyrosine Phosphorylation, Src Family Activation and Cytoskeletal Rearrangements

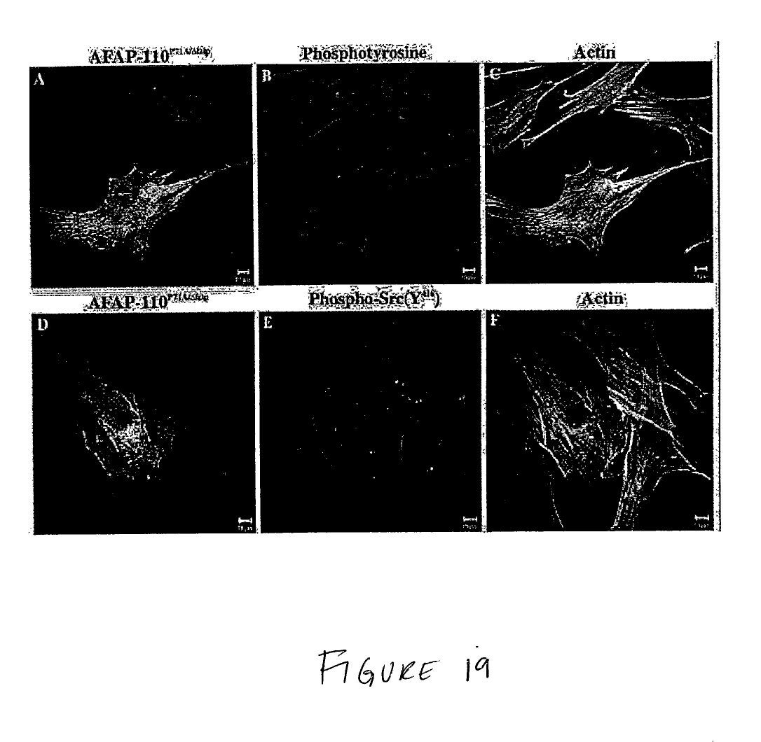

GFP-AFAP-110^{Δ180-226/Δlzip} contains an additional deletion of 46 amino acids in the amino-terminal PH domain, which is predicted to disrupt the function of this domain. FIG. 20 demonstrates that deletion of part of the amino-terminal PH domain of AFAP-110 inhibits the cytoskeletal rearrangement and activation of Src family kinases by AFAP-110^{Δlzip}. C3H10T1/2 cells expressing AFAP-110^{Δ180-226/Δlzip} (panels A and D) were labeled as above with anti-phosphotyrosine (panel B) and anti-phospho-Src (Y⁴¹⁶) (panel E) antibodies. Panels C and F show normal actin filaments, labeled with rhodamine-phalloidin. (>100 cells examined).

FIG. 20 demonstrates the results obtained upon labeling C3H10T1/2 cells expressing GFP-AFAP-110^{Δ180-226/Δlzip} with both anti-phosphotyrosine (panels A-C) and Src family activation state antibodies (panels D-F). This mutant fails to induce cytoskeletal rearrangements, as actin filaments (panels C and F) are abundant and the mutant (panels A and D) colocalizes with these structures. Additionally, this mutant fails to induce cellular tyrosine hyperphosphorylation (panel B) or Src family kinase activation (panel E), compared to surrounding, non-transfected cells. Also, AFAP-110^{Δ180-226/Δlzip} had reduced levels of phosphotyrosine compared to AFAP-110^{Δlzip} (data not shown). Similar results were seen in Cos-1 cells. Thus, the amino-terminal PH domain of AFAP-110 also plays an important role in the ability of AFAP-110^{Δlzip} to activate Src and alter actin filament integrity.

### Example 15

### AFAP-110^{Δlzip} Alters Actin Filaments in a Rho-Dependent Fashion

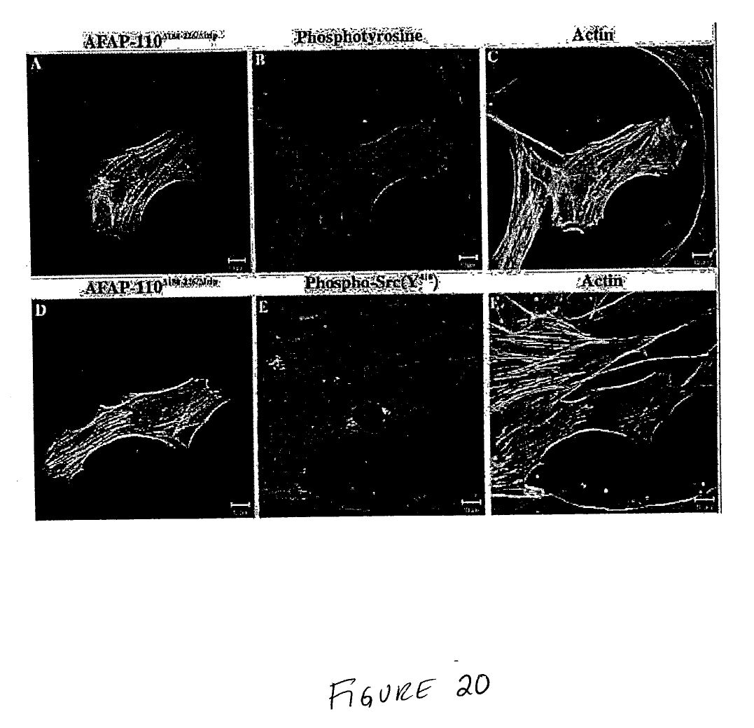

The GTP-binding protein, Rho, plays an important role in effecting changes in actin filaments downstream of Src. Activated Rho can restore actin filament organization, in Src-transformed cells (Mayer et al., 1999, Oncogene, 18:2117-2128), and cSrc activation requires the inactivation of Rho, by enhancing GTPase activity, to induce actin filament dissolution (Fincham et al., 1999, J. Cell Sci., 112(Pt 6), 947-956). GFP-AFAP-110^{Δlzip} was co-expressed in C3H10T1/2 cells with dominant positive Rho (RhoA^{V14}) to determine if GFP-AFAP-110^{Δlzip} may exert its affects on actin filaments in a Rho-dependent fashion. FIG. 21 demonstrates that RhoA^{V14} overcomes the ability of AFAP-110^{Δlzip} to alter actin filaments. Representative immunofluorescence images of transfected C3H10T1/2 cells expressing GFP-AFAP-110 (panel A) or GFP-AFAP-110^{Δlzip} (panel D) and HA-tagged RhoA^{V14} (panels B and E) demonstrate abundant actin filaments (panels C and F) and both AFAP-110 constructs co-localize with these structures. GFP-AFAP-110^{Δlzip} colocalizes with actin-rich structures at the cell membrane, as denoted by the white arrowheads, panels D and F (>100 cells examined).

Cells expressing GFP-AFAP-110 and RhoA^{V14} were consistently filled with well-developed actin filaments (FIG. 21, panels A-C). Similar results were seen in Cos-1 cells (data not shown). Interestingly, in GFP-AFAP-110^{Δlzip} expressing cells, RhoA^{V14} largely overcomes the ability of GFP-AFAP-110^{Δlzip} to reposition actin filaments into rosettes (panels D-F), consistent with previous results that demonstrated significantly increased amounts of F-actin (Ridley and Hall, 1992, Cell, 70:389-399). It is noteworthy that GFP-AFAP-110^{Δlzip} is more prominently represented along the cell membrane and within small membrane protrusions, while less well associated with stress filaments, compared to AFAP-110 co-expressed with RhoA^{V14}. Additionally, these cells display some actin rosettes. Both GFP-AFAP-110^{Δlzip} and actin are found in these structures, albeit at levels significantly lower than in cells not expressing RhoA^{V14}. These results demonstrate that the ability of AFAP-110^{Δlzip} to reposition actin filaments into rosettes occurs in a Rho-dependent fashion, and, like Src, may require the down-regulation of Rho to complete this function. To confirm that AFAP-110^{Δlzip} activation of Src is responsible for these changes in actin filaments and not a consequence of changes in actin filament integrity, C3H10T1/2 cells were co-transfected as above and examined for tyrosine phosphorylation levels and Src family kinase activation.

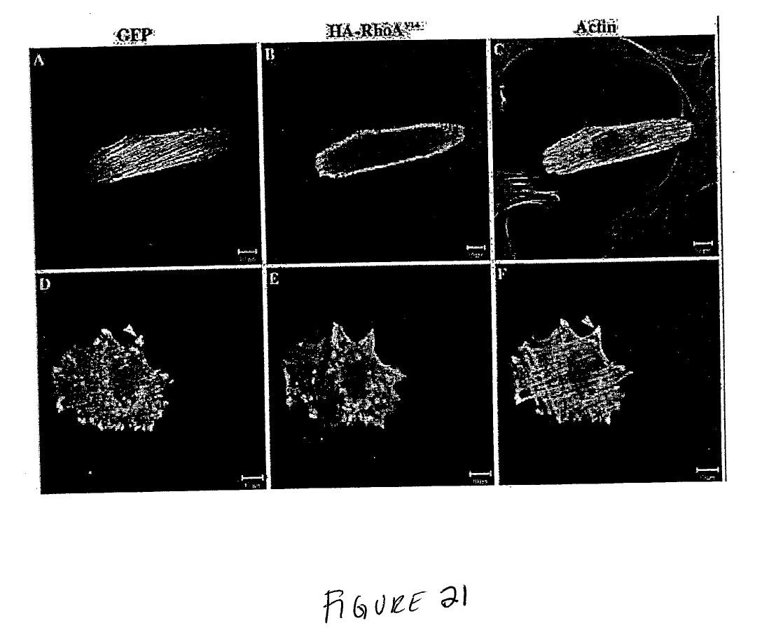

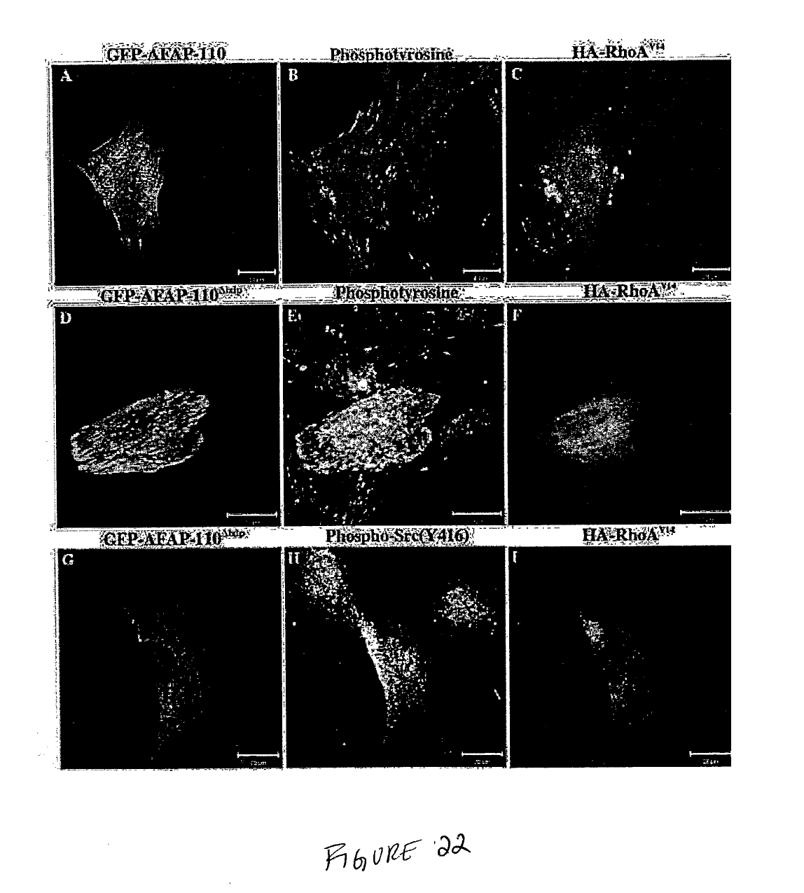

FIG. 22 demonstrates that AFAP-110 activates cellular tyrosine phosphorylation and Src family kinase activation in the presence of RhoA^{V14}. Representative immunofluorescence images of transfected C3H10T1/2 cells expressing GFP-AFAP-110 (panel A) or GFP-AFAP-110^{Δlzip} (panels D and G) and HA-tagged RhoA^{V14} (panels C, F and I). These were labeled with anti-phosphotyrosine (panels B and E) or anti-phospho-Src (Y⁴¹⁶) (panel H).

FIG. 22 compares the activation of tyrosine phosphorylation by GFP-AFAP-110 (panels A-C) and GFP-AFAP-110^{Δlzip} (panel D-F) in the presence of RhoA^{V14}. Expression of GFP-AFAP-110^{Δlzip} results in the increase of tyrosine phosphorylation in the presence of RhoA^{V14}. GFP-AFAP-110 fails to induce tyrosine phosphorylation in the presence of RhoA^{V14}, in agreement with previous figures. Thus, the repositioning of actin filaments into rosettes and the induction of lamellipodia formation by AFAP-110^{Δlzip} may proceed largely through indirect mechanisms that permit it to activate Src-family kinases and the subsequent activation of downstream signals which ultimately alter actin filament organization.

### Example 16

### Insoluble AFAP-110

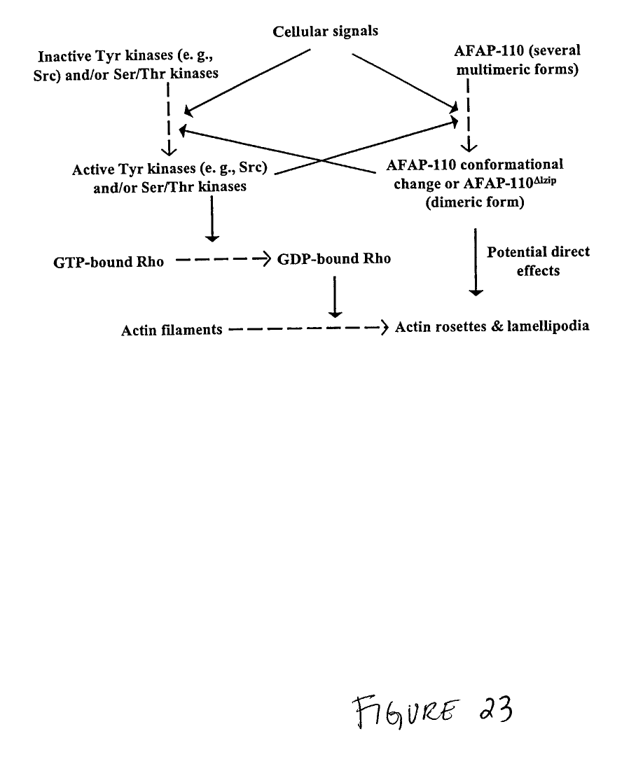

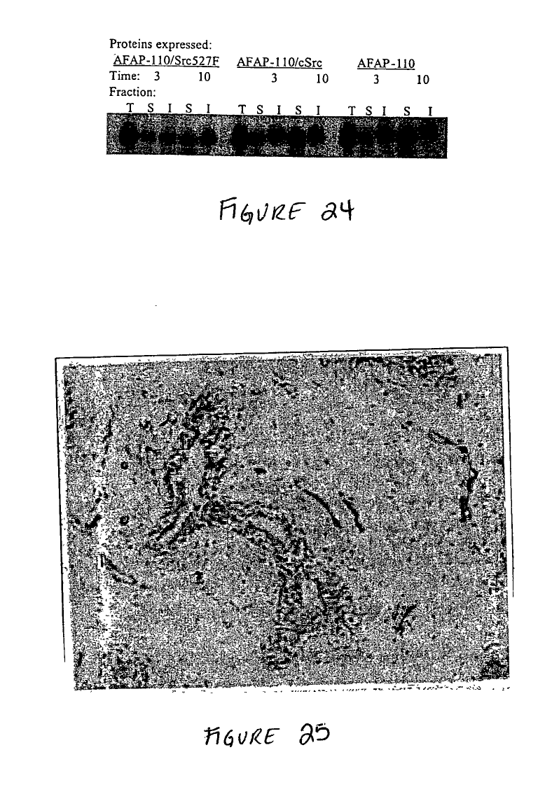

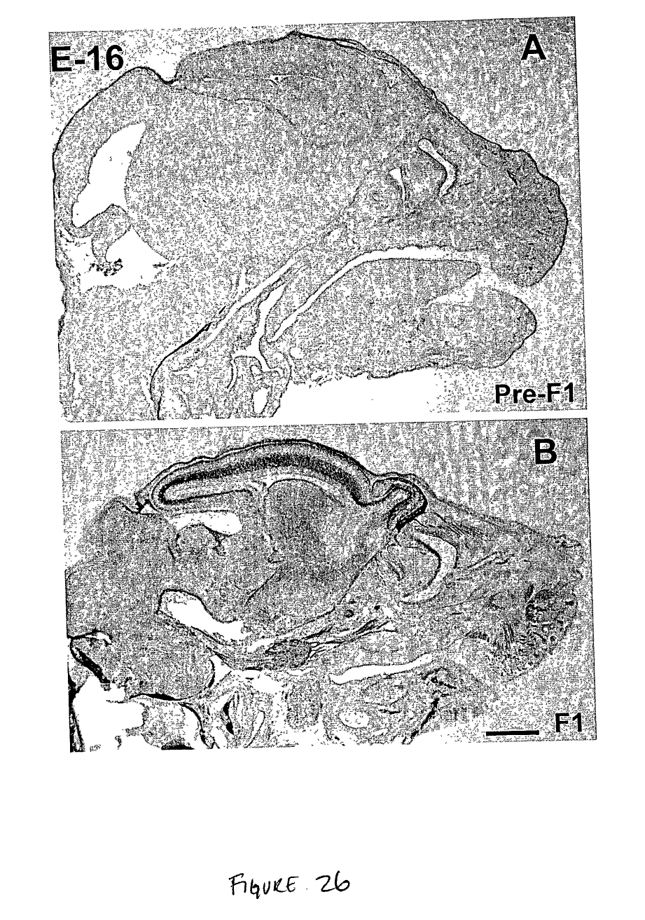

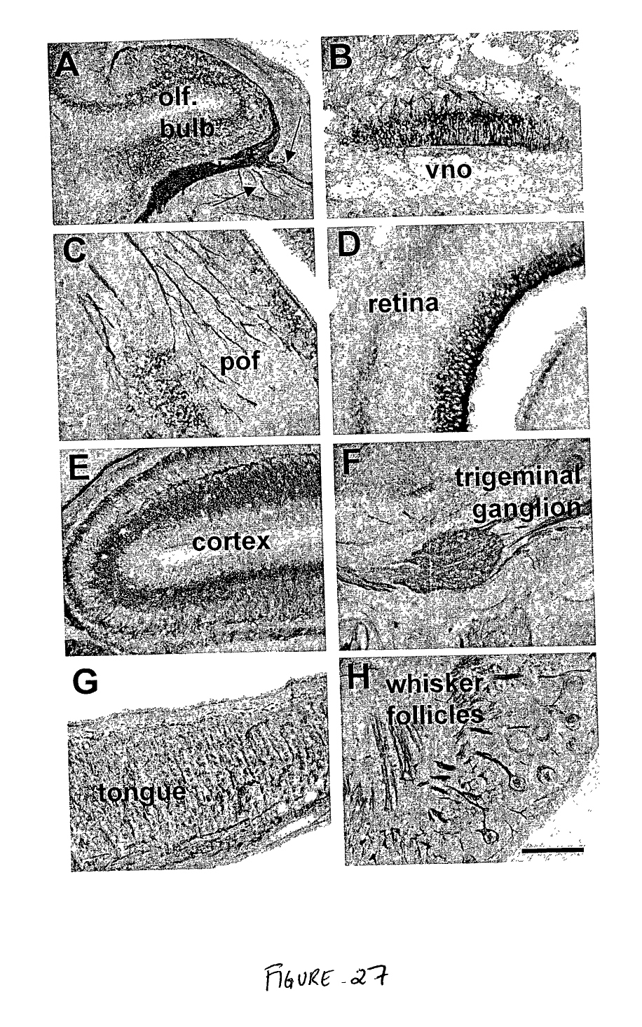

Using triton X-100 solubilized cell lysates, it was demonstrated that more than 50% of the AFAP-110 is in the insoluble fraction (FIG. 24) indicating that the majority of cellular AFAP-110 would be tightly associated with actin filaments. (Baisden et al., 2001, Oncogene, 20:6435-47). Thus, some free or soluble AFAP-110 may be available for recruitment to actin filaments. Neither expression of Src^{527F} nor cSrc exert a large change in the distribution of AFAP-110 among cytoskeletal and cytoplasmic cellular fractions. Interestingly, immunoprecipitation of AFAP-110 from the triton X-100 soluble and insoluble fractions followed by anti-phosphotyrosine blotting demonstrate that all the tyrosine phosphorylated AFAP-110 exists within the insoluble fraction, indicating that AFAP-110 may be a substrate for Src 527F when it is tightly associated with actin filaments (Flynn, unpublished data). This phosphorylation state of AFAP-110 is also in complex with Src via SH2 interactions. These data indicate that when AFAP-110 is recruited to actin filaments, it may also foster recruitment of soluble AFAP-110 proteins to actin filaments in a cooperative fashion. This in turn could affect actin filament cross-linking and facilitate the creation of larger signaling complexes.

### Example 17

### AFAP-110 Expression in Muscle Tissue

Western blot analysis of tissue culture cell lines demonstrated that AFAP-110 could be detected in almost every cell line examined, including fibroblast, epithelial and endothelial cell tines as well as cells of hematopoietic lineage (Flynn et al. 1995, J. Biol. Chem., 270:3894-3899). Expression patterns in cells are mainly limited to stress filaments and the cell membrane, although some peri-nuclear localization has been observed (Flynn et al. 1993, Mol. Cell Biol., 13:7892-7900). In tissues, AFAP-110 was expressed in a variety of tissues tested such as kidney, liver, or lung, but was detected at highest levels in brain and muscle tissue, including heart, skeletal muscle and intestine (smooth muscle). AFAP-110 was also detected in myoepithelium cells in breast tissue as shown in FIG. 25, indicating a strong representation in the basic types of muscle tissues.

### Example 18

### AFAP is Widely Expressed in Brain and Sensory Structures of Day 16 Mouse Embryos

To determine whether AFAP exhibited differential expression patterns during development, embryonic mouse heads were immunoreacted to reveal AFAP antigen (FIGS. 26-27).

Immunohistochemistry—Two adult mice were deeply anaesthetized with a 4% solution of chloral hydrate in normal saline (10 ml/kg) injected intraperitoneally. The mice were then perfused through the ascending aorta with a vascular rinse (0.9% NaCl, at room temperature), followed by 45 cc of a fixative consisting of 4% commercial formaldehyde (Fisher) and 0.5% zinc-dichromate (K & K Rare and Fine Chemicals, ICN), in 0.75% NaCl (pH 4.8, room temperature) as described previously (Berrebi and Spirou, 1998, Neuroscience, 83:535-554). The flow rate of the fixative was held at 5.1 cc/minute. The brains were left undisturbed for 1 hour prior to dissection from the cranium. Whole brains were cryroprotected overnight in 30% sucrose in saline (4° C.). Frozen parasagittal sections, 30 μm thick, were collected in series order on a sliding microtome and rinsed in 0.5 M Tris-HCl buffer (Tris, Sigma), pH 7.6. The free-floating sections were immunoreacted in Netwell dishes (Costar). Slices were first incubated in 5% normal donkey serum (NDS) with 0.5% triton-X-100 in 0.5M Tris for one hour at room temperature. Subsequently, the tissue was immersed for 48 hours, with continuous gentle agitation, in a rabbit anti-AFAP antibody (Ab F1) (6 μg/ml) mixed with 1% NDS and 0.1% triton X-100 in 0.5M Tris. These sections were then sequentially incubated for one hour in goat anti-rabbit IgG serum (1 :100) and then rabbit-PAP (1:100; Sternberger Monoclonals). The bound antigen was revealed by immersing the sections in 0.05% diaminobenzidine and 0.01% H₂O₂, pH 7.6, for 3-5 minutes. Between each step of the immunoreaction, sections were rinsed thoroughly in 0.5M Tris. Control sections were incubated in rabbit preimmune serum (PreF1) (6 μg/ml) and processed in parallel with the experimental sections. Specificity of the polyclonal Ab F1 to AFAP was determined by performing preabsorption controls using recombinant AFAP-110, generated in bacteria using the pGEX-6P-1 vector, as previously described (Qian et al. 2002, supra). The antisera was diluted 1:200 (5 μg/ml), preabsorbed overnight with 5 μg/ml of recombinant AFAP-110 in a final volume of 4 ml. Following preabsorption, tissue sections were incubated as described above. It was determined that 1.25 μg/ml of recombinant AFAP-110 was noticeably less efficient in blocking Ab F1 immunoreactivity (data not shown).

The immunoreacted tissue sections were mounted on glass slides, coverslipped and examined on an Olympus AX-70 microscope. Photographic images were obtained using an Optronics Magnafire digital camera.

Several mouse embryos (E-16) and mouse pups (P-3) were also analyzed by immunohistochemistry. The small size of these animals precluded perfusion through the ascending aorta, therefore heads were quickly removed and immersed in fixative solution for 24-48 hours. In order to reduce staining of endogenous peroxidase caused by residual blood in the tissue, sections from embryos and pups were immersed in a solution of 10% methanol and 3% H₂O₂ (Sigma) in 0.5M Tris for 5-10 minutes prior to the immunoreaction procedures described above.

Incubation in pre-immune serum revealed no specific immunostaining (FIG. 26A). FIG. 26 demonstrates immunohistochemical localization of AFAP in the E-16 mouse embryo head. Sections immunoreacted with the F1 antibody, however, revealed specific and intense AFAP-110 immunoreactivity (-IR) in widespread brain regions and many sensory structures (FIG. 26B) (Parasagittal sections. Scale bar=800 μm).

High magnification analysis of serial sections indicated very intense immunolabeling in neurons and processes of the main olfactory bulb and vomeronasal organ (vno), including the primary olfactory fibers that coursed from the peripheral sensory receptor epithelium toward the brain (FIGS. 27A-C). FIG. 27 depicts high magnification analysis of AFAP immunolocalization in E-16 mouse embryo head. AFAP expression is concentrated in several discrete regions of the embryonic brain and sensory structures. (A-C) Intense and specific AFAP-IR is observed in the olfactory bulb and vomeronasal organ (vno), as well as in the primary olfactory fibers (arrows) that originate from the sensory receptors in the nasal epithelium and line the ventral and rostral surface of the olfactory bulb (asterisks). AFAP is also immunolocalized to the (D) outer neural layers of the retina, (E) the neuropil of the middle layers of the neocortex, (F) the trigeminal ganglia, (G) the tongue and (H) the whisker follicles of the nose. Scale bar shown in panel H. Parasagittal sections, scale bars are (A, H) 250 μm; (B, E, G) 125 μm; (C, D) 100 μm and (F) 400 μm.

Dense AFAP-IR was also observed in the retina (FIG. 27D), neocortex (FIG. 27E), and hippocampus (not shown). In addition, AFAP was localized to trigeminal ganglia (FIG. 27F), the tongue (FIG. 27G) and whisker follicles in the nose (FIG. 27H).

### Example 19

### Expression of AFAP is Reduced in the Brains of P-3 Mouse Pups

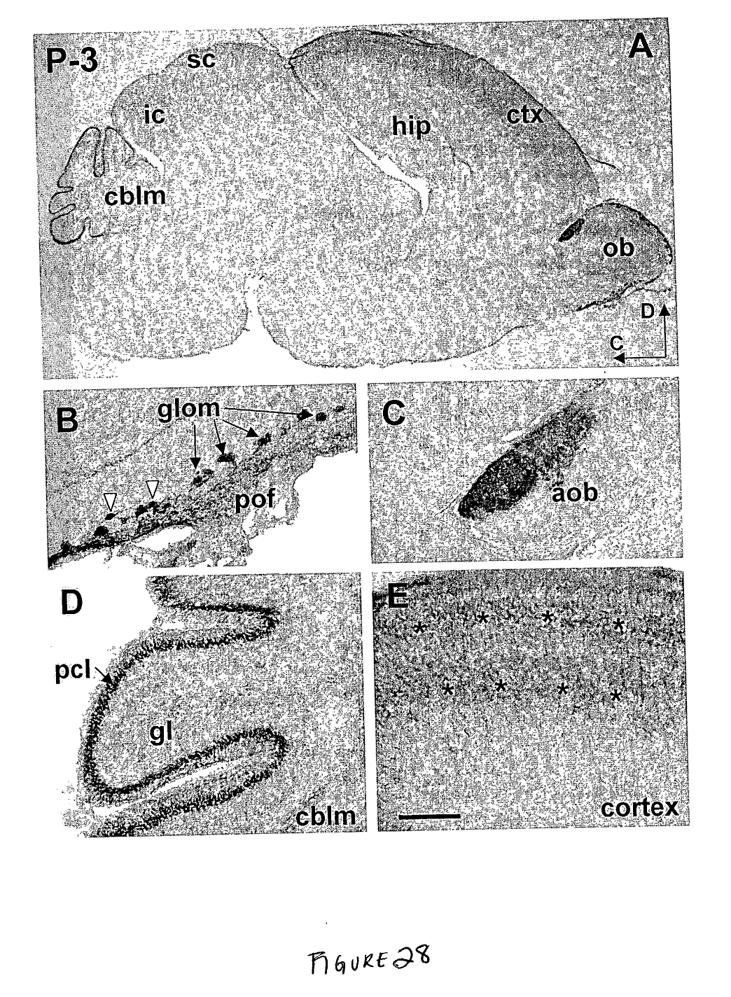

Immunoreacted sections of P-3 mouse brains indicated that AFAP expression was curtailed in its distribution relative to embryonic brain (FIG. 28). FIG. 28 shows immunohistochemical localization of AFAP in P-3 mouse brain. In FIG. 28A, at low magnification, specific AFAP-110-IR is detectable in the neocortex (ctx), cerebellum (cblm) and olfactory bulb (ob), where the accessory olfactory bulb is particularly well labeled. FIG. 28B shows that within the main olfactory bulb, AFAP localizes to primary olfactory fibers (pof) and well-defined olfactory glomeruli (glom; arrows). Occasional cell bodies, presumably neuronal in nature, interspersed among the glomeruli are AFAP-IR (arrowheads). FIG. 28C shows that the most intense AFAP-110-IR was localized to the glomeruli of the accessory olfactory bulb (aob). FIG. 28D demonstrates that at this age intense AFAP-IR is also present in the Purkinje cell layer (pcl) of the cerebellar cortex. FIG. 28E shows that although less prominent, specific AFAP-IR was also detected within the neuropil of the superficial and middle layers of the neocortex. (Parasagittal sections. C, caudal; cblm, cerebellum; D, dorsal; gl, granular layer; hip, hippocampus; ic, inferior colliculus; sc, superior colliculus. Scale bar shown in panel E. Parasagittal sections, scale bars are (A) 950 μm; (B) 150 μm; (C) 178 μm and (D, E) 125 μm). AFAP-IR was highest in the olfactory bulb and cerebellum, with weaker and somewhat patchy -IR observed in the neocortex (FIG. 28A). Within the olfactory bulb, AFAP was distinctly localized to main olfactory glomeruli and scattered cell bodies in the main olfactory bulb (FIG. 28B) and glomerular structures in the accessory olfactory bulb (FIG. 28C). Purkinje cells of the cerebellar cortex remain distinctly AFAP-IR at this age (FIG. 28D), as did the neuropil of the deeper layers of the neocortex (FIG. 28E).

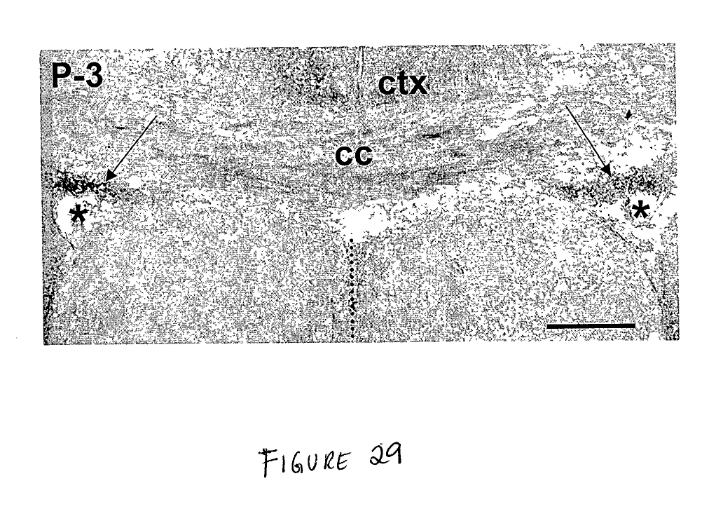

Interestingly, at P-3 we also observed specific AFAP-IR localized to the dorsal margins of the rostral aspect of the lateral ventricles, which was best viewed in the coronal plane of section (FIG. 29). This region is presumed to represent a proliferative zone that gives rise to neuronal precursor cells in mammals (Lois and Alvarez-Buylla, 1993, Proc. Natl. Acad. Sci USA, 90:2074-2077).

FIG. 29 demonstrates that in P-3 mouse pups, AFAP was localized (arrow) to the margins of the rostral aspect of the lateral ventricles (asterisks), a proliferative region for neuronal cells in early development (Coronal section; dotted line indicates midline. Cc, corpus callosum; ctx, cortex (lobe), Scale bar =200 μm).

### Example 20

### AFAP Expression is Restricted to the Olfactory Bulb in Adult Mouse Brain

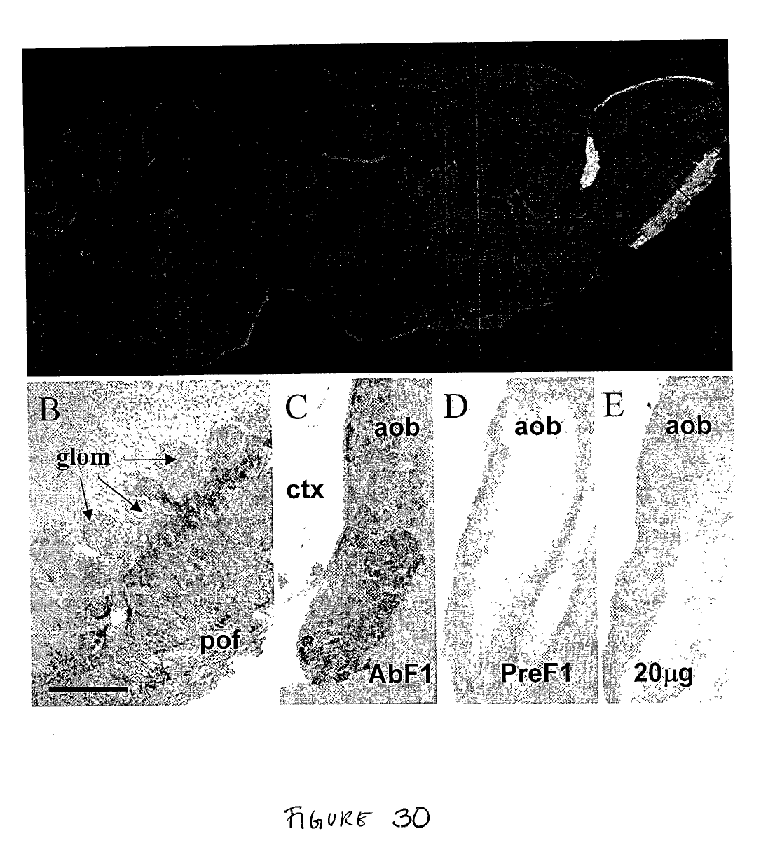

In adult mice, only the main and accessory olfactory bulbs displayed intense AFAP-IR, with only weak expression remaining in the hippocampus and cerebellar cortex that was barely detectable above background signal levels (FIG. 30). FIG. 30 demonstrates immunohistochemical localization of AFAP in adult mouse brain. FIG. 30A demonstrates that in adult mice, intense AFAP-IR is confined to the main and accessory olfactory bulbs. Boxed areas enlarged in B and C. FIG. 30B demonstrates that at higher magnification it is evident that only primary olfactory fibers (pof) and glomeruli (glom., arrows) of the main olfactory bulb display intense AFAP immunostaining. Olfactory neurons within the bulb are immunonegative. FIG. 30C shows that AFAP expression remains particularly intense in glomerular structures within the accessory olfactory bulb (aob) in adults. FIG. 30D shows the results of immunohistolabeling using 5 μg/ml preimmune F1 antibody demonstrate essentially background levels of immunoreactivity in the accessory olfactory bulb. FIG. 30E demonstrates the results with preincubation of Ab F1 (5 μg/ml) with 5 μg/ml of recombinant AFAP-110 generated from bacteria significantly reduced immunoreactivity associated with Ab F1. Parasagittal sections are shown. ctx, cortex. Scale bar shown in panel B. Scale bars are (A) 1600 μm; (B) 200 μm; (C) 250 μm; (D) 212 μm and (E) 212 μm.

In the olfactory bulb, the AFAP was associated with primary olfactory fibers that arose from the olfactory receptor neurons of the peripheral sensory epithelium and lined the ventral and rostral surface of the bulb (FIG. 30B), and within glomerular structures throughout the main and accessory olfactory bulbs (FIGS. 30B-C). It is noteworthy that neuronal cell bodies were immunonegative throughout the olfactory bulb of adult mice. As a control, Preimmune F1 antibodies were used or Ab F1 was preincubated with recombinant AFAP-110 produced in bacteria and purified as previously described (Qian et al., 2002, supra). Both controls resulted in reduction of immunostaining to approximately background levels. Neither Preimmune F1 antibodies (FIG. 30D), nor preincubation of recombinant AFAP-110 with Ab F1 revealed significant immunoreactivity in either adult brain or in the accessory olfactory bulb (FIG. 30E).

### Example 21

### AFAP-120 is an Alternatively Spliced Isoform of AFAP-110

The human form of AFAP-110 exists on chromosome 4p16.1 (Han et al., submitted; NCBI annotation project, www.ncbi.nlm.nih.gov; International human genome sequencing consortium, 2001). A Blast search was done using cDNA sequence representative of the human AFAP-110 against genomic sequences and a match was identified in the Chromosome 4 working draft.

Database analysis—The cDNA sequence of chicken AFAP-110 (Genbank accession #L20303) and chicken AFAP-120 (Genbank accession #L20302) are highly homologous to the reported cDNA sequence of human AFAP-110 (Genbank accession #NM_{—}021638; Han et al., submitted). The cDNA sequence of human AFAP-110 was used to search for homologous sequences deposited by the human genome project using Blast search. Homologous sequence was identified in the working draft of Homo sapiens chromosome 4 (Accession: NT_{—}006362) deposited in the NCBI database (www.ncbi.nlm.nih.gov). The genomic sequence deposited represented the reverse, complement strand sequence that contained sequence homologous to the human AFAP-110 cDNA. Alignments of coding and noncoding sequence were uncovered and shown in the report of the search against chromosome 4, permitting identification of intronic sequences that separate the coding sequences for Ser⁵¹⁰ and Phe⁵¹¹. Sequence homologies were aligned using DNASIS and predicted amino acid coding sequences determined using the PROSIS program (Hitachi, Inc.).

Coding sequence representative of amino acids 301-730 were identified in the genomic sequence, while upstream sequences encoding the amino terminal region of hAFAP-110 were unavailable due to the presence of gaps and undeposited sequence (data not shown). The available 3′ sequence indicated the coding sequences with intron and exon boundaries that correspond to individual functional domains within hAFAP-110 (Table 1), which were consistent with the predicted coding sequence of the human homologue of AFAP-110 (hAFAP-110) identified by Han et al. (Han et al., submitted), as well as the avian homologue of AFAP-110 identified by Flynn et al. (Flynn et al., 1993, supra GenBank #L20303) and the corresponding functional domain structure predicted by Baisden et al. (Baisden et al., 2001 a, supra).

TABLE 1

| Amino acid coding sequence/exon | Corresponding functional domain |
|---|---|
| a.a. 302 → 351 | Carboxy terminal half of the STK |
| | (ser/thr kinase target) domain |
| a.a. 352 → 422 | Majority of the carboxy terminal |
| | Pleckstrin homology domain |
| a.a. 423 → 510 | End of carboxy terminal pleckstrin |
| | homology domain (including highly |
| | conserved Trp residue) and the car- |
| | boxy terminal SH2 binding motif |
| | (Y⁴⁵¹/Y⁴⁵³) |
| a.a. 511 → 548 | Coil/turn region |
| a.a. 549 → 603 | All of the leucine zipper motif |
| | (including 11 amino terminal and 5 |
| | carboxy terminal amino acids) |
| a.a. 604 → 667 | Actin binding domain |
| a.a. 668 → 722 | Additional carboxy terminal seq- |
| | uence, unknown function |
| a.a. 723 → end (730) | Termination sequence and 3′ |
| | noncoding sequence |

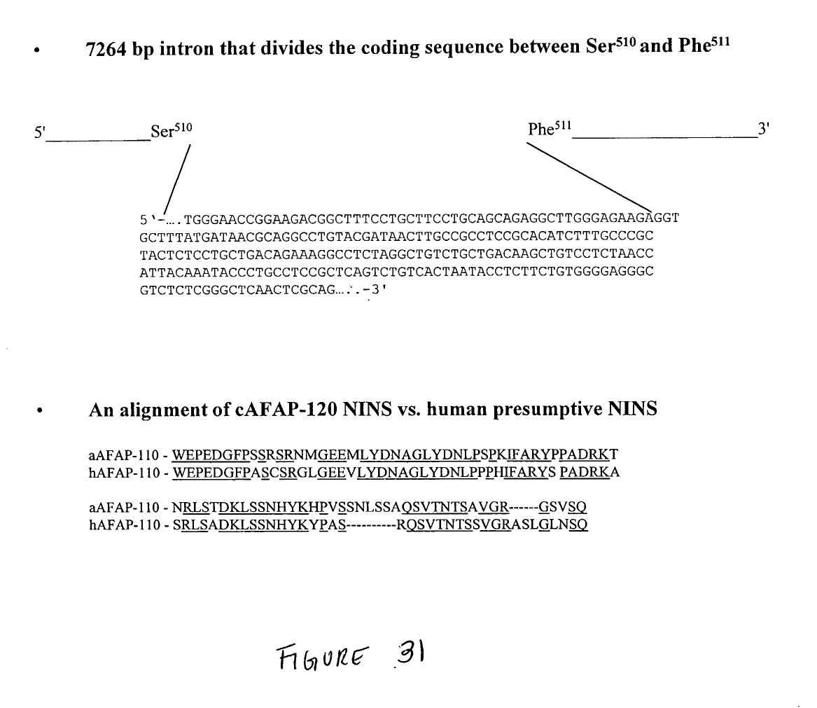

FIG. 31 demonstrates that AFAP-120 represents a splice variant of AFAP-110. The intron between Ser⁵¹⁰ and Phe⁵¹¹ contains coding sequence for the human NINS. FIG. 31A is a schematic demonstrating coding sequence contained within an intron that divides Ser⁵¹⁰ and Phe⁵¹¹ in the human AFAP gene. FIG. 31B is the predicted human cDNA sequence for the NINS encodes polypeptide sequences that are homologous to the known avian AFAP-120 NINS sequence. It was noted that a 7264 bp intronic sequence separated coding sequences that define the codons for Ser⁵¹⁰ and Phe⁵¹¹ (FIG. 31A). Previous data from Flynn et al., demonstrated that the NINS coding sequence identified in the avian AFAP-120 homologue was placed between Ser⁵¹⁰ and Phe⁵¹¹ (Flynn et al., 1995, supra). Therefore, the 7264 bp intronic sequence was analyzed for the presence of homologous NINS coding sequence, by performing a computer based search of this intron sequence against avian sequence which encodes the NINS. The analysis indicated a short genomic sequence that was predicted to encode a protein which was 76% identical and 90% homologous to the avian AFAP-120 NINS sequence over 86 amino acids (FIG. 31). An analysis of this sequence reveals that it is predicted to be two amino acids shorter than the avian NINS, encoding 84 amino acids. The homologous proline-rich region previously hypothesized to play an important role in protein-protein interactions was retained. The 7264 bp intron that contains the human NINS predicted sequence is flanked by a 5′ GT and 3′ AG sequence which are consistent with elements known to be associated with introns (data not shown).

### Example 22

### Generation of an Antibody Against AFAP-120

Protein Characterization and Analysis—The avian cDNA sequence encoding the NINS was subcloned in the pGEX-6P-1 vector (Pharmacia, Inc.) and expressed as a GST-encoded fusion protein in DH5α bacteria. The avian NINS coding sequence was amplified by PCR and subcloned into the pGEX-6P-1 vector and a purified avian NINS protein generated after digestion with Precission cut enzyme and dialysis against PBS. The purified protein was quantitated and used as an antigen for challenge into rabbits. Rabbit serum was purified and the resulting antibodies (α-NINS) used for western blot analysis and contrasted against Ab F1, which is immunoreactive with coding sequences common to both AFAP-110 and AFAP-120 (Flynn et al., 1995, supra; Qian et al., 1999, supra). The NINS was dialyzed against phosphate buffered saline, quantitated by Coomasie staining and used as an antigen for challenge in rabbits (Spring Valley Labs, Inc.). The resulting antibody was purified as previously described (Flynn et al., 1993, supra). Ab F1 and PreF1 were prepared as previously described (Flynn et al., 1993, supra). Whole mouse brains were lysed with RIPA buffer, as previously described (Flynn et al., 1995, supra). A rat brain was grossly dissected into 3 compartments, the olfactory bulb, cerebellum and hindbrain. Brain tissue was lysed using modified RIPA as described previously (Flynn et al., 1992, supra). Tissue lysates were resolved using 8% SDS PAGE. Western transfer and Western blot analysis with Ab F1 and PreF1 was done as described previously (Flynn et al., 1992, supra). Bound primary antibody was quantitated using a horseradish peroxidase conjugated secondary antibody followed by detection using chemiluminescence.

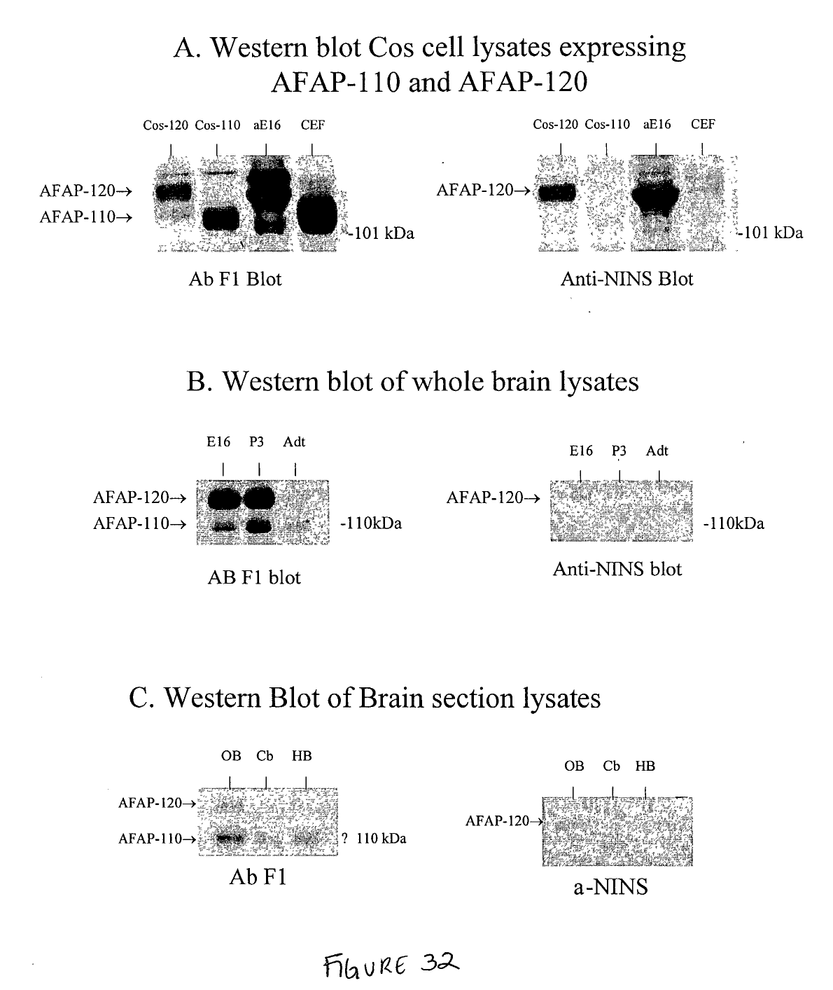

Ab F1 immunoreacts with an epitope common to both AFAP-110 and AFAP-120 (Flynn et al., 1995, supra; Qian et al., 1999, supra). AFAP-110 and AFAP-120 were expressed in Cos-7 cells using the pCMV-1 vector and CMV promoter, as previously described (Guappone et al., 1996, supra). In addition, CEF and day 16 embryonic chicken brain lysates were isolated as previously described (Flynn et al., 1995, supra). FIG. 32 shows the results of Western blot analysis for AFAP-110 and AFAP-120 in brain. Western blot analysis with α-NINS reveals AFAP-120 expression correlates with the concentrated AFAP expression detected by Ab F1 immunohistochemistry. (A) the α-NINS antibody recognizes AFAP-120. 50 μg of Cos-7 cell lysates expressing the avian isoform of AFAP-110 and AFAP-120, or chick embryo fibroblast cells and chick embryo (E-16) brain lysates were resolved by 8% SDS-PAGE and western blot analysis performed with Ab F1 or α-NINS. Cos-120—Cos cells expressing AFAP-120; Cos-110—Cos cells expressing AFAP-110; CEF—chick embryo fibroblasts; aE-16—Day 16 avian chick embryonic brain. (B) Whole brain lysates were generated and 50 μg of the lysates were resolved by 8% SDS-PAGE for western blot analysis with Ab F1 or α-NINS. E-16—day 16 mouse embryo brain, P-3—day 3 mouse pup brain, Adt—3 month old adult mouse brain. (C) The olfactory bulb, cerebellum or hindbrain surgically isolated from an adult rat brain, lysed and 50 μg resolved by 8% SDS-PAGE for western blot analysis using Ab F1 or α-NINS. OB—olfactory bulb, Cb—cerebellum, HB—hindbrain. The lysates were resolved by SDS-PAGE and probed with Ab F1 and anti-NINS (FIG. 32A). The data demonstrate that Ab F1 is immunoreactive with the 110 kDa AFAP-110 protein in CEF, as well as in Cos-7 cell lysates that express AFAP-110. Ab F1 is also immunoreactive with a 120 kDa protein found in E-16 chicken brain and with AFAP-120 expressed in Cos-7 cells (FIG. 32A). However, the anti-NINS antibody was only immunoreactive with the 120 kDa protein found in E-16 brain and Cos-7 cells expressing AFAP-120. These data indicate that α-NINS can distinguish avian AFAP-120 from AFAP-110.

Whole brains were extracted from an E-16 embryo, a day 3 pup and an adult mouse, homogenized, fifty μg of tissue lysates resolved by 8% SDS-PAGE and processed for western blot analysis with Ab F1 and α-NINS. Ab F1 immunoreactivity indicated that AFAP expression levels (both AFAP-110 and AFAP-120) were relatively high in the embryo and pup brain, compared to adult mouse brain (FIG. 35B). Western blot analysis with α-NINS demonstrated that AFAP-120 is expressed at higher steady state levels in embryo and pup brain compared to adult brain. α-NINS Immunoreactivity with murine AFAP-120 was consistently lower than seen with avian AFAP-120, indicating that there may be some minor sequence differences in the epitope that affects avidity between species. To determine whether the 120 kDa isoform might be associated with upregulation of protein expression, an adult rat brain was removed from the skull and the olfactory bulb, cerebellum and hindbrain were grossly dissected. Previous data indicate that adult rat brain exhibits expression patterns of AFAP that are identical to mouse brains (Baisden et al., 2001 a, supra). Western blot analysis with Ab F1 demonstrates that the 110 kDa AFAP-110 isoform can be detected in each of the gross tissue lysates; however, a 120 kDa isoform was prominent in lysates of the olfactory bulb (FIG. 35C). Western blot analysis with α-NINS confirm that AFAP-120 could be uniquely detected in the olfactory bulb and not in the cerebellum or hindbrain. A smaller protein of about 110 kDa can be detected with α-NINS in the cerebellum sample. This may have resulted from some AFAP-120 expression and subsequent protein degradation, as has been described for AFAP-120 previously (Flynn et al., 1995, supra), or it may represent a cross-immunoreactive and distinct form of AFAP-120. Collectively, these data confirm that AFAP expression levels are developmentally regulated in the brain and indicate that concentrated expression levels may correlate with AFAP-120 expression.

1. An isolated nucleic acid molecule comprising
a) a nucleic acid sequence according to FIGS. 1A-C or FIG. 3C;
b) a nucleic acid sequence according to FIGS. 1A-C or FIG. 3C, wherein the base T is substituted by the base U;
c) a nucleic acid encoding an amino acid sequence according to FIG. 2, FIG. 3A or FIG. 3B;
d) a nucleic acid sequence complementary to any of sequences (a)-(c);
e) a nucleic acid sequence which specifically hybridizes to any of sequences (a)-(d) under stringent conditions;
f) a nucleic acid sequence fragment of the sequence of (c) wherein said fragment comprises at least one AFAP domain; or
g) a nucleic acid sequence greater than about 87% identical to a nucleic acid encoding chicken AFAP-110 nucleic acid.

2. A nucleic acid according to claim 1(e) wherein said nucleic acid sequence localizes to chromosome 4q16.1

3. A nucleic acid according to claim 1(g) wherein said sequence encodes a human or mouse AFAP polypeptide.

4. A nucleic acid splice variant of the sequence according to claim 1(a) or 1(b) wherein said sequence comprises a sequence encoding a human NINS amino acid sequence.

5. A nucleic acid sequence according to claim 4 or a fragment thereof, wherein said sequence or fragment thereof hybridizes to an AFAP-120 transcript and not to an AFAP-110 transcript.

6. The nucleic acid according to claim 1(c) or 1(f) wherein said amino acid sequence comprises one or more insertions, deletions, and substitutions and wherein said nucleic acid specifically hybridizes to a human AFAP transcript under stringent conditions.

7. The nucleic acid according to claim 1(c) or 1(f) wherein said nucleic acid is fused in frame to a nucleic acid encoding a heterologous polypeptide.

8. The nucleic acid according to claim 1(f) wherein said one or more AFAP domains is selected from the group consisting of: a human AFAP-110 WW binding domain; a human AFAP-110 SH2 binding domain; a human AFAP-110 SH3 binding domain; a human AFAP-110 PH1 and/or PH2 domain; a human AFAP-110 leucine zipper; a human AFAP-110 actin-binding domain; a human AFAP serine/threonine kinase target domain and a human AFAP nuclear export domain

9. A nucleic acid according to claim 1, wherein said nucleic acid is stably associated with a solid support.

10. The nucleic acid according to claim 6 wherein said heterologous polypeptide is Green Fluorescent Protein.

11. A vector comprising a nucleic acid according to any of claims 1-2.

12. The vector according to claim 11, wherein said vector is an expression vector.

13. A host cell comprising a nucleic acid according to claim 11.

14. A tissue comprising the host cell according to claim 13.

15. A non-human animal comprising a host cell according to claim 13.

16. A polypeptide encoded by a nucleic acid according to claim 12.

17. A method for producing a polypeptide, comprising introducing an expression vector according to claim 12 into a host cell and culturing said host cell under conditions which allow for the expression of said polypeptide.

18. The polypeptide according to claim 16, wherein said polypeptide is phosphorylated.

19. The polypeptide according to claim 16, wherein said polypeptide is stably associated with a solid support.

20. An antibody which specifically recognizes a polypeptide according to claim 16.

21. An antibody which specifically recognizes a polypeptide according to claim 18.

22. The antibody according to claim 21, wherein said antibody does not recognize a non-phosphorylated form of said polypeptide.

23. The antibody according to claim 21, wherein said antibody modulates one or more AFAP activities upon binding to an AFAP polypeptide.

24. The antibody according to claim 21, wherein said antibody specifically recognizes AFAP-120 and not AFAP-110.

25. The antibody according to claim 21, wherein said antibody recognizes an epitope within amino acids 1-55 of the human AFAP-110 polypeptide.

26. The antibody according to claim 21, wherein said antibody recognizes an epitope within a human AFAP SH3 binding motif; a WW binding domain; an AFAP-110 SH2 binding domain; an AFAP-110 PH1 and/or PH2 domain, an H-SH2 domain, an AFAP-110 leucine zipper, or a AFAP-110 actin-binding domain or modified or variant form thereof.

27. A hybridoma cell secreting an antibody according to claim 21.

28. A method of screening for enhanced risk for or presence of a pathology associated with an aberrant AFAP signaling pathway in a test patient, comprising:
a) obtaining a biological sample from said patient;
b) contacting said sample with a molecular probe reactive with an AFAP biomolecule and detecting the reactivity of the molecular probe.

29. The method according to claim 28, wherein said pathology is cancer or obesity.

30. The method according to claim 29, wherein said cancer is selected from the group consisting of: breast cancer, colon cancer, prostate cancer, lung cancer, a cancer involving neural cells, Ewing sarcoma and rhabdomyosarcoma.

31. The method according to claim 28, wherein said AFAP biomolecule is a nucleic acid encoding an AFAP polypeptide or is an AFAP polypeptide, modified form thereof, or variant form thereof.

32. The method according to claim 28, wherein said molecular probe is a nucleic acid.

33. The method according to claim 32, wherein said molecular probe is an antibody.

34. The method according to claim 28, wherein an increase or a decrease in the amount of an AFAP biomolecule in said sample from said test patient compared to a normal patient provides an indication of an increased risk for the presence of said pathology.

35. The method according to claim 28, wherein said molecular probe reacts with a modified form of an AFAP polypeptide but does not react with the unmodified form of the AFAP polypeptide and wherein detection of said modified form indicates an increased risk for the presence of said pathology.

36. The method according to claim 28, wherein said molecular probe is used to detect a mutant form of an AFAP gene.

37. The method according to claim 36, wherein said mutant form is an AFAP transcript.

38. A method for identifying an agent which is capable of binding to an AFAP polypeptide, modified form thereof, or variant thereof, comprising:
a) reacting a AFAP polypeptide, a fragment thereof comprising one or more AFAP domains, a modified form thereof, or a variant form thereof, with at least one agent; and
b) detecting changes in one or more AFAP activities when compared to said one or more activities of said AFAP polypeptide, fragment thereof comprising one or more AFAP domains, modified form thereof, or variant thereof, which has not been contacted with the agent.

39. The method according to claim 38, wherein said agent is an antibody or antigen-binding fragment thereof.

40. The method according to claim 39,
wherein said method is performed under conditions which permit the formation of complexes between said agent and the AFAP polypeptide, fragment thereof comprising one or more AFAP domains, modified form thereof, or variant thereof, and
wherein agents are selected which modulate the formation of such complexes, by measuring the amount of one or more of: unbound agent, complexes, and unbound AFAP polypeptide, fragment thereof comprising one or more AFAP domains, modified form thereof, or variant thereof.

41. A method for identifying an agonist or antagonist of the interaction between an AFAP polypeptide, a fragment thereof comprising one or more AFAP domains, a modified form thereof, or variant thereof, and a substance which binds to said polypeptide, fragment thereof comprising one or more AFAP domains, modified form thereof, or variant thereof, comprising:
a) providing a known concentration of said AFAP polypeptide, fragment thereof comprising one or more AFAP domains, modified form thereof, or variant thereof;
b) providing a substance which is capable of specifically binding to the AFAP polypeptide, fragment thereof comprising one or more AFAP domains, modified form thereof, or variant thereof, under conditions which permit the formation of complexes between the substance and the AFAP polypeptide, fragment thereof comprising one or more AFAP domains, modified form thereof, or variant thereof; and
c) assaying for one or more of: complexes, free substance, and non-complexed AFAP polypeptide, fragment thereof comprising one or more AFAP domains, modified form thereof or variant thereof or one or more activities of an AFAP polypeptide.

42. The method according to claim 41, wherein said substance is a polypeptide.

43. The method according to claim 42, wherein said substance is Src, actin, PKCα, or Rho.

44. The method according to claim 42, wherein said substance is an antibody.

45. The method according to claim 41, wherein an agonist or antagonist is identified which constitutively activates Src.

46. The method according to claim 41, wherein an agonist or antagonist is identified which modulate the interactions of an AFAP polypeptide, modified form thereof, or variant form thereof, with the SH3 domain of Src.

47. The method according to claim 41, wherein an agonist or antagonist is identified which affects binding of an AFAP polypeptide comprising one or more PH domains, modified form thereof, or variant thereof, to PKCα.

48. A pharmaceutical composition comprising an agonist or antagonist according to any of claims 38-40.

49. A pharmaceutical composition comprising an agonist or antagonist according to any of claims 41-47.
